(19) United States
(12) Patent Application Publication
(10) Pub. No.: US 2016/0176859 A1
(43) Pub. Date: Jun. 23, 2016

(54) High-Throughput Assay for Identifying Small Molecules that Modulate AMP-activated Protein Kinase (AMPK)

(71) Applicants: North Carolina Central University, Durham, NC (US); The University of North Carolina at Chapel Hill, Chapel Hill, NC (US)

(72) Inventors: Jonathan Z. Sexton, Raleigh, NC (US); Jay E. Brenman, Chapel Hill, NC (US)

(21) Appl. No.: 15/040,412

(22) Filed: Feb. 10, 2016

Related U.S. Application Data
(63) Continuation of application No. PCT/US2015/013490, filed on Jan. 29, 2015.
Application No. PCT/US2015/013490 is a continuation-in-part of application No. PCT/US2014/051022, filed on Aug. 14, 2014.
(60) Provisional application No. 61/865,729, filed on Aug. 14, 2013.

(51) Int. Cl. C07D 417/06; G01N 21/64; G01N 33/573

(52) U.S. Cl. CPC C07D 417/06; G01N 33/573; G01N 21/6428; G01N 2333/912; G01N 2201/12

(57) ABSTRACT

The present invention provides an in vitro method for identifying a compound that modulates adenosine monophosphate-activated protein kinase (AMPK) for the manufacture of a diagnostic or therapeutic agent. The present invention further provides an assay for identifying a compound that modulates AMPK.

### STATEMENT OF PRIORITY

This application claims priority to International Application No. PCT/US2014/51022, filed Aug. 14, 2014, the entire content of which is incorporated by reference herein.

### STATEMENT OF GOVERNMENT SUPPORT

This invention was made with government support under grant number NS080108 from the National Institutes of Health. The government has certain rights to this invention.

### FIELD OF THE INVENTION

The present invention relates to methods and assays for identifying compounds, in particular, small molecules, that bind to and/or modulate adenosine monophosphate-activated protein kinase (AMPK). The present invention also relates to compounds that modulate AMPK and can be used to treat various disorders.

### BACKGROUND OF THE INVENTION

5′ adenosine monophosphate-activated protein kinase or AMP-activated protein kinase (AMPK) is a heterotrimeric serine-threonine kinase that regulates anabolic and catabolic pathways in eukaryotes (Carling D, Thornton C, Woods A, Sanders M J. AMP-activated protein kinase: new regulation, new roles? Biochem J 2012; 445(1):11-27; Oakhill J S, Scott J W, Kemp B E. AMPK functions as an adenylate charge-regulated protein kinase. Trends Endocrinol Metab 2012; 23(3):125-3). The earliest AMPK modulators, AICAR and Compound C, were not specifically developed for AMPK and have off-target effects (Kim M, Tian R. Targeting AMPK for cardiac protection: opportunities and challenges. J Mol Cell Cardiol 2011; 51(4):548-53; Zhou G, Myers R, Li Y, et al. Role of AMP-activated protein kinase in mechanism of metformin action. J Clin Invest 2001; 108(8):1167-74). Compound C, which was identified in a library screen for AMPK inhibitors, binds the canonical ATP-binding catalytic site in numerous kinases (Kim M, Tian R. Targeting AMPK for cardiac protection: opportunities and challenges. J Mol Cell Cardiol 2011; 51(4):548-53; Zhou G, Myers R, Li Y, et al. Role of AMP-activated protein kinase in mechanism of metformin action. J Clin Invest 2001; 108(8):1167-74; Handa N, Takagi T, Saijo S, et al. Structural basis for compound C inhibition of the human AMP-activated protein kinase alpha2 subunit kinase domain. Acta Crystallogr D Biol Crystallogr 2011; 67(Pt 5):480-7). Strategies designed to identify AMPK modulators that target AMPK-unique protein domains, therefore, have the potential to identify molecules that are more selective than those that bind the canonical ATP-binding catalytic site found throughout the kinome.

### SUMMARY OF THE INVENTION

Embodiments of present invention are directed to methods for identifying a compound that modulates adenosine monophosphate-activated protein kinase (AMPK) for the manufacture of a diagnostic or therapeutic agent. The methods may include: (a) contacting a sample comprising AMPK with a luminescent agent known to bind AMPK; (b) contacting the sample from (a) with a compound of interest; and (c) comparing the luminescence in the sample prior to contacting the sample with the compound of interest to the luminescence in the sample after contacting the sample with the compound of interest. A decrease in luminescence measured after contacting the sample with the compound of interest indicates that the compound of interest is a modulator of AMPK.

Embodiments of the present invention also provide assays for identifying a compound that modulates AMPK for the manufacture of a diagnostic or therapeutic agent which include screening a compound of interest for its effect to displace a fluorescent AMPK ligand bound to AMPK. Displacement of the fluorescent AMPK ligand results in a decrease in luminescence, which indicates that the compound of interest is a modulator of AMPK.

Embodiments of the present invention further provide methods for identifying a compound that modulates AMPK including (a) contacting a sample comprising AMPK with a luminescent agent known to bind AMPK, (b) contacting the sample from (a) with a compound of interest, and (c) comparing the luminescence in the sample prior to contacting the sample with the compound of interest to the luminescence in the sample after contacting the sample with the compound of interest, wherein (i) a decrease in luminescence after contacting the sample with the compound of interest indicates that the compound of interest is a modulator of AMPK, or (ii) an increase in luminescence after contacting the sample with the compound of interest indicates that the compound of interest is a modulator of AMPK when the luminescent agent is environmentally sensitive.

Embodiments of the present invention also provide methods of modulating the activity of adenosine monophosphate-activated protein kinase (AMPK), comprising contacting a sample comprising AMPK with a compound selected from the group consisting of compounds as described herein.

Embodiments of the present invention further provide methods of treating diabetes, obesity, metabolic syndrome and cancer comprising administering a compound selected from the group consisting of compounds as described herein as well as pharmaceutical compositions including the identified compounds.

Embodiments of the present invention provide novel compounds and compounds that modulate AMPK and can be used to treat various disorders.

Embodiments of the present invention provide the compounds described herein formulated as pharmaceutical compositions including a pharmaceutically acceptable carrier.

Embodiments of the present invention also provide kits including the elements necessary to carry out the processes described above.

### BRIEF DESCRIPTION OF THE DRAWINGS

FIG. 1. AMPK-β and AMPK-γ subunits co-eluted with His-tagged AMPK-α. In agreement with published data, rat AMPK-β₁ and rat AMPK-γ₁ have similar apparent molecular weights Neumann D, Woods A, Carling D, Wallimann T, Schlattner U. Mammalian AMP-activated protein kinase: functional, heterotrimeric complexes by co-expression of subunits in Escherichia coli. Protein Expr Purif 2003; 30(2):230-7.

FIG. 2. ADP competes with MANT-ADP for binding to AMPK. (A) Simplified cartoon showing MANT-ADP fluorescence increasing upon binding to protein. AMPK-α and AMPK-β subunits and additional nucleotide-binding sites are omitted for clarity. (B) MANT-ADP fluorescence increased after addition of regulatory fragment or full-length AMPK (excitation=360 nm). Background fluorescence from protein was subtracted from raw data prior to plotting the corrected spectra shown above. A 460 nm emission filter was used for subsequent experiments. (C) ADP inhibited the increase in MANT-ADP fluorescence. (D) Full-length AMPK provided a slightly greater assay window. Z′-factors>0.6. (B-C) n=5 wells per data point. (D) n=6 wells per data point. Data points are mean±standard deviation. Some of the standard deviations are too small to be visible when plotted on this scale. RFUs, relative fluorescence units.

FIG. 3. (A) MANT-ADP fluorescence decreased as the ionic strength of the assay solution increased. In the absence of NaCl, 0.01% Triton had no effect on MANT-ADP fluorescence. (B) The assay window increased linearly as concentrations of AMPK, ADP, and MANT-ADP were increased at a constant molar ratio. (A) n=4 wells per data point; (B) n=6 wells per data point. Data points are mean±standard deviation. Z′-factors≧0.6.

FIG. 4. Full-length AMPK tolerated 0-2% DMSO. n=6 wells per data point. Data points are mean±standard deviation.

FIG. 5. (A) In a set of sixteen plates from the small molecule library, two of 5120 molecules inhibited MANT-ADP fluorescence by more than 50% (yellow arrows). Each plate included 32 positive and 32 negative controls (green and red circles, respectively). Negative inhibition most likely indicates small molecule autofluorescence. Many of the autofluorescent molecules were plotted on a logarithmic y-axis to conserve space. The average Z′-factor for the plates shown above is 0.58. (B) Histograms and box plots show distribution statistics for the positive control ADP (green), vehicle (red), and library molecules (black). Whiskers indicate the range; boxes indicate data between the first and third quartile.

FIG. 6. (A-C) Three of the five positive hits produced sigmoidal dose responses in the presence of full-length AMPK and the regulatory fragment. DMSO and ADP controls were included on each plate and used to define 0 and 100% inhibition, respectively. n=5 wells per data point. Data points are mean±standard deviation.

FIG. 7. Western blot of treated HEK cells with compound 1 (STL035166), showing decreased phospho-AMPKα, while compound 2 (STK740822) shows increased phospho-AMPKα. (standardized to tubulin).

FIG. 8. Quantitative increase is in phospho-AMPK upon treatment with Compound A (STK740822) and Compound B (STL035166) in human Hek293 cells.

FIG. 9. Triplicate similarity screen. (A). Analogs that reproducibly inhibited MANT-ADP's protein-bound signal are highlighted in red. These three analogs contain a common scaffold (inset structure) and were screened at a final concentration of 45 μM. Parallel dose responses for analogs of STK740822. Data shown are mean % inhibition of MANT-ADP's protein-bound signal±σ. n=3. (C) To demonstrate direct interference with the fluorescent probe, we plotted the mean % decrease of MANT-ADP's unbound signal in the presence of various analogs. The background signal of the assay buffer (0 μM MANT-ADP, 0 μM AMPK, 0 μM analog) was used to define 100% inhibition. n=2.

FIG. 10. MANT-ADP assay. (A) Isothermal titration calorimetry was used to confirm that ADP binds two sites on full-length AMPK. The calculated dissociation constants (13 μM at both sites) are similar to published dissociation constants of 10±2 μM (both sites) for MANT-AMP in the presence of AMPK in 25 mM Tris (pH 8) (22). n=1. (B) The same concentration of ADP can be used to displace MANT-ADP from both full-length AMPK and the regulatory fragment. (C) A greater concentration of BAS02250954 is needed to displace MANT-ADP from full-length AMPK. (B-C) Binding of ADP and BAS02250954 to the regulatory fragment and full-length AMPK was compared in parallel on one 384-well plate. n=3. Data points shown are mean±σ.

FIG. 11. Dose-dependent inhibition of purified p-AMPK. (A) Two analogs inhibit phosphorylation of p-GST-ACC peptide. (B) BAS 02250954 inhibits substrate phosphorylation. (A-B) The promiscuous inhibitor Compound C inhibits substrate phosphorylation. * p≦0.05, compared to DMSO control.

FIG. 12. Two analogs dose-dependently protect p-AMPK from dephosphorylation in the presence of PP2C.

FIG. 13. Two analogs modulate p-ACC levels in serum-starved HEK cells. (A) Western blots of cell lysates. The p-ACC bands for STK823366-treated cells are boxed. (B) Ratios of phospho-protein (normalized to loading control) to total protein (normalized to loading control). Data points are mean±σ and were scaled so that the ratio for DMSO=1.00. One of the 2 data points for 100-196 was discarded because the t-ACC signal could not be accurately quantified.

FIG. 14. STK823366 decreases p-ACC levels in serum-treated HEK cells. (A) Western blots of cell lysates. The p-ACC bands for STK823366-treated cells are boxed. (B) Ratios of phospho-protein (normalized to loading control) to total protein (normalized to loading control). Data points are mean±σ and were scaled so that the ratio for DMSO=1.00.

FIG. 15. BAS 02250954 does not increase p-ACC levels in pre-conditioned HEK cells. (A) Western blots of cell lysates. (B) Ratios of phospho-protein (normalized to loading control) to total protein (normalized to loading control). Data points are mean±σ and were scaled so that the ratio for DMSO=1.00. One of the 2 data points for DMSO was discarded because the p-AMPK signal could not be accurately quantified.

### DETAILED DESCRIPTION

The present invention is further described below in greater detail. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof. Further, all patent and patent application references referred to in this patent application are hereby incorporated by reference in their entirety as if set forth fully herein. Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an” or “the” can mean one or more than one. Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a compound) and the like, is meant to encompass variations of ±20%, ±10%, ±5%, ±1%, ±0.5%, or even ±0.1% of the specified amount.

“H” refers to a hydrogen atom. “C” refers to a carbon atom. “N” refers to a nitrogen atom. “O” refers to an oxygen atom. “S” refers to a sulfur atom.

“F” refers to a fluorine atom. “Cl” refers to a chlorine atom. “Br” refers to a bromine atom. “I” refers to an iodine atom. Fluorine, chlorine, bromine and iodine, along with astatine, constitute a “halogen” or “halo.”

“Alkyl”, as used herein, refers to a straight or branched chain hydrocarbon containing from 1 or 2 to 10 or 20 or more carbon atoms (e.g., C2, C3, C4, C5, C6, C7, C8, C9, C10, C11, C12, C13, C14, C15, etc.). In some embodiments the alkyl can be a lower alkyl. “Lower alkyl” refers to straight or branched chain alkyl having from 1 to 3, or from 1 to 5, or from 1 to 8 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. In some embodiments, alkyl groups as described herein are optionally substituted (e.g., from 1 to 3 or 4 times) with independently selected, but not limited to, H, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol.

As generally understood by those of ordinary skill in the art, “saturation” refers to the state in which all available valence bonds of an atom (e.g., carbon) are attached to other atoms. Similarly, “unsaturation” refers to the state in which not all the available valence bonds are attached to other atoms; in such compounds the extra bonds usually take the form of double or triple bonds (usually with carbon). For example, a carbon chain is “saturated” when there are no double or triple bonds present along the chain or directly connected to the chain (e.g., a carbonyl), and is “unsaturated” when at least one double or triple bond is present along the chain or directly connected to the chain (e.g., a carbonyl). Further, the presence or absence of a substituent depending upon chain saturation will be understood by those of ordinary skill in the art to depend upon the valence requirement of the atom or atoms to which the substituent binds (e.g., carbon). “Alkenyl”, as used herein, refers to a straight or branched chain hydrocarbon containing from 2 to 10 or 20 or more carbons, and containing at least one carbon-carbon double bond, formed structurally, for example, by the replacement of two hydrogens and having either E or Z regiochemistry and combinations thereof. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. In some embodiments, alkenyl groups as described herein are optionally substituted (e.g., from 1 to 3 or 4 times) with independently selected, but not limited to, H, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol.

“Alkynyl”, as used herein, refers to a straight or branched chain hydrocarbon group containing from 2 to 10 or 20 or more carbon atoms, and containing at least one carbon-carbon triple bond. Representative examples of alkynyl include, but are not limited, to acetylenyl, 1-propynyl, 2-propynyl, 1-butynyl, 2-butynyl, 2-pentynyl, and the like. In some embodiments, alkynyl groups as described herein are optionally substituted (e.g., from 1 to 3 or 4 times) with independently selected, but not limited to, H, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol.

The term “cycloalkyl”, as used herein, refers to a saturated or unsaturated cyclic hydrocarbon group containing from 3 to 8 carbons or more. Representative examples of cycloalkyl include cyclopropyl, cyclobutyl, cyclobutenyl, cyclopentyl, cyclohexyl, cycloheptyl, and cyclooctyl. In some embodiments, cycloalkyl groups as described herein are optionally substituted (e.g., from 1 to 3 or 4 times) with independently selected, but not limited to, H, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol.

“Heterocyclo”, “heterocyclic” and “heterocycle” as used herein, refers to a monocyclic, bicyclic or tricyclic ring system. Monocyclic heterocycle ring systems are exemplified by any 3, 4, 5 or 6 membered ring containing 1, 2, 3, or 4 heteroatoms independently selected from the group consisting of: O, N, and S. The 5 member ring has from 0 to 2 double bonds, and the 6 member ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiomorpholine sulfoxide, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, thiopyranopyridine, and the like. In some embodiments, heterocyclo groups as described herein are optionally substituted (e.g., from 1 to 3 or 4 times) with independently selected, but not limited to, H, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol.

“Aryl” as used herein refers to a ring system having one or more aromatic rings. Representative examples of aryl include azulenyl, indanyl, indenyl, naphthyl, phenyl, tetrahydronaphthyl, and the like. The aryl groups of this invention can be optionally substituted with 1, 2, 3, 4, 5, 6 or 7 substituents independently selected from, but not limited to, H, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol.

“Heteroaryl” means a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolinyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from the group consisting of: O, N, and S. The heteroaryl group, including each heteroatom, can be unsubstituted or substituted with from 1 to 4 suitable substituents, as chemically feasible. For example, the heteroatom S may be substituted with one or two oxo groups, which may be shown as ═O. In some embodiments, heteroaryl groups as described herein are optionally substituted (e.g., from 1 to 3 or 4 times) with independently selected H, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol.

An “acid” is a compound that can act as a proton donor or electron pair acceptor, and thus can react with a base. The strength of an acid corresponds to its ability or tendency to lose a proton. A “strong acid” is one that completely dissociates in water. Examples of strong acids include, but are not limited to, hydrochloric acid (HCl), hydroiodic acid (HI), hydrobromic acid (HBr), perchloric acid (HClO₄), nitric acid (HNO₃), sulfuric acid (H₂SO₄), etc. A “weak” or “mild” acid, by contrast, only partially dissociates, with both the acid and the conjugate base in solution at equilibrium. Examples of mild acids include, but are not limited to, carboxylic acids such as acetic acid, citric acid, formic acid, gluconic acid, lactic acid, oxalic acid, tartaric acid, ethylenediaminetetraacetic acid (EDTA), etc.

An “acid halide” as used herein refers to an organic functional group having a carbonyl group (C═O) linked to a halogen.

An “acyl group” is intended to mean a group —C(O)R, where R is a suitable substituent (for example, an acyl group may be an acetyl group (—C(O)CH₃), a propionyl group, a butyroyl group, a benzoyl group, or an alkylbenzoyl group).

“Aliphatic” is an acyclic or cyclic, non-aromatic carbon compound.

“Alkoxy”, as used herein, refers to an alkyl, alkenyl, alkynyl, aryl, cycloalkyl, heterocyclo, or heteroaryl group, as defined herein, appended to the parent molecular moiety through an oxy group, as defined herein. Representative examples of alkoxy include, but are not limited to, methoxy, ethoxy, propoxy, phenoxy, 2-propoxy, butoxy, tert-butoxy, pentyloxy, hexyloxy and the like. In some embodiments, alkoxy groups as described herein are optionally substituted (e.g., from 1 to 3 or 5 times) with independently selected, but not limited to, H, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol.

“Amidino” as used herein, refers to the —C(═NH)NH₂ moiety. “Optionally substituted” amidino refers to the NH and NH₂ groups wherein none, one, two or three of the hydrogens is replaced by a suitable substituent as described herein, such as alkyl, alkenyl, alkynyl, cycloalkyl, heterocyclo, aryl, heteroaryl, alkoxy, carbonyl, carboxy, etc.

An “amine” or “amino” is intended to mean the group —NH₂. “Optionally substituted” amines refers to —NH₂ groups wherein none, one or two of the hydrogens is replaced by a suitable substituent as described herein, such as alkyl, alkenyl, alkynyl, cycloalkyl, heterocyclo, aryl, heteroaryl, alkoxy, carbonyl, carboxy, etc. In some embodiments, one or two of the hydrogens are optionally substituted with independently selected, but not limited to, halo, hydroxy, acyl, alkyl, alkenyl, alkynyl, cycloalkyl, heterocyclo, aryl, heteroaryl, alkoxy, amino, amide, thiol, ester, sulfone, sulfoxide, oxo, oxy, nitro, carbonyl, carboxy, amino acid and peptide. Disubstituted amines may have substituents that are bridging, i.e., form a heterocyclic ring structure that includes the amine nitrogen.

An “amine”, “organic amine”, “amine base” or “organic amine base” as used herein refers to an organic compound having a basic nitrogen atom (R—NR′R″), and may be a primary (R—NH₂), secondary (R—NHR′) or tertiary (R—NR′R″) amine. R, R′ and R″ may be independently selected from the group consisting of alkyl (e.g., cycloalkyl), aryl and heteroaryl, which groups may be optionally substituted, or R and R′, R and R″ and/or R′ and R″, when present, may also combine to form cyclic or heteroalicyclic ring. In some embodiments the amine is aromatic. Examples of aromatic amines include, but are not limited to, pyridine, pyrimidine, quinoline, isoquinolines, purine, pyrrole, imidazole, and indole. The aromatic amines may be substituted or unsubstituted. Examples of amines include, but are not limited to, triethylamine, pyridine, dimethylaminopyridine, N-methylmorpholine, Hunig's base (N,N-diisopropylethylamine), and 1,8-diazabicyclo[5.4.0]undec-7-ene (DBU).

An “amide” as used herein refers to an organic functional group having a carbonyl group (C═O) linked to a nitrogen atom (N), or a compound that contains this group, generally depicted as:

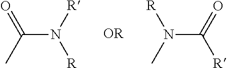

wherein, R and R′ can independently be any covalently-linked atom or atoms, for example, H, halo, hydroxy, acyl, alkyl, alkenyl, alkynyl, cycloalkyl, heterocyclo, aryl, heteroaryl, alkoxy, amino, amide, sulfone, sulfoxide, carboxy, amino acid and peptide.

An “amide coupling agent” is an agent that may be used to couple a nitrogen and carboxyl group to form an amide, typically by activating the carboxyl group. Examples of amide coupling agents include, but are not limited to, carbodiimides such as N,N′-dicyclohexylcarbodiimide (DCC), N-(3-dimethylaminopropyl)-N′-ethylcarbodiimide (EDC) or N-(3-dimethylaminopropyl)-N-ethylcarbodiimide hydrochloride (EDAC), N,N′-diisopropylcarbodiimide (DIC); imidazoliums such as 1,1′-carbonyldiimidazole (CDI), 1,1′-carbonyl-di-(1,2,4-triazole) (CDT); uronium or guanidinium salts such as O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluorophosphate (HBTU), and O-(benzotriazol-1-yl)-N,N,N′,N′-tetramethyluronium tetrafluoroborate (TBTU); phosphonium salts such as benzotriazol-1-yloxy)tris(dimethylamino)phosphonium hexafluorophosphate (BOP or Castro's reagent), (benzotriazol-1-yloxy)tripyrrolidinophosphonium hexafluorophosphate (PyBOP®, Merck KGaA, Germany), 7-azabenxotriazol-1-yloxy)tripynolidinophosphonium hexafluorophosphate (PyAOP); alkyl phosphonic acid anhydrides such as T3P® (Archimica, Germany), etc. In another embodiment the carboxyl group may be activated by forming an acid halide or acid anhydride with an agent including but not limited to thionyl chloride, phosphorus pentachloride, and phosphorus trichloride.

“Amino acid sidechain” as used herein refers to any of the 20 commonly known groups associated with naturally-occurring amino acids, or any natural or synthetic homologue thereof. An “amino acid” includes the sidechain group, the amino group, alpha-carbon atom, and carboxy groups, as commonly described in the art. Examples of amino acids include glycine, and glycine that is substituted with a suitable substituent as described herein, such as alkyl, alkenyl, alkynyl, cycloalkyl, heterocyclo, aryl, heteroaryl, alkoxy, carbonyl, carboxy, etc., or a pharmaceutically acceptable salt or prodrug thereof. For example, “Histidine” is one of the 20 most commonly known amino acids found naturally in proteins. It contains a —(CH₂)-imidazole side chain substituent. Other examples of naturally-occurring amino acids include lysine, arginine, aspartic acid, glutamic acid, asparagine, glutamine, serine, threonine, tyrosine, alanine, valine, leucine, isoleucine, phenylalanine, methionine, tryptophan, and cysteine. Also included in the definitions of “amino acid sidechain” and “amino acid” is proline, which is commonly included in the definition of an amino acid, but is technically an imino acid. As used in this application, both the naturally-occurring L- and the non-natural D-amino acid enantiomers are included. The single letter code for amino acids is A (Ala), C (Cys), D (Asp), E (Glu), F (Phe), G (Gly), H (His), I (Ile), K (Lys), L (Leu), M (Met), N (Asn), P (Pro), Q (Gln), R (Arg), S (Ser), T (Thr), V (Val), W (Trp), and Y (Tyr).

“Aqueous” is a solution in which water is the dissolving medium, or solvent. An “aqueous base” is a base in water. An “aqueous acid” is an acid in water.

“Araalkyl”, as used herein, refers to an alkyl group that has an aryl group appended thereto, for example benzyl and naphthylmethyl groups.

“Azido”, as used herein, refers to the —N₃ functional group.

A “base” is a compound that can accept a proton (hydrogen ion) or donate an electron pair. A base may be organic (e.g., DBU, cesium carbonate, etc.) or inorganic. A “strong base” as used herein is a compound that is capable of deprotonating very weak acids. Examples of strong bases include, but are not limited to, hydroxides, alkoxides, and ammonia.

“Carbonate”, as used herein refers to a —O(CO₂)R functional group wherein R is for example, alkyl, alkenyl, alkynyl, cycloalkyl, heterocyclo, aryl and heteroaryl that may be optimally substituted.

“Carbonyl” is a functional group having a carbon atom double-bonded to an oxygen atom (C═O).

“Carboxy” and “carboxylic acid” as used herein refers to a —COOH functional group, also written as —CO₂H or —(C═O)—OH.

“Cyano” refers to the group —C≡N, or —CN.

“Ester” as used herein refers to a —COOR functional group, also written as —CO₂R or —(C═O)—OR wherein, R is for example, alkyl, alkenyl, alkynyl, cycloalkyl, heterocyclo, aryl and heteroaryl that may be optimally substituted.

“Form a ring” as used herein with respect to two substituents, e.g., R⁷ and R⁸ together forming a ring, refers to the two groups being linked together via one or more atoms (e.g., carbon) to form ring atoms making up a cycloalkyl, heterocyclo, aryl or heteroaryl as described herein. Rings may be part of a monocyclic, bicyclic or tricyclic moiety, each of such ring(s) being a saturated or unsaturated member of the monocyclic, bicyclic or tricyclic moiety.

“Formylated”, as used herein, refers to a chemical reaction that introduces a formyl group (methanoyl, —CHO) into an organic molecule.

“Formyl” or “formyl group”, as used herein, refers to a —CHO moiety.

“Hydroxy”, as used herein, refers to an HO— moiety.

A “hydroxide” is the commonly known anion HO⁻, or a salt thereof (typically an alkali metal or alkaline earth metal salt thereof). Examples of hydroxides include, but are not limited to, sodium hydroxide (NaOH), potassium hydroxide (KOH), lithium hydroxide (LiOH), and calcium hydroxide (Ca(OH)₂).

An “inorganic” compound is a compound not containing carbon.

The term “oxo”, as used herein, refers to a ═O moiety.

The term “oxy”, as used herein, refers to a —O— moiety.

“Nitro” refers to the organic compound functional group —NO₂.

A “thiol” or “mercapto” refers to an —SH group, its tautomer ═S or —SR wherein, R is for example alkyl, alkenyl, alkynyl, cycloalkyl, heterocyclo, aryl and heteroaryl that may be optimally substituted.

A “sulfone” as used herein refers to a sulfonyl functional group, generally depicted as:

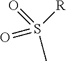

wherein, R can be any covalently-linked atom or atoms, for example, H, halo, hydroxy, alkyl, alkenyl, alkynyl, cycloalkyl, heterocyclo, aryl, heteroaryl, alkoxy, amino, amino acid and peptide.

A “sulfoxide” as used herein refers to a sulfinyl functional group, generally depicted as:

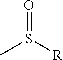

wherein, R can be any covalently-linked atom or atoms, for example, H, halo, hydroxy, alkyl, alkenyl, alkynyl, cycloalkyl, heterocyclo, aryl, heteroaryl, alkoxy, amino, amino acid and peptide.

The term “optionally substituted” indicates that the specified group is either unsubstituted or substituted by one or more suitable substituents. A “substituent” is an atom or group which takes the place of a hydrogen atom on the parent chain or cycle of an organic molecule, examples include, but are not limited to, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol. In some embodiments, the substituent may be further substituted. For example, an atom or group which takes the place of a hydrogen atom on the substituent; examples include, but are not limited to, H, acyl, alkyl, alkenyl, alkoxy, alkynyl, amidino, amino, amino acid, amide, aryl, azido, carbonate, carbonyl, carboxy, cyano, cycloalkyl, ester, formyl, halo, heterocyclo, heteroaryl, hydroxy, nitro, oxo, oxy, peptide, sulfone, sulfoxide, and thiol.

An “organic” compound as used herein is a compound that contains carbon.

An “organic solvent” is a compound containing carbon that is useful as a solvent. Examples of organic solvents include, but are not limited to, acid amides such as N,N-dimethylformamide and N,N-dimethylacetamide; alcohols such as ethanol, methanol, isopropanol, amyl alcohol, ethylene glycol, propylene glycol, 1-butanol, butyl carbitol acetate and glycerin; aliphatic hydrocarbons such as hexane and octane; aromatic hydrocarbons such as toluene, xylenes and benzene; ketones such as acetone, methyl ethyl ketone and cyclohexanone; halogenated hydrocarbons such as dichloromethane, chlorobenzene and chloroform; esters such as ethyl acetate, amyl acetate and butyl acetate; ethers such as tetrahydrofuran, 2-methyltetrahydrofuran, 1,4-dioxane, tert-butyl methyl ether, diethyl ether and ethylene glycol dimethyl ether; nitriles such as acetonitrile; and sulfoxides such as dimethylsulfoxide.

An “oxidizing agent” is an agent useful to oxidize a compound, whereby the compound loses electrons or increases its oxidation state. Examples include, but are not limited to, oxygen, ozone, organic peroxides such as hydrogen peroxide, halogens such as fluorine or chlorine, or halogen compounds such as chlorite, chlorate or perchlorate, nitrate compounds such as nitric acid, a sulfuric acid or persulfuric acid, hypohalite compounds such as hypochlorite and sodium hypochlorite (NaOCl), hexavalent chromium compounds such as chromic and dichromic acids and chromium trioxide, pyridinium chlorochromate and chromate/dichromate compounds, permanganate compounds, sodium perborate, nitrous oxide, silver oxide, osmium tetroxide, Tollens' reagent, and 2,2′-dipyridyldisulfide.

A “peptide” is a linear chain of amino acids covalently linked together, typically through an amide linkage, and contains from 1 or 2 to 10 or 20 or more amino acids, and is also optionally substituted and/or branched.

“Protecting group” as used herein, is meant that a particular functional moiety, e.g., O, S, or N, is temporarily blocked so that a reaction can be carried out selectively at another reactive site in a multifunctional compound. For example, in certain embodiments, as detailed herein, certain exemplary oxygen protecting groups are utilized. Oxygen protecting groups include, but are not limited to, groups bonded to the oxygen to form an ether, such as methyl, substituted methyl (e.g., Trt (triphenylmethyl), MOM (methoxymethyl), MTM (methylthiomethyl), BOM (benzyloxymethyl), PMBM or MPM (p-methoxybenzyloxymethyl)), substituted ethyl (e.g., 2-(trimethylsilyl)ethyl), benzyl, substituted benzyl (e.g., para-methoxybenzyl), silyl (e.g., TMS (trimethylsilyl), TES (triethylsilyl), TIPS (triisopropylsilyl), TBDMS (t-butyldimethylsilyl), TBDPS (t-butyldiphenylsilyl), 2-trimethylsilylprop-2-enyl, t-butyl, tetrahydropyranyl, allyl, etc.

Embodiments of the present invention relate to methods for identifying a compound that modulates adenosine monophosphate-activated protein kinase (AMPK) for the manufacture of a diagnostic or therapeutic agent, comprising, consisting essentially of or consisting of (a) contacting a sample comprising AMPK with a luminescent agent known to bind AMPK; (b) contacting the sample from (a) with a compound of interest; and (c) comparing the luminescence in the sample prior to contacting the sample with the compound of interest to the luminescence in the sample after contacting the sample with the compound of interest, that is, comparing the luminescence in (a) with the luminescence in (b), wherein a decrease in luminescence after contacting the sample with the compound of interest indicates that the compound of interest is a modulator of AMPK. The methods may be in vitro or in vivo methods.

As discussed above, 5′ adenosine monophosphate-activated protein kinase, adenosine monophosphate-activated protein kinase or “AMPK” is a conserved heterotrimeric serine-threonine kinase that regulates anabolic and catabolic pathways in eukaryotes. The mammalian AMPK trimer is composed of one catalytic subunit (α₁ or α₂) and two regulatory subunits (β₁ or β₂ and γ₁, γ₂, or γ₃) (Carling D, Thornton C, Woods A, Sanders M J. AMP-activated protein kinase: new regulation, new roles? Biochem J 2012; 445(1):11-27; Oakhill J S, Scott J W, Kemp B E. AMPK functions as an adenylate charge-regulated protein kinase. Trends Endocrinol Metab 2012; 23(3):125-3). AMPK-γ has four potential nucleotide binding pockets, of which one (Site 2) is constitutively unoccupied (Xiao B, Heath R, Saiu P, et al. Structural basis for AMP binding to mammalian AMP-activated protein kinase. Nature 2007; 449(7161):496-500). Previous studies have identified several modes of AMPK regulation: (1) Phosphorylation of threonine 172 (T172) on AMPK-α activates AMPK; (2) Binding of AMP to AMPK-γ (Site 1) allosterically increases activity of p-AMPK by 2 to 5-fold; and (3) Binding of AMP or ADP to AMPK-γ (Site 3) inhibits dephosphorylation of p-T172 by phosphatases (Oakhill J S, Scott J W, Kemp B E. AMPK functions as an adenylate charge-regulated protein kinase. Trends Endocrinol Metab 2012; 23(3):125-32;Xiao B, Sanders M J, Underwood E, et al. Structure of mammalian AMPK and its regulation by ADP. Nature 2011; 472(7342):230-3). AMP and ADP have also been shown to promote the phosphorylation of AMPK, but only when AMPK-β is myristoylated (Carling D, Thornton C, Woods A, Sanders M J. AMP-activated protein kinase: new regulation, new roles? Biochem J 2012; 445(1):11-27; Oakhill J S, Chen Z P, Scott J W, et al. beta-Subunit myristoylation is the gatekeeper for initiating metabolic stress sensing by AMP-activated protein kinase (AMPK). Proc Natl Acad Sci USA 2010; 107(45):19237-41; Chen L, Wang J, Zhang Y Y, et al. AMP-activated protein kinase undergoes nucleotide-dependent conformational changes. Nat Struct Mol Biol 2012; 19(7):716-8; Oakhill J S, Steel R, Chen Z P, et al. AMPK is a direct adenylate charge-regulated protein kinase. Science 2011; 332(6036):1433-5). Site 4 has been proposed to bind AMP in a non-exchangeable manner (Xiao B, Heath R, Saiu P, et al. Structural basis for AMP binding to mammalian AMP-activated protein kinase. Nature 2007; 449(7161):496-500; Xiao B, Sanders M J, Underwood E, et al. Structure of mammalian AMPK and its regulation by ADP. Nature 2011; 472(7342):230-3). Most AMPK researchers agree that AMPK-γ has two primary regulatory nucleotide-binding sites that, together, allosterically increase and sustain activity of p-AMPK (Xiao B, Sanders M J, Underwood E, et al. Structure of mammalian AMPK and its regulation by ADP. Nature 2011; 472(7342):230-3; Chen L, Wang J, Zhang Y Y, et al. AMP-activated protein kinase undergoes nucleotide-dependent conformational changes. Nat Struct Mol Biol 2012; 19(7):716-8; Oakhill J S, Steel R, Chen Z P, et al. AMPK is a direct adenylate charge-regulated protein kinase. Science 2011; 332(6036):1433-5). As used herein, AMPK refers to a full-length AMPK, a truncated AMPK, or combination thereof as noted. Human and rodent nucleic acid sequence data for AMPK can be found in Table 1 below.

TABLE 1
AMPK human and rodent nucleic acid sequence data.

>Rattus norvegicus AMPK-gamma1 (330 aa)
MESVAAESAPAPENEHSQETPESNSSVYTTFMKSHRCYDLIPTSSKLVVFDTSLQVKKAFFALVTNGV
RAAPLWDSKKQSFVGMLTITDFINILHRYYKSALVQIYELEEHKIETWREVYLQDSFKPLVCISPNASLF
DAVSSLIRNKIHRLPVIDPESGNTLYILTHKRILKFLKLFITEFPKPEFMSKSLEELQIGTYANIAMVRTTT
PVYVALGIFVQHRVSALPVVDEKGRVVDIYSKFDVINLAAEKTYNNLDVSVTKALQHRSHYFEGVLKC
YLHETLEAIINRLVEAEVHRLVVVDEHDVVKGIVSLSDILQALVLTGGEKKP
>Rattus norvegicus AMPK-beta1 (270 aa)
MGNTSSERAALERQAGHKTPRRDSSGGTKDGDRPKILMDSPEDADIFHTEEMMPEKEEFLAWQHD
LEVNEKAPAQARPTVFRVVTGGGKEVYLSGSFNNWSKLPLTRSQNNFVAILDLPEGEHQYKFFVDGQ
WTHDPSEPIVTSQLGTVNNIIQVKKTDFEVFDALMVDSQKCSDVSELSSSPPGPYHQEPYISKPEERF
KAPPILPPHLLQVILNKDTGISCDPALLPEPNHVMLNHLYALSIKDGVMVLSATHRYKKKYVTTLLYKPI
>Rattus norvegicus AMPK-alpha1 (548 aa, full-length)
MAEKQKHDGRVKIGHYILGDTLGVGTFGKVKVGKHELTGHKVAVKILNRQKIRSLDVVGKIRREIQNLK
LFRHPHIIKLYQVISTPSDIFMVMEYVSGGELFDYICKNGRLDEKESRRLFQQILSGVDYCHRHMVVHR
DLKPENVLLDAHMNAKIADFGLSNMMSDGEFLRTSCGSPNYAAPEVISGRLYAGPEVDIWSSGVILYA
LLCGTLPFDDDHVPTLFKKICDGIFYTPQYLNPSVISLLKHMLQVDPMKRATIKDIREHEWFKQDLPKY
LFPEDPSYSSTMIDDEALKEVCEKFECSEEEVLSCLYNRNHQDPLAVAYHLIIDNRRIMNEAKDFYLAT
SPPDSFLDDHHLTRPHPERVPFLVAETPRARHTLDELNPQKSKHQGVRKAKWHLGIRSQSRPNDIMA
EVCRAIKQLDYEWKVVNPYYLRVRRKNPVTSTFSKMSLQLYQVDSRTYLLDFRSIDDEITEAKSGTAT
PQRSGSISNYRSCQRSDSDAEAQGKPSEVSLTSSVTSLDSSPVDVAPRPGSHTIEFFEMCANLIKILA
Q
>Rattus norvegicus AMPK-alpha1, Truncated (aa 396-548)
WHLGIRSQSRPNDIMAEVCRAIKQLDYEWKWNPYYLRVRRKNPVTSTFSKMSLQLYQVDSRTYLLD
FRSIDDEITEAKSGTATPQRSGSISNYRSCQRSDSDAEAQGKPSEVSLTSSVTSLDSSPVDVAPRPGS
HTIEFFEMCANLIKILAQ
>Rattus norvegicus AMPK-alpha1, Kinase Domain (aa 22-290)
VKIGHYILGDTLGVGTFGKVKVGKHELTGHKVAVKILNRQKIRSLDVVGKIRREIQNLKLFRHPHIIKLYQ
VISTPSDIFMVMEYVSGGELFDYICKNGRLDEKESRRLFQQILSGVDYCHRHMVVHRDLKPENVLLDA
HMNAKIADFGLSNMMSDGEFLRTSCGSPNYAAPEVISGRLYAGPEVDIWSSGVILYALLCGTLPFDDD
HVPTLFKKICDGIFYTPQYLNPSVISLLKHMLQVDPMKRATIKDIREHEWFKQDLPKYLFPE
>Rattus norvegicus AMPK-alpha2
MAEKQKHDGRVKIGHYVLGDTLGVGTFGKVKIGEHQLTGHKVAVKILNRQKIRSLDVVGKIKREIQNLK
LFRHPHIIKLYQVISTPTDFFMVMEYVSGGELFDYICKHGRVEEVEARRLFQQILSAVDYCHRHMVVH
RDLKPENVLLDAQMNAKIADFGLSNMMSDGEFLRTSCGSPNYAAPEVISGRLYAGPEVDIWSCGVIL
YALLCGTLPFDDEHVPTLFKKIRGGVFYIPEYLNRSIATLLMHMLQVDPLKRATIKDIREHEWFKQDLPS
YLFPEDPSYDANVIDDEAVKEVCEKFECTESEVMNSLYSGDPQDQLAVAYHLIIDNRRIMNQASEFYL
ASSPPTGSFMDDSAMHIPPGLKPHPERMPPLIADSPKARCPLDALNTTKPKSLAVKKAKWHLGIRSQ
SKPYDIMAEVYRAMKQLDFEWKVVNAYHLRVRRKNPVTGNYVKMSLQLYLVDNRSYLLDFKSIDDEV
VEQRSGSSTPQRSCSAAGLHRPRSSVDSSTAENHSLSGSLTGSLTGSTLSSASPRLGSHTMDFFEM
CASLITALAR
>Rattus norvegicus AMPK-alpha2 Kinase Domain (aa 11-278)
VKIGHYVLGDTLGVGTFGKVKIGEHQLTGHKVAVKILNRQKIRSLDVVGKIKREIQNLKLFRHPHIIKLYQ
VISTPTDFFMVMEYVSGGELFDYICKHGRVEEVEARRLFQQILSAVDYCHRHMVVHRDLKPENVLLD
AQMNAKIADFGLSNMMSDGEFLRTSCGSPNYAAPEVISGRLYAGPEVDIWSCGVILYALLCGTLPFDD
EHVPTLFKKIRGGVFYIPEYLNRSIATLLMHMLQVDPLKRATIKDIREHEWFKQDLPSYLFP
>Rattus norvegicus AMPK-alpha2 truncated (aa 402-552)
WHLGIRSQSKPYDIMAEVYRAMKQLDFEWKVVNAYHLRVRRKNPVTGNYVKMSLQLYLVDNRSYLL
DFKSIDDEVVEQRSGSSTPQRSCSAAGLHRPRSSVDSSTAENHSLSGSLTGSLTGSTLSSASPRLGS
HTMDFFEMCASLITALAR
>Rattus norvegicus AMPK-beta
2MGNTTSERVSGERHGAKAARAEGGGHGPGKEHKIMVGSTDDPSVFSLPDSKLPGDKEFVPWQQD
LDDSVKPTQQARPTVIRWSEGGKEVFISGSFNNWSTKIPLIKSHNDFVAILDLPEGEHQYKFFVDGQW
VHDPSEPVVISQLGTINNLIHVKKSDFEVFDALKLDSMESSETSCRDLSSSPPGPYGQEMYVFRSEE
RFKSPPILPPHLLQVILNKDTNISCDPALLPEPNHVMLNHLYALSTKDSVMVLSATHRYKKKYVTTLLYK
PI
>Rattus norvegicus AMPK gamma
2MLEKLEFQEEEDSESGVYMRFMRSHKCYDIVPTSSKLVVFDTTLQVKKAFFALVANGVRAAPLWES
KKQSFVGMLTITDFINILHRYYKSPMVQIYELEEHKIETVVRELYLQETFKPLVNISPDASLFDAVYSLIKN
KIHRLPVIDIDISGNALYILTHKRILKFLQLFMSDMPKPAFMKQNLDELGIGTYHNIAFIHPNTPIIKALNI
FVERRISALPVVDESGKVVDIYSKFDVINLAAEKTYNNLDITVTQALQHRSQYFEGVVKCSKLETLETIVDR
IVRAEVHRLVVVNEADSIVGIISLSDILQALILTPAGAKQKETETE
>Human AMPK-gamma1
METVISSDSSPAVENEHPQETPESNNSVYTSFMKSHRCYDLIPTSSKLVVFDTSLQVKKAFFALVING
VRAAPLWDSKKQSFVGMLTITDFINILHRYYKSALVQIYELEEHKIETWREVYLQDSFKPLVCISPNASL
FDAVSSLIRNKIHRLPVIDPESGNTLYILTHKRILKFLKLFITEFPKPEFMSKSLEELQIGTYANIAMVRTT
TPVYVALGIFVQHRVSALPVVDEKGRVVDIYSKFDVINLAAEKTYNNLDVSVTKALQHRSHYFEGVLK
CYLHETLETIINRLVEAEVHRLVVVDENDVVKGIVSLSDILQALVLTGGEKKP
>human gamma 2 isoform
aMGSAVMDTKKKKDVSSPGGSGGKKNASQKRRSLRVHIPDLSSFAMPLLDGDLEGSGKHSSRKVDS
PFGPGSPSKGFFSRGPQPRPSSPMSAPVRPKTSPGSPKTVFPFSYQESPPRSPRRMSFSGIFRSSS
KESSPNSNPATSPGGIRFFSRSRKTSGLSSSPSTPTQVTKQHTFPLESYKHEPERLENRIYASSSPPD
TGQRFCPSSFQSPTRPPLASPTHYAPSKAAALAAALGPAEAGMLEKLEFEDEAVEDSESGVYMRFM
RSHKCYDIVPTSSKLVVFDTTLQVKKAFFALVANGVRAAPLWESKKQSFVGMLTITDFINILHRYYKSP
MVQIYELEEHKIETVVRELYLQETFKPLVNISPDASLFDAVYSLIKNKIHRLPVIDPISGNALYILTHKRIL
KFLQLFMSDMPKPAFMKQNLDELGIGTYHNIAFIHPDTPIIKALNIFVERRISALPVVDESGKVVDIYSKFD
VINLAAEKTYNNLDITVTQALQHRSQYFEGVVKCNKLEILETIVDRIVRAEVHRLVVVNEADSIVGIISLS
DILQALILTPAGAKQKETETE
>human gamma 2 isoform
bMLEKLEFEDEAVEDSESGVYMRFMRSHKCYDIVPTSSKLVVFDTTLQVKKAFFALVANGVRAAPLW
ESKKQSFVGMLTITDFINILHRYYKSPMVQIYELEEHKIETWRELYLQETFKPLVNISPDASLFDAVYSLI
KNKIHRLPVIDPISGNALYILTHKRILKFLQLFMSDMPKPAFMKQNLDELGIGTYHNIAFIHPDTPIIKALN
IFVERRISALPVVDESGKVVDIYSKFDVINLAAEKTYNNLDITVTQALQHRSQYFEGVVKCNKLEILETIV
DRIVRAEVHRLVVVNEADSIVGIISLSDILQALILTPAGAKQKETETE
>human gamma 2 isoform
cMPLLDGDLEGSGKHSSRKVDSPFGPGSPSKGFFSRGPQPRPSSPMSAPVRPKTSPGSPKTVFPFS
YQESPPRSPRRMSFSGIFRSSSKESSPNSNPATSPGGIRFFSRSRKTSGLSSSPSTPTQVTKQHTFP
LESYKHEPERLENRIYASSSPPDTGQRFCPSSFQSPTRPPLASPTHYAPSKAAALAAALGPAEAGML
EKLEFEDEAVEDSESGVYMRFMRSHKCYDIVPTSSKLVVFDTTLQVKKAFFALVANGVRAAPLWESK
KQSFVGMLTITDFINILHRYYKSPMVQIYELEEHKIETWRELYLQETFKPLVNISPDASLFDAVYSLIKNK
IHRLPVIDPISGNALYILTHKRILKFLQLFMSDMPKPAFMKQNLDELGIGTYHNIAFIHPDTPIIKALNIFV
ERRISALPVVDESGKVVDIYSKFDVINLAAEKTYNNLDITVTQALQHRSQYFEGVVKCNKLEILETIVDRIV
RAEVHRLVWNEADSIVGIISLSDILQALILTPAGAKQKETETE
>human gamma
3MEPGLEHALRRIPSWSSLGGSEHQEMSFLEQENSSSWPSPAVTSSSERIRGKRRAKALRVVTRQK
SVEEGEPPGQGEGPRSRPAAESTGLEATFPKTTPLAQADPAGVGTPPTGWDCLPSDCTASAAGSST
DDVELATEFPATEAWECELEGLLEERPALCLSPQAPFPKLGWDDELRKPGAQIYMRFMQEHTCYDA
MATSSKLVIFDTMLEIKKAFFALVANGVRAAPLWDSKKQSFVGMLTITDFILVLHRYYRSPLVQIYEIEQ
HKIETWREIYLQGCFKPLVSISPNDSLFEAVYTLIKNRIHRLPVLDPVSGNVLHILTHKRLLKFLHIFGSLL
PRPSFLYRTIQDLGIGTFRDLAVVLETAPILTALDIFVDRRVSALPWNECGQVVGLYSRFDVIHLAAQQ
TYNHLDMSVGEALRQRTLCLEGVLSCQPHESLGEVIDRIAREQVHRLVLVDETQHLLGVVSLSDILQA
LVLSPAGIDALGA
>Human AMPK-
beta1MGNTSSERAALERHGGHKTPRRDSSGGTKDGDRPKILMDSPEDADLFHSEEIKAPEKEEFLAW
QHDLEVNDKAPAQARPTVFRWMGGKEVYLSGSFNNWSKLPLTRSHNNFVAILDLPEGEHQYKFFV
DGQVVTHDPSEPIVTSQLGTVNNIIQVKKTDFEVFDALMVDSQKCSDVSELSSSPPGPYHQEPYVCKID
EERFRAPPILPPHLLQVILNKDTGISCDPALLPEPNHVMLNHLYALSIKDGVMVLSATHRYKKKYVVILL
YKPI
>Human AMPK-
beta2MGNTTSDRVSGERHGAKAARSEGAGGHAPGKEHKIMVGSTDDPSVFSLPDSKLPGDKEFVS
WQQDLEDSVKPTQQARPTVIRWSEGGKEVFISGSFNNWSTKIPLIKSHNDFVAILDLPEGEHQYKFFV
DGQVVVHDPSEPVVTSQLGTINNLIHVKKSDFEVFDALKLDSMESSETSCRDLSSSPPGPYGQEMYAF
RSEERFKSPPILPPHLLQVILNKDTNISCDPALLPEPNHVMLNHLYALSIKDSVMVLSATHRYKKKYVTT
LLYKPI
>Human AMPK-beta2 (truncated, aa 187-272)
GPYGQEMYAFRSEERFKSPPILPPHLLQVILNKDTNISCDPALLPEPNHVMLNHLYALSIKDSVMVLSA
THRYKKKYVTTLLYKPI
>Human AMPK-alpha1 (559 aa)
MRRLSSWRKMATAEKQKHDGRVKIGHYILGDTLGVGTFGKVKVGKHELTGHKVAVKILNRQKIRSLD
VVGKIRREIQNLKLFRHPHIIKLYQVISTPSDIFMVMEYVSGGELFDYICKNGRLDEKESRRLFQQILSG
VDYCHRHMVVHRDLKPENVLLDAHMNAKIADFGLSNMMSDGEFLRTSCGSPNYAAPEVISGRLYAG
PEVDIWSSGVILYALLCGTLPFDDDHVPTLFKKICDGIFYTPQYLNPSVISLLKHMLQVDPMKRATIKDI
REHEWFKQDLPKYLFPEDPSYSSTMIDDEALKEVCEKFECSEEEVLSQLYNRNHQDPLAVAYHLIIDN
RRIMNEAKDFYLATSPPDSFLDDHHLTRPHPERVPFLVAETPRARHTLDELNPQKSKHQGVRKAKVVH
LGIRSQSRPNDIMAEVCRAIKQLDYEWKVVNPYYLRVRRKNPVTSTYSKMSLQLYQVDSRTYLLDFR
SIDDEITEAKSGTATPQRSGSVSNYRSCQRSDSDAEAQGKSSEVSLTSSVTSLDSSPVDLTPRPGSH
TIEFFEMCANLIKILAQ
>Human AMPK-alpha1, Kinase Domain (aa 22-289)
VKIGHYILGDTLGVGTFGKVKVGKHELTGHKVAVKILNRQKIRSLDWGKIRREIQNLKLFRHPHIIKLYQ
VISTPSDIFMVMEYVSGGELFDYICKNGRLDEKESRRLFQQILSGVDYCHRHMVVHRDLKPENVLLDA
HMNAKIADFGLSNMMSDGEFLRTSCGSPNYAAPEVISGRLYAGPEVDIWSSGVILYALLCGTLPFDDD
HVPTLFKKICDGIFYTPQYLNPSVISLLKHMLQVDPMKRATIKDIREHEWFKQDLPKYLFP
>Human AMPK-alpha1, Truncated (aa 407-559)
WHLGIRSQSRPNDIMAEVCRAIKQLDYEWKVVNPYYLRVRRKNPVTSTYSKMSLQLYQVDSRTYLLD
FRSIDDEITEAKSGTATPQRSGSVSNYRSCQRSDSDAEAQGKSSEVSLTSSVTSLDSSPVDLTPRPG
SHTIEFFEMCANLIKILAQ
>Human AMPK-alpha2 (552 aa)
MAEKQKHDGRVKIGHYVLGDTLGVGTFGKVKIGEHQLTGHKVAVKILNRQKIRSLDVVGKIKREIQNLK
LFRHPHIIKLYQVISTPTDFFMVMEYVSGGELFDYICKHGRVEEMEARRLFQQILSAVDYCHRHMWH
RDLKPENVLLDAHMNAKIADFGLSNMMSDGEFLRTSCGSPNYAAPEVISGRLYAGPEVDIWSCGVILY
ALLCGTLPFDDEHVPTLFKKIRGGVFYIPEYLNRSVATLLMHMLQVDPLKRATIKDIREHEWFKQDLPS
YLFPEDPSYDANVIDDEAVKEVCEKFECTESEVMNSLYSGDPQDQLAVAYHLIIDNRRIMNQASEFYL
ASSPPSGSFMDDSAMHIPPGLKPHPERMPPLIADSPKARCPLDALNTTKPKSLAVKKAKWHLGIRSQ
SKPYDIMAEVYRAMKQLDFEWKVVNAYHLRVRRKNPVTGNYVKMSLQLYLVDNRSYLLDFKSIDDEV
VEQRSGSSTPQRSCSAAGLHRPRSSFDSTTAESHSLSGSLTGSLTGSTLSSVSPRLGSHTMDFFEM
CASLITTLAR
>Human AMPK-alpha2, Kinase Domain (aa 11-278)
VKIGHYVLGDTLGVGTFGKVKIGEHQLTGHKVAVKILNRQKIRSLDVVGKIKREIQNLKLFRHPHIIKLYQ
VISTPTDFFMVMEYVSGGELFDYICKHGRVEEMEARRLFQQILSAVDYCHRHMVVHRDLKPENVLLD
AHMNAKIADFGLSNMMSDGEFLRTSCGSPNYAAPEVISGRLYAGPEVDIWSCGVILYALLCGTLPFDD
EHVPTLFKKIRGGVFYIPEYLNRSVATLLMHMLQVDPLKRATIKDIREHEWFKQDLPSYLFP
>Human AMPK-alpha2, Truncated (aa 402-552)
WHLGIRSQSKPYDIMAEVYRAMKQLDFEWKVVNAYHLRVRRKNPVTGNYVKMSLQLYLVDNRSYLL
DFKSIDDEVVEQRSGSSTPQRSCSAAGLHRPRSSFDSTTAESHSLSGSLTGSLTGSTLSSVSPRLGS
HTMDFFEMCASLITTLAR

The full-length AMPK includes at least the functional portions of at least one of each of the catalytic and regulatory subunits. In some embodiments, the full-length AMPK includes at least the functional portion of all catalytic and regulatory subunits. In some embodiments, the full-length AMPK includes all subunits. In some embodiments, the full-length AMPK includes rat His-α₁, β₁, γ₁. In still other embodiments, a truncated AMPK is utilized. The truncated AMPK may include at least the functional portion of one of either a catalytic or regulatory subunit. In some embodiments, the truncated AMPK is at least a portion or fragment of the regulatory subunit. In particular embodiments, the regulatory fragment of AMPK lacks the kinase domain. In some embodiments, the heterotrimeric truncated AMPK (in contrast to full-length AMPK trimer) lacks the canonical ATP-binding site found throughout the kinome and is termed the AMPK “regulatory fragment.” In some embodiments, the truncated AMPK is rat His-α₁, 396-548; human β₂, 187-272; rat γ₁. As used herein, “portion” or “fragment” are used interchangeably and refers to less than the whole of the structure that substantially retains at least one biological activity normally associated with that molecule, protein or polypeptide. In particular embodiments, the “fragment” or “portion” substantially retains all of the activities possessed by the unmodified protein. By “substantially retains” biological activity, it is meant that the protein retains at least about 50%, 60%, 75%, 85%, 90%, 95%, 97%, 98%, 99%, or more, of the biological activity of the native protein (and can even have a higher level of activity than the native protein).

According to embodiments of the present invention, the sample includes AMPK as described above and a luminescent agent. The luminescent agent is one that naturally emits or is altered to emit light. In particular embodiments, the luminescent agent is a fluorescent agent. The fluorescent agent may include adenosine or an analog thereof such as adenosine monophosphate (AMP) or analog thereof, or adenosine diphosphate (ADP) or an analog thereof. For example, any fluorescent dye that is conjugated to ATP, ADP or AMP can be used to probe interactions with AMPK as understood by one of ordinary skill in the art. Dyes compatible with this technology include Alexafluor® dyes such as Alexa 350, 405, 430, 488, 514, 532, 546, 555, 568, 594, 633, 635, 647, 660, 680, 700, 750, and 790; and Bodipy® (boron-dipyrromethene) dyes such as Bodipy FL, R6G, TMR, 581/591, TR, 630/650, and 650/665. In particular embodiments, the adenosine analog is methylanthraniloyl ADP or 2′-(or-3′)-O—(N-methylanthraniloyl)adenosine 5′-diphosphate (MANT-ADP), 2′-(or-3′)-O-(trinitrophenyl)adenosine 5′-diphosphate (TNP-ADP), Alexa Fluor®-ADP, or a combination thereof. The sample containing AMPK and the luminescent agent may include these reagents in a low concentration. For example, the fluorescent probe to protein ratio can be approximately 1:1000 (more protein than dye) to facilitate prolonged binding of the probe. Thus, with 1 micromolar AMPK protein, 1 nanomolar dye concentration may be utilized.

The sample including AMPK (as described above) and a luminescent agent is then contacted with a compound of interest. According to the present invention, the compounds of interest may be obtained from a commercial or proprietary library of compounds with known structural similarities to kinase inhibitors, have been shown to bind kinases, and/or are suspected to bind kinases. The kinases may be serine-threonine kinases or AMP kinases. In particular embodiments, the library of compounds, and thus, compound of interest, may be a small molecule compound. That is, the compound or compound of interest may have a low molecular weight. In some cases, the molecular weight is less than 900 Daltons. Exemplary sources of commercially available kinase libraries include SelleckChem kinase inhibitor library (Houston, Tex. and Munich, Germany) and ChemBridge™—KINASet (San Diego, Calif.).

The methods further provide that the luminescence in the sample prior to contacting the sample with the compound of interest is compared to the luminescence in the sample after contacting the sample with the compound of interest. Luminescence may refer to chemiluminescence, electroluminescence, mechanoluminescence, photoluminescence, in particular fluorescence, radioluminescence or thermoluminescence. In some embodiments, luminescence is detected using fluorimetry, fluorescence binding, fluorescence polarization, fluorescence resonance energy transfer (FRET) or time-resolved fluorescence resonance energy transfer (TR-FRET). According to embodiments of the present invention, a decrease in luminescence after contacting the sample with the compound of interest indicates that the compound of interest binds to AMPK. Further, a decrease in luminescence after contacting the sample with the compound of interest indicates that the compound of interest is a modulator of AMPK. Competitive binding of the compound of interest with the bound luminescent agent reverses an increase in luminescence indicating that the compound of interest is a modulator of AMPK. In some embodiments, sufficient levels of decreased fluorescence deemed as active compounds can be defined as at least 50% relative to the positive control. For example, if the starting fluorescence of the negative control is 200,000 relative fluorescent units (RFU), and 50,000 RFU for the positive control, a compound may be selected for follow up studies in methods of the present invention if they cross the 50% threshold at 125,000 RFU. However, in some embodiments, if the fluorescent-ADP analog is environmentally sensitive, then the fluorescent signal could either increase or decrease upon displacement indicating a small molecule binding event.

As used herein, “modulate” or “modulation” refers to enhancement (e.g., an increase) or inhibition (e.g., a reduction) in the activity of interest. Thus, an activator of AMPK may increase the activity associated with AMPK and an inhibitor may reduce the activity associated with AMPK.

Further confirmation of the ability of the compound identified by the methods described above can be achieved by subjecting the compound associated with the decrease in luminescence (or increase depending upon the environmental sensitivity of the luminescent agent employed) to a cell-based assay, an in vitro kinase assay, an in vitro phosphatase assay, or a combination thereof to test specific effects on AMPK. In some embodiments, a cell-based assay may be used to test the efficacy in modulating AMPK activity and may include: 1) increased/decreased phospho-AMPK, or 2) increased phospho-Acetyl-CoA (ACC) carboxylase. In such assays, HEK-293 cells may be treated with various concentrations of the test compound ranging from 1 micromolar to 20 nanomolar. Cells may be treated for 24 hours and the cell lysate harvested. Western blots are performed to establish an increase in phospho-AMPK for AMPK activators, and decrease for AMPK inhibitors. Additionally or alternatively, western blots are performed to establish an increase in phospho-ACC for AMPK activators, and decrease for AMPK inhibitors.

In addition to the methods described above, the present invention also provides assays for identifying a compound that modulates AMPK for the manufacture of a diagnostic or therapeutic agent. The assays comprise, consist essentially of, or consist of screening a compound of interest for its effect to displace a fluorescent AMPK ligand bound to AMPK. Displacement results in a decrease in luminescence and indicates that the compound of interest is a modulator of AMPK. Competitive displacement of bound fluorescent AMPK ligand by the compound of interest reverses an increase in luminescence indicating that the compound of interest is a modulator of AMPK. In some instances, there may be an increase in luminescence depending upon the environmental sensitivity of the luminescent agent employed. The assay may further include conducting a cell-based assay, an in vitro kinase assay, an in vitro phosphatase assay, or a combination thereof to test specific effects on AMPK as discussed above.

In particular embodiments, the assays are high-throughput assays as known to those skilled in the art. Briefly, the reagents may be placed in microplates including a grid of small wells typically in multiples of 96. Alternatively, the microplates may be replaced by drops of fluid separated by oil. As with the microplating technique, the microfluidic technique can be used in fluorescent measurements with adaptation. Utilizing either technique, the samples may be prepared, mixed, incubated, analyzed and or detected by automation allowing the rapid identification of compounds of interest.

The assay may be a fluorimetric assay, a labeled binding assay, a fluorescence polarization assay, a fluorescence resonance energy transfer (FRET) assay or a time-resolved fluorescence resonance energy transfer (TR-FRET) assay.

Where AMPK has been implicated in cellular and whole body metabolism, AMPK has been further implicated as a therapeutic target for illnesses characterized by abnormal energy regulation or metabolic disease, including diabetes and cancer. Compounds that target AMP kinase directly normalize glycemia and lipid profiles by at least increasing glucose uptake independently of insulin, increasing lipid oxidation and/or decreasing glucose and lipid production and/or restoring energy balance. See Poxel, Lyon, France.

As used herein, a “metabolic disease” or “metabolic disorder” (wherein disease and disorder can be used interchangeably) refers to a condition caused by an abnormal metabolic process. Common metabolic disorders include, but are not limited to, diabetes, insulin resistance, obesity, dyslipidemia, lypolipedemia, hyperthyroidism, hypothyroidism, galactosemia and phenylketonuria. “Diabetes” can refer to a disease diagnosed as diabetes according to the diagnostic standard, for example, of WHO (World Health Organization), Japan Diabetes Society, American Diabetes Association or European Association for the Study of Diabetes and includes Type 1 diabetes, Type 2 diabetes, gestational or pregnancy diabetes, and the like. Type 2 diabetes can be characterized by its resistance to the action of insulin, i.e., “insulin resistance.” “Insulin resistance” can mean a disease diagnosed as insulin resistance, based on the insulin resistance index (fasting blood sugar (mg/dL)×fasting insulin (microU/mL)÷405) or on the results obtained by examination by glucose clamp method or the like and includes syndrome X additionally. In addition to Type 2 diabetes, diseases with “insulin resistance” include, for example, fatty liver, particularly NAFLD (non-alcoholic fatty liver disease), NASH (non-alcoholic steatohepatitis), coronary heart diseases (CHDs), arteriosclerotic diseases, hyperglycemia, lipodosis, impaired glucose tolerance, hypertension, hyperlipemia, diabetes complications, pregnancy diabetes, polycystic ovary syndrome and the like.

Examples of cancers, tumors, and neoplastic tissue include, but are not limited to, malignant disorders such as breast cancers, osteosarcomas; angiosarcomas; fibrosarcomas and other sarcomas; leukemias; lymphomas; sinus tumors; ovarian, uretal, bladder, prostate and other genitourinary cancers; colon, esophageal and stomach cancers and other gastrointestinal cancers; lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas.

Thus, embodiments of the present invention further provide in vitro methods and assays that identify a compound that modulates AMPK for the manufacture of a diagnostic or therapeutic agent for illnesses characterized by abnormal energy regulation or metabolic disease, including diabetes and cancer as described above. Moreover, the compounds identified herein can be used to treat obesity, metabolic disease, including diabetes, cancer and other disorders described above.

The subjects to be treated according to the present invention include any subject in whom prevention and/or treatment of obesity, metabolic disease, including diabetes and cancer is needed or desired, as well as any subject prone to such a disorder(s). In some embodiments, the subject is a human; however, a subject of this invention can include an animal subject, particularly mammalian subjects such as canines, felines, bovines, caprines, equines, ovines, porcines, rodents (e.g. rats and mice), lagomorphs, primates (including non-human primates), etc., including domesticated animals, companion animals and wild animals for veterinary medicine or treatment or pharmaceutical drug development purposes.

The subjects relevant to this invention may be male or female and may be any species and of any race or ethnicity, including, but not limited to, Caucasian, African-American, African, Asian, Hispanic, Indian, etc., and combined backgrounds. The subjects may be of any age, including newborn, neonate, infant, child, adolescent, adult, and geriatric.

The compounds described herein may be formulated as pharmaceutical compositions including a pharmaceutically acceptable carrier. The particular choice of carrier and formulation will depend upon the particular route of administration for which the composition is intended.

The compositions of the present invention may be suitable for parenteral, oral, inhalation spray, topical, rectal, nasal, buccal, vaginal or implanted reservoir administration, etc. The term “parenteral” as used herein includes subcutaneous, intradermal, intravenous, intramuscular, intra-articular, intra-synovial, intrasternal, intrathecal, intrahepatic, intralesional and intracranial injection or infusion techniques.

The carriers and additives used for such pharmaceutical compositions can take a variety of forms depending on the anticipated mode of administration. Thus, compositions for oral administration may be, for example, solid preparations such as tablets, sugar-coated tablets, hard capsules, soft capsules, granules, powders and the like, with suitable carriers and additives being starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like. Typical preparations for parenteral administration comprise the active ingredient with a carrier such as sterile water or parenterally acceptable oil including polyethylene glycol, polyvinyl pyrrolidone, lecithin, arachis oil or sesame oil, with other additives for aiding solubility or preservation may also be included. In the case of a solution, it can be lyophilized to a powder and then reconstituted immediately prior to use. For dispersions and suspensions, appropriate carriers and additives include aqueous gums, celluloses, silicates or oils.

The pharmaceutical compositions according to embodiments of the present invention include those suitable for oral, rectal, topical, inhalation (e.g., via an aerosol) buccal (e.g., sub-lingual), vaginal, topical (i.e., both skin and mucosal surfaces, including airway surfaces), transdermal administration and parenteral (e.g., subcutaneous, intramuscular, intradermal, intraarticular, intrapleural, intraperitoneal, intrathecal, intracerebral, intracranially, intraarterial, or intravenous), although the most suitable route in any given case will depend on the nature and severity of the condition being treated and on the nature of the particular active agent which is being used.

Compositions for injection will include the active ingredient together with suitable carriers including propylene glycol-alcohol-water, isotonic water, sterile water for injection (USP), emulPhor™-alcohol-water, cremophor-EL™ or other suitable carriers known to those skilled in the art. These carriers may be used alone or in combination with other conventional solubilizing agents such as ethanol, propylene glycol, or other agents known to those skilled in the art.

Where the compounds described herein are to be applied in the form of solutions or injections, the compounds may be used by dissolving or suspending in any conventional diluent. The diluents may include, for example, physiological saline, Ringer's solution, an aqueous glucose solution, an aqueous dextrose solution, an alcohol, a fatty acid ester, glycerol, a glycol, an oil derived from plant or animal sources, a paraffin and the like. These preparations may be prepared according to any conventional method known to those skilled in the art.

Compositions for nasal administration may be formulated as aerosols, drops, powders and gels. Aerosol formulations typically comprise a solution or fine suspension of the active ingredient in a physiologically acceptable aqueous or non-aqueous solvent. Such formulations are typically presented in single or multidose quantities in a sterile form in a sealed container. The sealed container can be a cartridge or refill for use with an atomizing device. Alternatively, the sealed container may be a unitary dispensing device such as a single use nasal inhaler, pump atomizer or an aerosol dispenser fitted with a metering valve set to deliver a therapeutically effective amount, which is intended for disposal once the contents have been completely used. When the dosage form comprises an aerosol dispenser, it will contain a propellant such as a compressed gas, air as an example, or an organic propellant including a fluorochlorohydrocarbon or fluorohydrocarbon.

Compositions suitable for buccal or sublingual administration include tablets, lozenges and pastilles, wherein the active ingredient is formulated with a carrier such as sugar and acacia, tragacanth or gelatin and glycerin.

Pharmaceutically acceptable salts of the compounds described herein include a salt form of the compounds of the present invention in order to permit their use or formulation as pharmaceuticals and which retains the biological effectiveness of the free acids and bases of the specified compound and that is not biologically or otherwise undesirable. Examples of such salts are described in Handbook of Pharmaceutical Salts: Properties, Selection, and Use, Wermuth, C. G. and Stahl, P. H. (eds.), Wiley-Verlag Helvetica Acta, Zürich, 2002 [ISBN 3-906390-26-8]. Examples of such salts include alkali metal salts and addition salts of free acids and bases. Examples of pharmaceutically acceptable salts, without limitation, include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, xylenesulfonates, phenylacetates, phenylpropionates, phenylbutyrates, citrates, lactates, γ-hydroxybutyrates, glycollates, tartrates, methanesulfonates, ethane sulfonates, propanesulfonates, toluenesulfonates, naphthalene-1-sulfonates, naphthalene-2-sulfonates, and mandelates.

Embodiments of the present invention also provide kits including the elements necessary to carry out the processes described above. Such a kit may comprise a carrier being compartmentalized to receive in close confinement therein one or more containers, such as tubes or vials. One or more of the containers may contain a compound described herein. One or more containers may contain one or more enzymes or reagents to be utilized in desired reactions. These enzymes may be present by themselves or in admixtures, in lyophilized form or in appropriate buffers. The kit may contain all of the additional elements necessary to carry out techniques of the invention, such as buffers, control plasmid, oligonucleotides, extraction reagents, fixation agents, permeability agents, enzymes, pipettes, plates, nucleic acids, gel materials, transfer materials, autoradiography supplies, instructions and the like.

Embodiments of the present invention further provide a compound having the following structure and pharmaceutically acceptable salts thereof, a pharmaceutical composition of the same comprising a pharmaceutically acceptable carrier and kits further including:

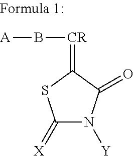

wherein:

A is a moiety selected from the group consisting of: aryl and benzyl; wherein each of said aryl or benzyl groups can optionally have from 0 to 3 substituents selected from the group consisting of: C₁-C₆ alkyl, C₃-C₇ cycloalkyl, benzyl, aryl, —(CO₂₋alkyl), alkoxy, aryloxy, amino, alkylamino, dialkylamino, amido, nitro and halogen;

B is a heterocyclic moiety selected from the group consisting of: substituted or unsubstituted furan, thiophene, azole, imidazole, pyrazole, oxazole, thiazole and isoxazole;

R is selected from the group consisting of: H, C₁-C₆ alkyl, C₃-C₇ cycloalkyl, benzyl and aryl wherein said benzyl and aryl substituents can optionally be unsubstituted or substituted with one or more groups selected from halogen; alkyl and alkoxy;

X═O or S;

Y is selected from the group consisting of H, —(CH₂)ₙ—CO₂H, —(CH₂)ₙ—SO₃H, ArCO₂H and ArSO₃H, wherein each CH₂ group can optionally be substituted with one or two alkyl groups and each aryl group can optionally be substituted with one or more groups selected from: halogen; alkyl and alkoxy; and

n=1-6; wherein the stereochemistry of the double bond between the thiazolidenyl moiety and the carbon bearing R and B can be either E or Z.

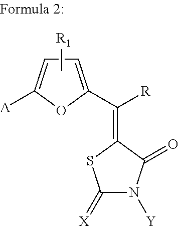

wherein A, R, X, and Y are defined as in Formula 1 and R₁ is selected from the group consisting of: H, C₁-C₆ alkyl, C₃-C₇ cycloalkyl, benzyl, aryl, alkoxy, aryloxy and halogen.

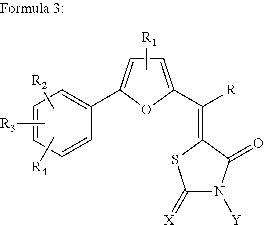

wherein R₁, R, X, and Y are defined as in Formula 2 and groups R₂₋₄ are independently selected from the group consisting of: H, C₁-C₆ alkyl, C₃-C₇ cycloallcyl, benzyl, aryl, —(CO₂₋alkyl), alkoxy, aryloxy and halogen.

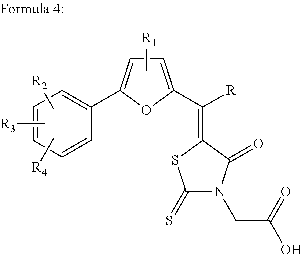

wherein R₁-R₄ are defined as in Formula 3.

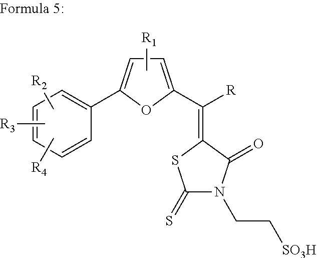

wherein R₁-R₄ are defined as in Formula 3.

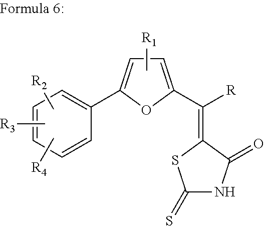

wherein R₁-R₄ are defined as in Formula 3.

In some embodiments, the compound is not one of the following compounds, that is, the formulas above include a proviso wherein the compounds of the formulas recited above do not include one or more of the following compounds or any other known compounds:

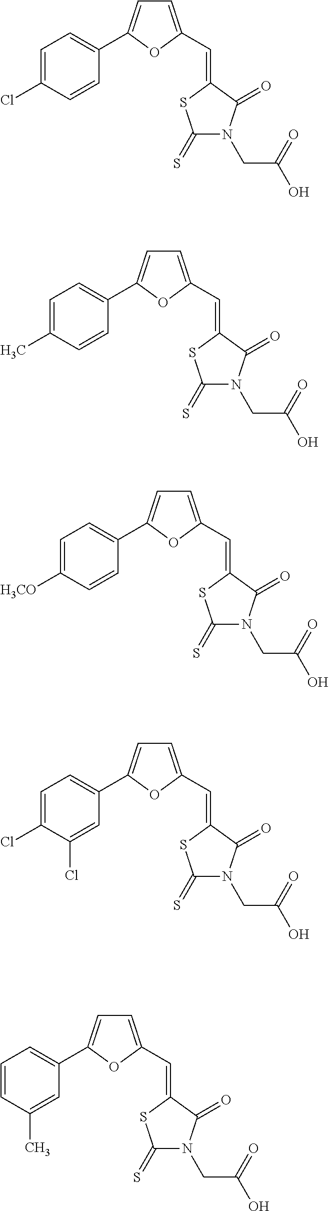 [SMILES: O=C(O)CN1C(=O)/C(=C/c2ccc(-c3ccc(Cl)cc3)o2)SC1=S ; Cc1ccc(-c2ccc(/C=C3\SC(=S)N(CC(=O)O)C3=O)o2)cc1 ; COc1ccc(-c2ccc(/C=C3\SC(=S)N(CC(=O)O)C3=O)o2)cc1 ; O=C(O)CN1C(=O)/C(=C/c2ccc(-c3ccc(Cl)c(Cl)c3)o2)SC1=S ; Cc1cccc(-c2ccc(/C=C3\SC(=S)N(CC(=O)O)C3=O)o2)c1]

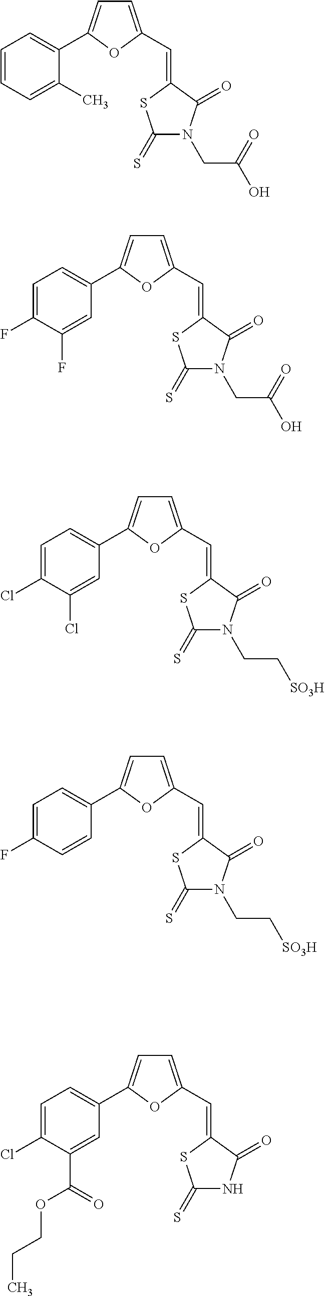 [SMILES: Cc1ccccc1-c1ccc(/C=C2\SC(=S)N(CC(=O)O)C2=O)o1 ; O=C(O)CN1C(=O)/C(=C/c2ccc(-c3ccc(F)c(F)c3)o2)SC1=S ; O=C1/C(=C/c2ccc(-c3ccc(Cl)c(Cl)c3)o2)SC(=S)N1CCS(=O)(=O)O ; O=C1/C(=C/c2ccc(-c3ccc(F)cc3)o2)SC(=S)N1CCS(=O)(=O)O ; CCCOC(=O)c1cc(-c2ccc(/C=C3\SC(=S)NC3=O)o2)ccc1Cl]

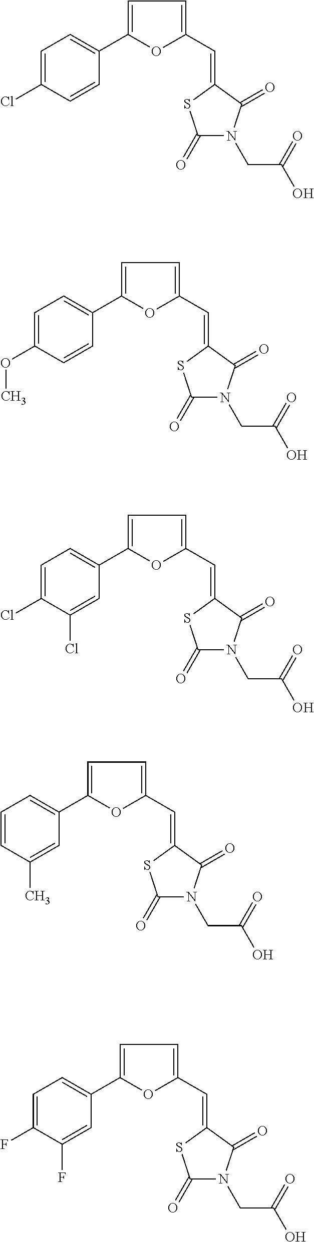 [SMILES: O=C(O)CN1C(=O)S/C(=C\c2ccc(-c3ccc(Cl)cc3)o2)C1=O ; COc1ccc(-c2ccc(/C=C3\SC(=O)N(CC(=O)O)C3=O)o2)cc1 ; O=C(O)CN1C(=O)S/C(=C\c2ccc(-c3ccc(Cl)c(Cl)c3)o2)C1=O ; Cc1cccc(-c2ccc(/C=C3\SC(=O)N(CC(=O)O)C3=O)o2)c1 ; O=C(O)CN1C(=O)S/C(=C\c2ccc(-c3ccc(F)c(F)c3)o2)C1=O]

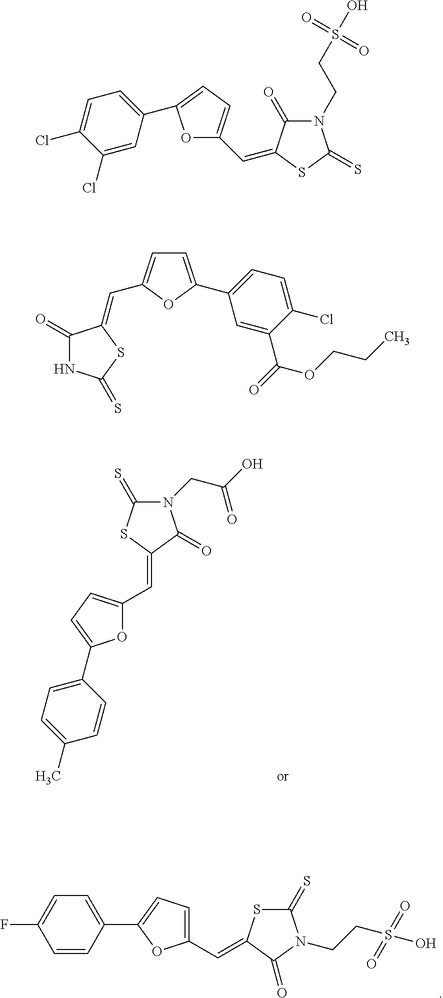 [SMILES: O=C1/C(=C\c2ccc(-c3ccc(Cl)c(Cl)c3)o2)SC(=S)N1CCS(=O)(=O)O ; CCCOC(=O)c1cc(-c2ccc(/C=C3\SC(=S)NC3=O)o2)ccc1Cl ; Cc1ccc(-c2ccc(/C=C3\SC(=S)N(CC(=O)O)C3=O)o2)cc1 ; O=C1/C(=C/c2ccc(-c3ccc(F)cc3)o2)SC(=S)N1CCS(=O)(=O)O]

The general procedure for implementing the methods and assays of the present invention can be readily understood and appreciated by one skilled in the art. Some aspects of the present invention are described in more detail in the following non-limiting Examples. These are not intended to restrict the present invention, and may be modified within the range not deviating from the scope of this invention.

### EXAMPLES

A. Initial Testing

Many AMPK modulators produce AMPK-independent effects. To identify drugs that modulate AMPK activity independent of the canonical ATP-binding pocket found throughout the kinome, a fluorescence-based high-throughput screening assay biased toward the identification of molecules that bind the regulatory region of AMPK was designed. Automated pintools were used to rapidly transfer small molecules to a low volume assay mixture on 384-well plates. Prior to assay validation, a full assay optimization to maximize the signal-to-background and reduce variability for enhanced detection of small molecules displacing MANT-ADP was completed. After validation, 13,120 molecules were screened and 3 positive hits identified that dose-dependently inhibited the protein-bound signal of MANT-ADP in the presence of both full-length AMPK and the truncated regulatory fragment of AMPK, which is missing the kinase active site. The average Z′-factor for the screen was 0.55 and the compound confirmation rate was 60%. Thus, a luminescence-based assay may be paired with in vitro kinase assays and cell-based assays to identify molecules that selectively regulate AMPK with fewer off-target effects on other kinases.

### Example 1

### Fluorescence-Based Assay to Identify Molecules Binding Full-Length AMPK and AMPK Regulatory Fragment

### Materials

STK740822 (Vitas-M Laboratory, Ltd.), STL035166 (Vitas-M Laboratory, Ltd.), and Z64358107 (Enamine, Ltd.) were re-purchased in powder form and then dissolved in DMSO for dose response studies. ADP, lysozyme, and 1% EDTA-free protease inhibitor cocktail were purchased from Sigma-Aldrich. MANT-ADP was purchased from Life Technologies. Cobalt-based immobilized metal affinity chromatography (IMAC) resin was purchased from Clontech Laboratories, Inc.

### Protein Purification

Tricistronic vectors encoding full-length AMPK (rat His-α₁, β₁, γ₁) and the AMPK regulatory fragment (rat His-α₁, 396-548; human β₂, 187-272; rat γ₁) were gifts from Dr. Uwe Schlattner and Dr. Steve Gamblin, respectively. Vectors were transformed into Rosetta cells and individual colonies were then incubated at 37° C. in overnight starter cultures containing antibiotics as would be understood by those of ordinary skill in the art. Overnight cultures were amplified in auto-inducible media and shaken for two days at room temperature. Induced cultures were pelleted, washed twice in 0.9% NaCl, and then sonicated (40% intensity, 30 seconds, 3 times with 2 minute intervals) at 4° C. in lysis buffer containing 50 mM Tris-HCl (pH 8), 100 mM NaCl, 0.75 mg/mL lysozyme, 0.1% Triton X100, and 1% EDTA-free protease inhibitor cocktail. Lysates were centrifuged and supernatants were batch-bound onto cobalt-based IMAC resin. Resin was washed three times in buffer containing 0.01% Triton X100 (first wash only), 50 mM Tris-HCl (pH 8), 100 mM NaCl, and 2 mM imidazole. Washed resin was loaded into a column prior to elution with a solution of 50 mM Tris-HCl (pH 8), 100 mM NaCl, and 500 mM imidazole. Eluates were concentrated on either 30 kDa (for the regulatory fragment) or 50 kDa (for full-length AMPK) size exclusion centrifugal filters at 4° C. Concentrates were resuspended and concentrated twice more in 50 mM Tris-HCl (pH 8) prior to storage at −80° C.

### Assay Assembly

Except where otherwise noted, assays were performed with the following conditions: 0.5 μM full-length AMPK (130 kDa) or 0.5 μM regulatory fragment (66.7 kDa), 0.1 μM MANT-ADP, 10 mM Tris-HCl (pH 8), 0.45% DMSO (vehicle and negative control), and 5 μM ADP (positive control). Automated assays were assembled in two steps. First, a NanoQuot (BioTek) was used to dispense 11 μL of master mix (protein, MANT-ADP, and Tris-HCl) per well in black, low volume 384-well plates (Corning-3676). Next, a Biomek NX workstation (Beckman-Coulter, Brea, Calif.) equipped with pintools (VP Scientific, San Diego Calif.) was used to transfer 50 nL of controls (columns 1, 2, 23, and 24) and small molecules (4.5 μM final). Controls and library molecules were transferred from Axygen 384-well rigid PCR plates (Cat no. 321-67-051) at 221× concentration for single-concentration screening. Plates were shaken for 10 minutes prior to reading.

### Fluorescence Measurements

Fluorescence emission spectra of MANT-ADP were collected using a PerkinElmer EnSpire plate reader with an excitation wavelength of 360 nm. Background fluorescence from protein was subtracted from the raw data prior to plotting MANT-ADP's fluorescence in the presence of protein. Fluorescence detection for assay development and screening was performed on either a BMG Pherastar Plus (360/10 excitation and 460/10 emission filters) or a PerkinElmer EnVision (355/40 excitation and 460/25 emission filters), depending on equipment availability. Relative fluorescence units (RFUs) were recorded at room temperature.

### Calculation of Z′

Z′ was calculated using the formula: Z′=1-((3(σ_{vehicle}+σ_{ADP}))/(μ_{vehicle}−μ_{ADP})).

### Small Molecule Library

The Center for Integrative Chemical Biology and Drug Discovery (CICBDD) assembled a commercially-available small molecule library from multiple sources comprised of molecules that have structural similarities to known kinase inhibitors and/or have been shown to bind kinases in silico (Hutti J E, Porter M A, Cheely A W, et al. Development of a high-throughput assay for identifying inhibitors of TBK1 and IKKepsilon. PLoS One 2012; 7(7):e41494; Peterson E J, Janzen W P, Kireev D, Singleton S F. High-throughput screening for RecA inhibitors using a transcreener adenosine 5′-O-diphosphate assay. Assay Drug Dev Technol 2012; 10(3):260-8). Library molecules were stored at 1 mM concentration in DMSO. All of the molecules comply with Lipinski's rules (.Lipinski C A; Lombardo F, Dominy B W, Feeney P J. Experimental and computational approaches to estimate solubility and permeability in drug discovery and development settings. Advanced Drug Delivery Reviews 1997; 23(1-3):3-25).

### Screening Data Analysis

ScreenAble (ScreenAble Solutions, Chapel Hill, N.C.) high-throughput screening software was utilized for merging screening data and chemical structure, for statistical design of experiments, and hit selection. GraphPad Prism was used for non-linear regression analysis of dose response data.

### Results

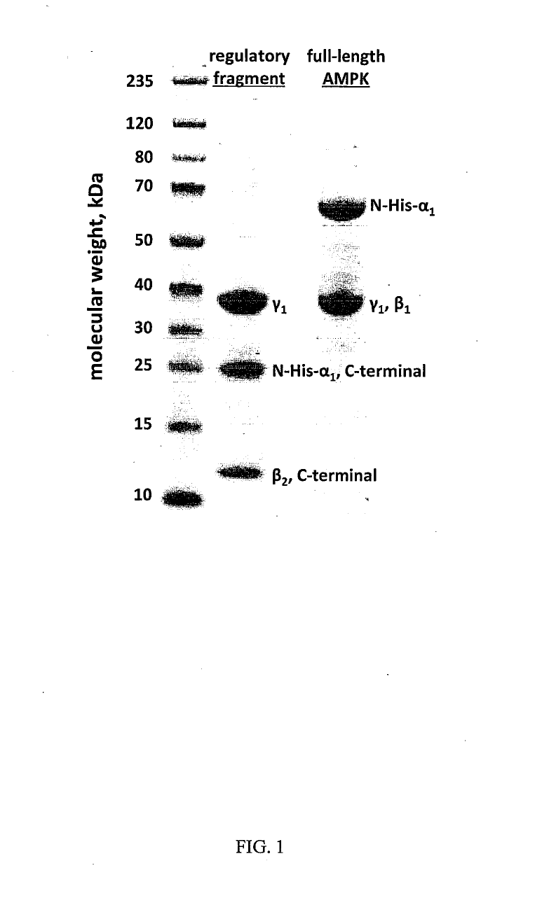

The overall goal of this study was to identify novel small molecules that preferentially bind the regulatory region of AMPK. To achieve this goal, a fluorescence-based assay using purified His-tagged AMPK trimer and a fluorescent analog of AMPK's primary regulatory nucleotide, ADP was designed (FIG. 1). Optimizing lysis conditions and switching from isopropyl beta-D-thiogalactopyranoside (IPTG)-inducible media to auto-inducible media increased the yield of purified protein from 3 mgs to 20 mgs per liter of culture, greater than previously published yields for AMPK heterotrimers (Neumann D, Woods A, Carling D, Wallimann T, Schlattner U. Mammalian AMP-activated protein kinase: functional, heterotrimeric complexes by co-expression of subunits in Escherichia coli. Protein Expr Purif 2003; 30(2):230-7; Rajamohan F, Harris M S, Frisbie R K, et al. Escherichia coli expression, purification and characterization of functional full-length recombinant alpha2beta2gamma3 heterotrimeric complex of human AMP-activated protein kinase. Protein Expr Purif 2010; 73(2):189-97; Riek U, Scholz R, Konarev P, et al. Structural properties of AMP-activated protein kinase: dimerization, molecular shape, and changes upon ligand binding. J Biol Chem 2008; 283(26):18331-43). This improved AMPK expression method supplied ample protein for designing and optimizing assays, screening libraries, and confirming hits in secondary assays.

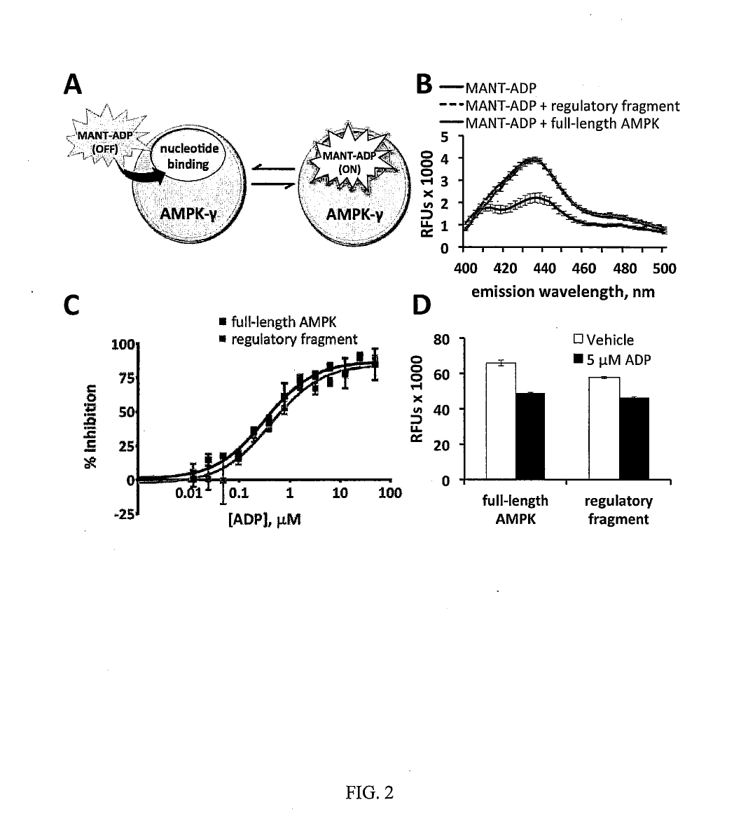

Both MANT-labeled and trinitrophenylated (TNP) nucleotide analogs are environment-sensitive probes whose fluorescence increases upon binding to nucleotide-binding sites on protein (FIG. 2A) (Xiao B, Heath R, Saiu P, et al. Structural basis for AMP binding to mammalian AMP-activated protein kinase. Nature 2007; 449(7161):496-500; Saiu P. Structural and functional studies on nucleotide binding to AMP-activated protein kinase. London: University College London; 2010; accessed March 2013; http://discovery.ucl.ac.uk/645676/1/645676.pdf.; Guarnieri M T, Blagg B S, Zhao R. A high-throughput TNP-ATP displacement assay for screening inhibitors of ATP-binding in bacterial histidine kinases. Assay Drug Dev Technol 2011; 9(2):174-83). Fluorescence emission spectra were recorded for MANT-ADP and TNP-ADP (0.1 μM each) in the presence and absence of protein (FIG. 2B). Only MANT-ADP fluorescence increased upon addition of full-length AMPK and the regulatory fragment (FIG. 2B). MANT-ADP was therefore selected for detection of small molecules binding to the regulatory fragment of AMPK. The general assay principle is to pre-load AMPK with MANT-ADP, causing an increase in MANT-ADP fluorescence. If a small molecule can displace MANT-ADP from the binding site, MANT-ADP will be ejected and will have a substantial decrease in fluorescence, thereby indicating a positive result for the test compound. Using excess AMPK (0.5 μM protein with 0.1 μM MANT-ADP) helped maximize the number of MANT-ADP molecules bound to protein and thus helped maximize the protein-bound fluorescent signal in the absence of the competitive positive control ADP.

ADP, which competitively binds to Site 1 and Site 3 on AMPK-γ, inhibited the increase in MANT-ADP fluorescence with IC50s of 0.4 μM and 0.3 μM for the regulatory fragment and full-length AMPK, respectively (FIG. 2C). For the ADP dose responses, replicates containing MANT-ADP with no protein were used as positive controls for 100% inhibition of MANT-ADP's protein-bound fluorescent signal. Although the signal-to-background ratio was less than 2-fold (FIGS. 2B, 2D), the assay's Z′-factor was greater than 0.6 (FIG. 2D), indicating that the assay was robust enough for high throughput screening. At an emission wavelength of 460 nm, full-length AMPK consistently provided a slightly larger assay window, usually resulting in higher Z′-factors (FIG. 2D). The small molecule library, therefore, was screened against full-length AMPK. Positive hits were confirmed against the regulatory fragment in subsequent secondary screens. Aside from a small difference in assay window, truncation of AMPK-α₁ and AMPK-β₂ did not appear to significantly disrupt interactions among AMPK-γ₁, MANT-ADP, and ADP.

Prior to screening, assay conditions were optimized by testing high and low concentrations of several reagents in a design of experiments study using ScreenAble software (ScreenAble Solutions, Chapel Hill, N.C.). Previous studies have shown that affinities of adenine nucleotides for AMPK decrease with increasing ionic strength (Xiao B,

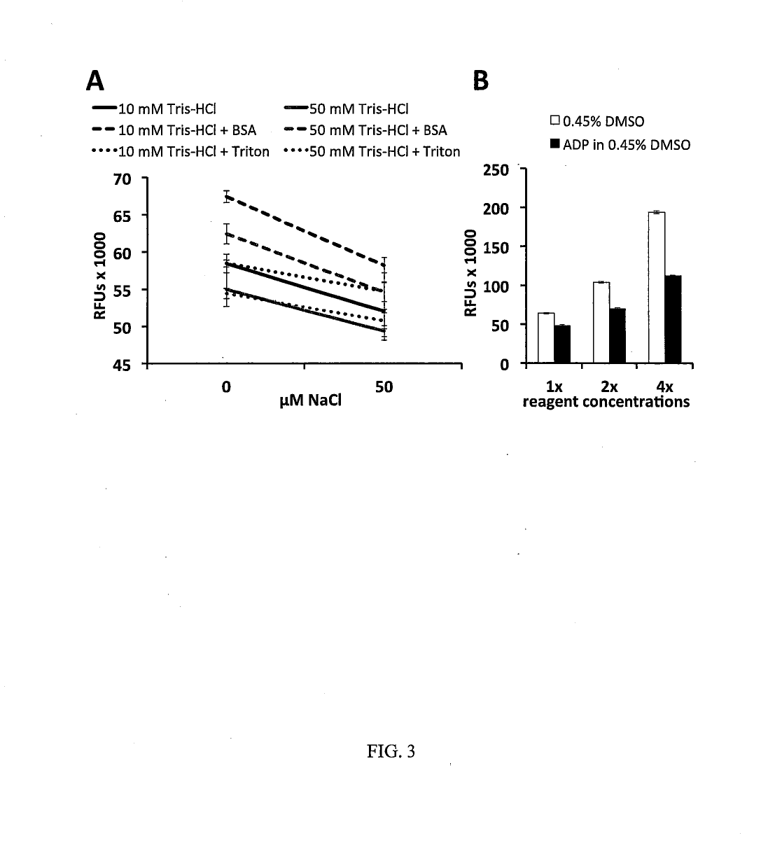

Heath R, Saiu P, et al. Structural basis for AMP binding to mammalian AMP-activated protein kinase. Nature 2007; 449(7161):496-500; Saiu P. Structural and functional studies on nucleotide binding to AMP-activated protein kinase. London: University College London; 2010; accessed March 2013; http://discovery.ucl.ac.uk/645676/1/645676.pdf). In agreement with published data, the greatest MANT-ADP fluorescence was observed with a low concentration of Tris-HCl (pH 8) and 0 μM NaCl (FIG. 3A). Triton, which is often used to prevent adsorption of target proteins onto plastic, had no effect on MANT-ADP fluorescence in the presence of 0 μM NaCl (FIG. 3A) (Simpson R J. Stabilization of proteins for storage. Cold Spring Harb Protoc 2010; 2010(5):pdb top79). The sacrificial protein BSA did increase fluorescence (FIG. 3A), but this was due in part to interactions between BSA and MANT-ADP. In the absence of AMPK, MANT-ADP's fluorescence still increased upon addition of BSA, even after subtracting BSA's autofluorescence from the raw data (not shown). It is possible that MANT-ADP binds non-specifically to BSA, thus decreasing the pool of MANT-ADP molecules that can bind to AMPK and consequently decreasing the assay window between vehicle and ADP-treated control groups. Because BSA decreased the assay window and Z′-factor, we decided to exclude BSA from our optimized assay conditions. Optimized buffer conditions yielded a Z′-factor≧0.6 with an assay window that increased linearly with protein and MANT-ADP concentrations. Instead of increasing AMPK and MANT-ADP concentrations to maximize the assay window, we decided to optimize the assay with low reagent concentrations (0.5 μM AMPK and 0.1 μM MANT-ADP) to ensure sensitivity for small molecule binding, as micromolar concentrations of AMPK would severely limit the theoretical maximum inhibition due to the stoichiometry of enzyme to small molecule.

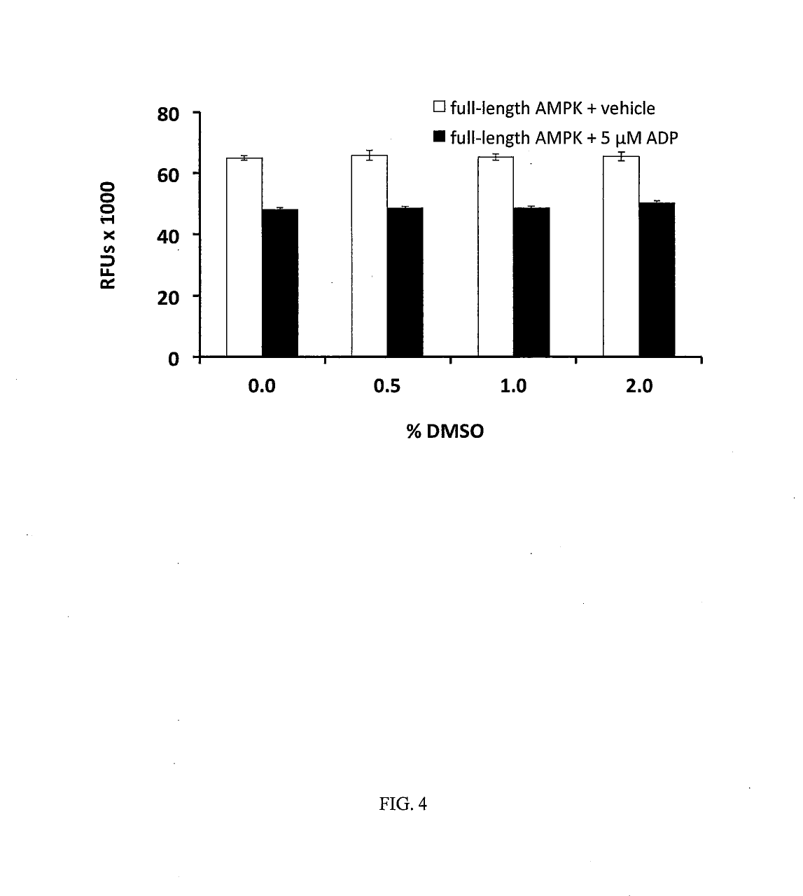

Since many small molecule libraries use DMSO as a solvent, the DMSO tolerance of the optimized assay was determined prior to screening. The assay yielded a robust Z′ across a range of DMSO concentrations, with no statistical difference observed among controls treated with 0-2% DMSO (FIG. 4). This DMSO tolerance is especially important for automated assay assembly, in which the final DMSO concentration may vary depending on the type of robotic dispenser used for transferring small molecules. The DMSO tolerance also allows flexibility in starting concentrations of library plate stocks.

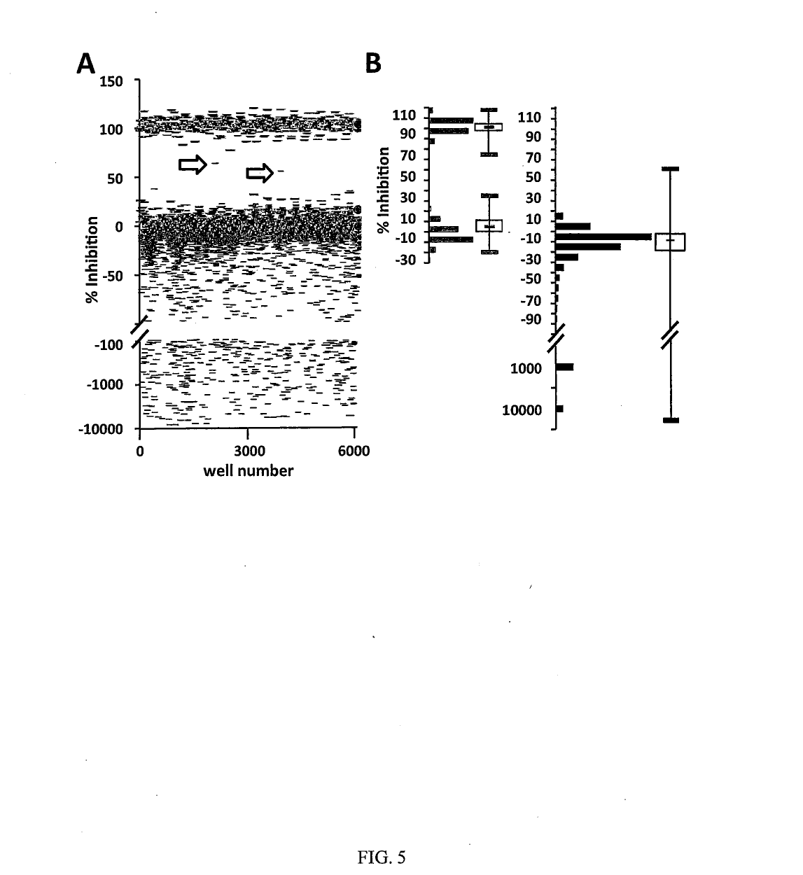

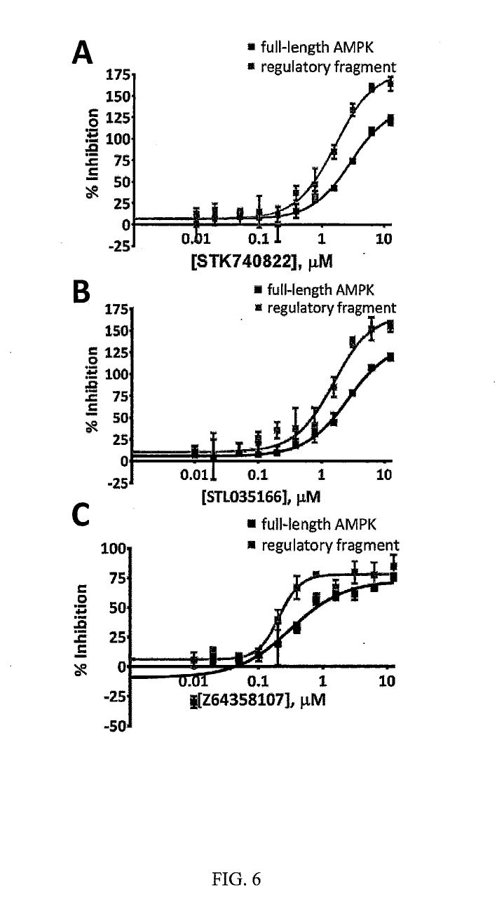

For library screening, a NanoQuot and a Biomek NX workstation were used to dispense the assay master mix and small molecules, respectively. To validate the automated dispensers, the plate-to-plate coefficient of variation (CV) was calculated for the control groups on 4 plates (<4% CV between plates; average Z′-factor=0.6) (data not shown). Fluorescence detection was performed on a BMG Pherastar Plus, which produced lower signals than the PerkinElmer EnVision used for other FIGS. (data not shown). The fold-difference between MANT-ADP's protein-bound and unbound signals, however, was similar for both detectors. After validating minimal plate-to-plate variation, 13,120 molecules from a small molecule library were screened over the course of several days. Five positive hits (0.04% primary hit rate) were identified, each of which inhibited MANT-ADP fluorescence by more than 50% (FIG. 5). Autofluorescent molecules increased the total fluorescent signal and are shown on the scatter plot as having large negative binding activity (FIG. 5A). Data for DMSO controls and library molecules had similar distributions, with the average compound binding activity close to 0% inhibition of MANT-ADP fluorescence (FIG. 5B). The large number of autofluorescent molecules, which decreased the efficiency of screening, may explain the low primary hit rate. It is possible that some of the autofluorescent molecules are false negatives that inhibit binding of MANT-ADP but are masked by their autofluorescence. After initial screening, positive hits were re-purchased in powder form, dissolved in DMSO and re-tested for inhibition of MANT-ADP fluorescence. Three of the 5 positive hits (60% confirmation rate) dose-dependently inhibited MANT-ADP fluorescence in the presence of full-length AMPK (FIG. 6, Table 2 shown below). These 3 hits also dose-dependently inhibited fluorescence in the presence of the regulatory fragment (FIG. 6, Table 2). Like the primary assay using full-length AMPK, the secondary assay was performed in buffer that had low ionic strength and was assembled in the same manner, with controls and positive hits added to a master mix of regulatory fragment and MANT-ADP.

TABLE 2
Hill slopes, IC₅₀ₛ, and maximum inhibition were calculated for each of the three
reproducible hits in the presence of full-length AMPK and the regulatory fragment (data in
parentheses).

| Positive | Name or catalog | | | |
|---|---|---|---|---|
| hit | number | Structure | Hill slope | IC50, μM |
| 1 | STK740822, Vitas- M Laboratory, Ltd. | 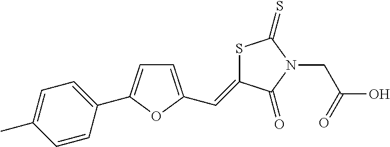 [SMILES: Cc1ccc(-c2ccc(/C=C3\SC(=S)N(CC(=O)O)C3=O)o2)cc1] | 1.4 (1.4) | 2.9 (1.6) |
| 2 | STL035166, Vitas- M Laboratory, Ltd. | 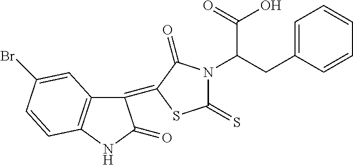 [SMILES: O=C1Nc2ccc(Br)cc2/C1=C1/SC(=S)N(C(Cc2ccccc2)C(=O)O)C1=O] | 1.3 (1.4) | 2.7 (1.5) |
| 3 | Z64358107, Enamine, Ltd. | 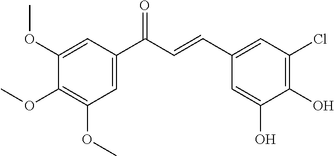 [SMILES: COc1cc(C(=O)/C=C/c2cc(O)c(O)c(Cl)c2)cc(OC)c1OC] | 1.1 (2.6) | 0.3 (0.2) |

Further studies resulted in 11 positive hits and confirmed the results seen with the three positive hits presented above.

### Conclusion

This screen has identified small molecules that are capable of inhibiting MANT-ADP's protein-bound fluorescent signal. Small molecules that dose-dependently inhibit MANT-ADP fluorescence may include AMPK inhibitors or activators.

### Example 2

Fluorescent Polarization Assay to Identify Molecules Binding AMPK Regulatory Fragment

Plate acceptability criteria is Z′>0.5 although we routinely achieve Z′>0.8 with the MANT-ADP assay and automated assembly routinely. Our day-to-day and plate-to-plate variations are typically minimal (CV<4%), indicating robust laboratory automation protocols. A two-stage screening approach was successfully employed, first screening against the AMPKγ regulatory subunit, followed by counterscreening the fully active AMPK trimer containing the kinase domain. Counterscreening against the active trimer provides assurance that any compounds with AMPK-regulatory domain binding activity will bind to functionally active protein but not target the kinase domain. Compounds found to bind the regulatory fragment from previous screening efforts all bound both to the regulatory fragment and full AMPK Trimer with similar affinities and Hill slope kinetics (Sinnett, S. E., Sexton, J. Z. & Brenman, J. E. A High Throughput Assay for Discovery of Small Molecules that Bind AMP-activated Protein Kinase (AMPK). Current Chemical Genomics and Translational Medicine Accepted (2013)). The main benefit of developing the FP assay will be using a red-shifted fluorophore with a much lower rate of autofluorescence for small molecules in compound libraries. Using an ADP-Alexa-647 conjugate will therefore increase the primary screening efficiency by 10-20% as the library molecule autofluorescence will be decreased.

### Screen ˜89,600 Small Molecule Compounds

Having optimized screening conditions and conducted successful pilot screens, we will screen an in-house, proprietary diversity library of 33,600 compounds and the commercially available Asinex library (56,000) against AMPKγ—a primary screen. Activities in each plate will be normalized to two rows of positive control wells (5 μM ADP+assay mix) and 2 rows of negative control wells (vehicle/DMSO alone) to ensure consistency in activity score across the screen. Z′ is calculated according to the formula: Z′=1-((3(σ_{vehicle}+σ_{ADP}))/(μ_{vehicle}−μ_{ADP})), and we will use a 0.5 Z-prime cutoff for plate acceptability. Hits will be selected based on a 50% activity threshold (approximately mean+6Xstdev). Dose-responsiveness for hits will be evaluated in 10-point/2-fold dilution format between 20 μM-40 nM. Compounds that show dose-responsiveness in a sigmoidal fashion will be repurchased or resynthesized and retested and confirmed for integrity by LC/MS as true positives.

Dose-responsive compounds with IC50s below 10 μM will be used for testing against the full-length AMPK trimer—a secondary screen. Compounds that are dose-responsive against both full length and the regulatory fragment will be prioritized for testing in cells in vitro below.

### Example 3

### Modulation of AMPK Activity In Vivo and In Vitro Detected Using Kinase and Phosphatase Protection Assays

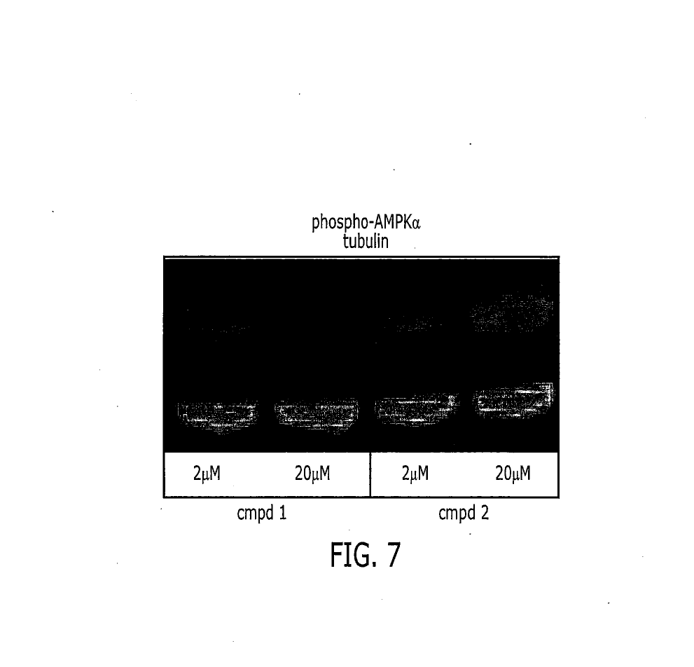

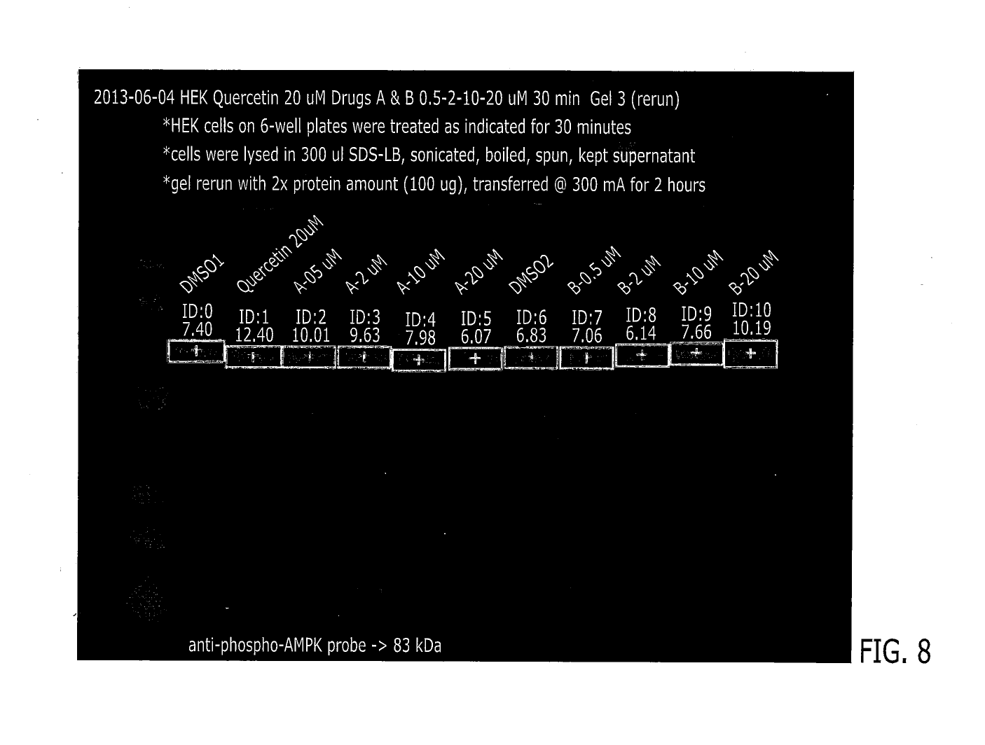

Approximately 50 compounds will be tested from those identified above using the methods and assays described herein on HEK cells in culture in 6 well plate format for their ability to enter cells and modulate AMPK, initially at 1 μM, 2 μM, 10 μM and 20 μM compound in 0.1% DMSO. HEK (human embryonic kidney) cells express functional AMPK protein capable of being super-activated by treatment with cobalt chloride, metformin, or oligomycin; conversely, AMPK activity can be diminished with high nutrient content media (Onyenwoke, R. U. et al. AMPK directly inhibits NDPK through a phosphoserine switch to maintain cellular homeostasis. Molecular biology of the cell 23, 381-389, doi:10.1091/mbc.E11-08-0699 (2012)). Compounds are tested on cells at basal states in culture in standard DMEM/glucose/10% FBS and also under AMPK-activating conditions as we have done before Onyenwoke, R. U. et al. AMPK directly inhibits NDPK through a phosphoserine switch to maintain cellular homeostasis. Molecular biology of the cell 23, 381-389, doi:10.1091/mbc.E11-08-0699 (2012)). Examples of this approach with compounds identified from our preliminary screening that have AMPK modulatory activity are shown in FIG. 7. One compound has inhibitory activity (STL035166), while one (STK740822) demonstrates activating activity. See also FIG. 8. Other well-known AMPK activating conditions including metformin addition and conditions that do not depend on added synthetic chemicals, including low glucose and/or serum deprivation are employed. By testing under multiple conditions, the likelihood of identifying agonists and antagonists of AMPK activity in vivo, and particularly those that are not dependent on a particular means of altering AMPK activity may increase.

### Measure Phospho-AMPKα and Direct Target, Phospho-ACC Phosphorylation Status

The phosphorylation status of AMPKα with a phospho-specific antibody (against the activation/T-loop threonine 172) (Cell Signaling) will be quantified. LiCor Odyssey fluorescent Western blot detection may be used as performed previously for AMPKα (Onyenwoke, R. U. et al. AMPK directly inhibits NDPK through a phosphoserine switch to maintain cellular homeostasis. Molecular biology of the cell 23, 381-389, doi:10.1091/mbc.E11-08-0699 (2012); Kazgan, N., Williams, T., Forsberg, L. J. & Brenman, J. E. Identification of a nuclear export signal in the catalytic subunit of AMP-activated protein kinase. Molecular biology of the cell 21, 3433-3442, doi:10.1091/mbc.E10-04-0347 (2010)). Phosphorylation of this particular residue (T172) is implicated in AMPK catalytic activity (Hawley, S. A. et al. Complexes between the LKB1 tumor suppressor, STRAD alpha/beta and MO25 alpha/beta are upstream kinases in the AMP-activated protein kinase cascade. Journal of biology 2, 28, doi:10.1186/1475-4924-2-28 (2003)) and can easily be monitored by Western blot (Onyenwoke, R. U. et al. AMPK directly inhibits NDPK through a phosphoserine switch to maintain cellular homeostasis. Molecular biology of the cell 23, 381-389, doi:10.1091/mbc.E11-08-0699 (2012); Kazgan, N., Williams, T., Forsberg, L. J. & Brenman, J. E. Identification of a nuclear export signal in the catalytic subunit of AMP-activated protein kinase. Molecular biology of the cell 21, 3433-3442, doi:10.1091/mbc.E10-04-0347 (2010)) (FIG. 7). (Phospho-AMPKα levels will be standardized both to tubulin and total AMPKα levels to ensure we are not simply detecting changes in total AMPKα protein levels.) Compound treatment will be performed for 1 hour (Onyenwoke, R. U. et al. AMPK directly inhibits NDPK through a phosphoserine switch to maintain cellular homeostasis. Molecular biology of the cell 23, 381-389, doi:10.1091/mbc.E11-08-0699 (2012), which is sufficient and maximal for known AMPK modulators and thus decreases the chances of seeing indirect downstream (e.g. transcriptional, toxicity) events caused by compounds. Compounds that do not show at least a 25% change (either increase or decrease) in phospho-AMPKα (standardized to tubulin and total AMPKα) as measured by fluorescent quantitative western blot will be triaged. In addition, phospho-ACC (Cell Signaling) will be measured by western blot. Cytoplasmic acetyl coenzyme A carboxylase (ACC) is the best known AMPK direct downstream target, and is a rate-limiting enzyme in the energy consuming process of fatty acid synthesis (Davies, S. P. et al. Purification of the AMP-activated protein kinase on ATP-gamma-sepharose and analysis of its subunit structure. European journal of biochemistry/FEBS 223, 351-357 (1994); Carling, D., Zammit, V. A. & Hardie, D. G. A common bicyclic protein kinase cascade inactivates the regulatory enzymes of fatty acid and cholesterol biosynthesis. FEBS Lett 223, 217-222 (1987)). Phospho-ACC will be standardized to both tubulin and total (non-phospho) ACC to ensure we are not measuring changes in total ACC levels. Compounds that modulate phospho-AMPKα and phospho-ACC status in the same direction (both increase or both decrease) will be prioritized over compounds that produce different affects on AMPKα versus ACC phosphorylation for in vitro kinase or phosphatase protection assays.

In Vitro Kinase Assays and Phosphatase Protection Assays for Compounds with Bioactivity to Gain Mechanism of Action Insight

For those small molecules that show AMPK modulatory activity in vivo, AMPK in vitro kinase and phosphatase protection assays will be performed. AMPK in vitro kinase assays will be performed using a well-documented synthetic peptide (“SAMS peptide”) substrate for AMPK activity and detailed protocol (Witters, L. A. & Kemp, B. E. Insulin activation of acetyl-CoA carboxylase accompanied by inhibition of the 5′-AMP-activated protein kinase. J Biol Chem 267, 2864-2867 (1992); Hamilton, S. R. et al. AMP-activated protein kinase kinase: detection with recombinant AMPK alpha1 subunit. Biochem Biophys Res Commun 293, 892-898, doi:10.1016/S0006-291X(02)00312-1 50006-291X(02)00312-1 [pii] (2002)). The SAMS synthetic peptide (SAMS peptide—HMRSAMSGLHLVKRR) has the endogenous Protein Kinase A (PKA) site mutated out but retains a single AMPK target serine residue (serine 79 in ACC) (Davies, S. P. et al. Purification of the AMP-activated protein kinase on ATP-gamma-sepharose and analysis of its subunit structure. European journal of biochemistry/FEBS 223, 351-357 (1994)). Synthetic C-terminal added positive charges (K/R) allow for simple filter-binding ATP-γ-P³²-based kinase assays (Carling, D., Clarke, P. R., Zammit, V. A. & Hardie, D. G. Purification and characterization of the AMP-activated protein kinase. Copurification of acetyl-CoA carboxylase kinase and 3-hydroxy-3-methylglutaryl-CoA reductase kinase activities. European journal of biochemistry/FEBS 186, 129-136 (1989)), as performed previously (Onyenwoke, R. U. et al. AMPK directly inhibits NDPK through a phosphoserine switch to maintain cellular homeostasis. Molecular biology of the cell 23, 381-389, doi:10.1091/mbc.E11-08-0699 (2012)). Purified AMPK complex will be incubated with small molecule compounds at the IC₅₀ concentrations for initial study. Purified AMPK complex/small molecule binders will then be used in standard AMPK kinase assays using radiolabeled γP³²-ATP/synthetic SAMS peptide incubation and subsequent P81 filter paper spotting and phosphoric acid wash protocols to measure P³² incorporation into the SAMS peptide (Witters, L. A. & Kemp, B. E. Insulin activation of acetyl-CoA carboxylase accompanied by inhibition of the 5′-AMP-activated protein kinase. J Biol Chem 267, 2864-2867 (1992); Dyck, J. R. et al. Regulation of 5′-AMP-activated protein kinase activity by the noncatalytic beta and gamma subunits. J Biol Chem 271, 17798-17803 (1996)).

AMPK binding to adenylate ligands (ADP, AMP) can not only increase kinase activity directly, but also decrease the ability of phospo-threonine 172 to be dephosphorylated by AMPK inhibitory phosphatases. This phosphatase protection mechanism has been described and can be measured in vitro using purified phosphorylated AMPK (Oakhill, J. S. et al. beta-Subunit myristoylation is the gatekeeper for initiating metabolic stress sensing by AMP-activated protein kinase (AMPK). Proceedings of the National Academy of Sciences of the United States of America 107, 19237-19241, doi:10.1073/pnas.1009705107 (2010); Chandrashekarappa, D. G., McCartney, R. R. & Schmidt, M. C. Ligand binding to the AMP-activated protein kinase active site mediates protection of the activation loop from dephosphorylation. J Biol Chem 288, 89-98, doi:10.1074/jbc.M112.422659 (2013)). Briefly, purified phosphorylated AMPK is added with purified protein phosphatase 2C (PP2C) and incubated for 20 minutes at 37° C., after which the reaction is stopped and the protein run on a standard quantitative western blot (Li-Cor). Compounds will be incubated with at least a 25% change in phospho-AMPKα at their IC₅₀ concentrations for initial studies as described ((Oakhill, J. S. et al. beta-Subunit myristoylation is the gatekeeper for initiating metabolic stress sensing by AMP-activated protein kinase (AMPK). Proceedings of the National Academy of Sciences of the United States of America 107, 19237-19241, doi:10.1073/pnas.1009705107 (2010); Chandrashekarappa, D. G., McCartney, R. R. & Schmidt, M. C. Ligand binding to the AMP-activated protein kinase active site mediates protection of the activation loop from dephosphorylation. J Biol Chem 288, 89-98, doi:10.1074/jbc.M112.422659 (2013)). The phospho-AMPKα surrogate measure for AMPK activity (Hawley, S. A. et al. Complexes between the LKB1 tumor suppressor, STRAD alpha/beta and MO25 alpha/beta are upstream kinases in the AMP-activated protein kinase cascade. Journal of biology 2, 28, doi:10.1186/1475-4924-2-28 (2003)) (above) will be combined with in vitro kinase and phosphatase protection data for interpretation of mechanism of action and will help stratify small molecules into different classes.

### B. Subsequent Testing

After identifying at least 3 molecules, a similarity screen followed by rational synthesis was used to generate an expanded list of positive hits containing a common structural scaffold. Routine testing for false positive hits confirmed that the expanded list of analogs included no thiol-reactive molecules and no small molecule aggregators. Kinase activity assays, phosphatase assays, and cell-intact assays were employed to determine if these analogs regulate AMPK. Of the 9 molecules tested in orthogonal assays, 4 analogs dose-dependently regulated the phosphorylation state and/or activity of purified phospho-AMPK. One analog consistently inhibited AMPK activity in both protein-based and cell-based assays. In addition to identifying novel AMPK inhibitors, these results validate the use of the fluorescence-based binding assay described herein as a tool for the discovery of new molecules that modulate AMPK activity.

### Example 4

### Dose-Dependent Regulation of AMPK

### Methods

Reagents. BAS 02250954 and BAS 03338548 were purchased from Asinex; STK823366 and STK740822 were purchased from Vitas-M Laboratory, Ltd. The remaining analogs were synthesized at North Carolina Central University, Durham, N.C. A-769662, Compound C, and ADP were purchased from Tocris, Calbiochem, and Sigma, respectively. Small molecules were dissolved in DMSO (except where otherwise noted) and stored at −20° C. Primary antibodies were purchased from Cell Signaling (except where otherwise noted); secondary antibodies were purchased from Li-Cor Biosciences. Antibodies were diluted in 1× TBS with 5% BSA, 0.1% Tween-20, 0.02% sodium azide, and 0.02% SDS (for secondary antibodies only). His-tagged AMPK. Full-length AMPK (rat His-α₁, β₁, γ₁) and regulatory fragment (mammalian His-α₁, β₂, γ₁) were purified according to published methods (Sinnett S E, Sexton J Z, Brenman J E. A High Throughput Assay for Discovery of Small Molecules that Bind AMP-activated Protein Kinase (AMPK). Current chemical genomics and translational medicine. 2013; 7:30-8. PubMed PMID: 24396733. Pubmed Central PMCID: 3854666.) Eluted regulatory fragment and full-length His-AMPK (intended for use in binding assays) were concentrated (20 mg/mL) and stored at −80° C. in 50 mM Tris (pH 8.2). Full-length AMPK intended for use in phosphatase and kinase assays was purified, mixed with GST-CaMKKβ, and allowed to react for 30 minutes at 22° C. (Tokumitsu H, Iwabu M, Ishikawa Y, Kobayashi R. Differential regulatory mechanism of Ca2+/calmodulin-dependent protein kinase kinase isoforms. Biochemistry. 2001 Nov. 20; 40(46):13925-32. PubMed PMID: 11705382.13). Concentrations of AMPK and GST-CaMKKβ in the phosphorylation reaction were 7 mg/mL and 63 μg/mL, respectively. Reaction conditions were derived from previously published methods (Chen L, Jiao Z H, Zheng L S, Zhang Y Y, Xie S T, Wang Z X, et al. Structural insight into the autoinhibition mechanism of AMP-activated protein kinase. Nature. 2009 Jun. 25; 459(7250):1146-9. PubMed PMID: 19474788.14). Glutathione Sepharose 4B resin was used to remove GST-CaMKKβ from the reaction mixture. Phosphorylated His-AMPK was further polished on a Sephacryl S-200 column, concentrated in 50 mM Tris (pH 8.2, 20 mg/mL protein), and stored at −80° C. Because Tris buffer is not recommended for isothermal titration calorimetry (ITC), full-length His-AMPK intended for ITC was purified, concentrated, and immediately frozen in 50 mM phosphate buffer (pH 7.4) (Pierce M M, Raman C S, Nall B T. Isothermal titration calorimetry of protein-protein interactions. Methods. 1999 October; 19(2):213-21. PubMed PMID: 10527727.15).

GST-calcium/calmodulin-dependent protein kinase kinase β (GST-CaMKKβ). The construct used to express GST-CaMKKβ has been previously published (Tokumitsu H, Iwabu M, Ishikawa Y, Kobayashi R. Differential regulatory mechanism of Ca2+/calmodulin-dependent protein kinase kinase isoforms. Biochemistry. 2001 Nov. 20; 40(46):13925-32. PubMed PMID: 11705382). The purification methods referenced for His-tagged AMPK were adapted for the purification of GST-CaMKKβ (Sinnett S E, Sexton J Z, Brenman J E. A High Throughput Assay for Discovery of Small Molecules that Bind AMP-activated Protein Kinase (AMPK). Current chemical genomics and translational medicine. 2013; 7:30-8. PubMed PMID: 24396733. Pubmed Central PMCID: 3854666). GST-tagged protein was eluted with 20 mM glutathione from Glutathione Sepharose 4B resin. Eluate was stored at −20° C. (10 mg/mL, 50% glycerol, 25 mM Tris pH 8.2).

GST-Acetyl CoA carboxylase (GST-ACC) peptide. Primers coding for residues surrounding Ser79 of rat ACC1 preceded by a five-residue polyglycine linker were annealed and ligated into the BamHI and XhoI sites of pGEX-4T-1. The resulting plasmid was transformed into Rosetta cells for protein expression in autoinduction media. Cell growth and lysis conditions were completed according to published methods. The lysate was centrifuged to pellet debris. Clarified supernatant was then filtered and incubated with Glutathione Sepharose 4B resin. The peptide was eluted with 20 mM glutathione in lysis buffer. Eluate was concentrated and buffer-exchanged into 50 mM Tris-Cl (pH 8.2) before being stored at 20 mg/mL at −80° C. The peptide sequence (including the polyglycine linker) added to the GST tag was GGGGGLAFHMRSSMSGLHLVKQGR-DRKK. Ser79, which is phosphorylated by AMPK, is underlined.

His-protein phosphatase 2C alpha (His-PP2Cα). His-PP2Cα was purified according to the methods referenced above for His-tagged AMPK and then stored (17 mg/mL) at −20° C. in 50% glycerol, 25 mM Tris (pH 8.2) (4). This construct has been previously published (16).

Binding assays. Except where otherwise noted, assay assembly and data analysis were completed according to published methods. Binding assays were manually assembled, with the exception of the similarity screen. Automated assembly of the similarity screen was completed according to published methods. Analogs were cherry-picked from a 10 mM Asinex small molecule library and a 0.5 mM kinase focused library, which has been described previously (4). Molecules were screened at a final concentration of 45 and 2.3 μM, respectively.

ITC. A MicroCal Auto-iTC200 system (GE Healthcare, Pittsburgh, Pa.) was used to study binding interactions between ADP and full-length His-AMPK at room temperature in the presence of 5 mM phosphate buffer (pH 7.4). To decrease the ionic strength of the buffer, full-length AMPK frozen in 50 mM phosphate buffer was thawed, diluted in 5 mM phosphate buffer (pH 7.4) and then concentrated via centrifugal filtration. At the start of the ITC experiment, the concentrations of His-AMPK (in the sample cell) and ADP (in the syringe) were 70 μM and 1.4 mM, respectively.

Kinase assays. Assay conditions were adapted from published methods (17). The final assay buffer was comprised of 40 mM Hepes (pH 7.4) 5 mM MgCl₂, 200 μM ATP, 75 mM NaCl, 0.01% Triton X-100, 1 mM DTT, and 0.45% DMSO (vehicle). Because some analogs had poor solubility at high concentrations, all analogs and controls were diluted in assay buffer and centrifuged (1 min, 5000 rpm, 4° C.) to remove insoluble matter. GST-ACC peptide and p-AMPK were thawed, diluted in assay buffer, and then added to the supernatant at final concentrations of 900 and 0.1 ng/μL, respectively. Kinase assays were assembled on ice; reactions were allowed to proceed for 30 minutes at room temperature. Reactions were stopped by the addition of loading buffer and 48 mM DTT followed promptly by freezing at −20° C.

Phosphatase assays. Assay conditions were adapted from published methods (18). In short, the final assay buffer was compromised of 50 mM Tris (pH 7.5), 2.5 MgCl₂, 0.01% Triton X-100, 1 mM DTT, and 0.45% DMSO (vehicle). Because some analogs had poor solubility at high concentrations, all analogs and controls were diluted in assay buffer and centrifuged (1 min, 5000 rpm, 4° C.) to remove insoluble matter. PP2C and p-AMPK were added to the supernatant at final concentrations of 2 and 3 ng/μL, respectively. Phosphatase reactions were allowed to proceed for 30 minutes at 37° C. Reactions were stopped by the addition of loading buffer and 48 mM DTT followed promptly by freezing at −20° C.

Cell-intact assays. HEK cells were grown in DMEM with 10% FBS and 1% penicillin/streptomycin at 37° C. A subset of plates was serum-starved for 5 hours on the day of the experiment. After 5 hours of serum-starvation, cells were treated with either controls (0.2% DMSO, A-769662, or Compound C) or small molecules in the absence of serum for an additional hour prior to lysis. Another subset of plates was serum-starved for 6 hours and then treated with either controls or small molecules for 1 hour in the presence of 10% FBS. A time course of both AMPK and ACC phosphorylation in HEK cells during serum starvation has already been published (Pirkmajer S, Chibalin A V. Serum starvation: caveat emptor. American journal of physiology Cell physiology. 2011 August; 301(2):C272-9. PubMed PMID: 2161361).

After incubating in the presence of drugs for one hour, cells were washed three times with ice-cold PBS and lysed according to published methods (Kazgan N, Williams T, Forsberg L J, Brenman J E. Identification of a nuclear export signal in the catalytic subunit of AMP-activated protein kinase. Molecular biology of the cell. 2010 Oct. 1; 21(19):3433-42. PubMed PMID: 20685962. Pubmed Central PMCID: 2947478). Lysates were centrifuged at 16,000 g (10 minutes, 4° C.). Clarified supernatant (30 μL) was then transferred to 10 μL 4X loading buffer and 2 μL 1 M DTT. Samples were immediately frozen at −20° C. The concentration of protein in diluted clarified supernatant was calculated using a DC protein assay kit (BioRad) prior to western analyses.

Western analyses. Protein samples were boiled for 15 minutes in loading buffer and DTT prior to SDS-PAGE. Blots for protein-based assays were sequentially incubated with the following: 5% BSA in 1× TBS (1 hr, 22° C.), 1:1000 rabbit anti-human p-AMPK-α antibody or p-ACC antibody (2 hr, 22° C.), and 1:10,000 donkey anti-rabbit secondary antibody (1 hr, 22° C.). Blots for phosphatase assays were probed a second time using 1:100 mouse anti-human total AMPK antibody (overnight, 4° C.; from UNC Antibody Core Facility) followed by 1:10,000 donkey anti-mouse secondary antibody (1 hr, 22° C.). Blots for clarified cell lysates were sequentially probed for p-AMPK (1:500 dilution, overnight at 4° C. or 4 hours at 22° C.), total ACC (1:1000 dilution, overnight at 4° C. or 4 hours at 22° C.), and tubulin (1:10,000 dilution, 1 hr, 22° C.); a parallel set of blots were probed for p-ACC (1:500 dilution, overnight at 4° C. or 4 hours at 22° C.), total AMPK (1:1000 dilution, overnight at 4° C. or 4 hours at 22° C.), and tubulin (1:10,000 dilution, 1 hr, 22° C.). Because phosphorylated and total protein were imaged on separate membranes, the signals were normalized to the internal tubulin signal prior to calculating the ratio of phosphorylated to total protein. Scanning and imaging were completed according to published methods.

Data analysis. GraphPad Prism was used to fit dose response data for binding assays. Instant JChem (ChemAxon LLC, Cambridge, Mass.) was used to curate data for over 13,000 molecules that were tested herein and in a previous publication.

Thiol-reactivity. Binding assays were repeated in the presence and absence of DTT (Table 1). STK740822 produced similar binding curves in the presence of 0-1 mM DTT, suggesting that STK740822 is not thiol-reactive (Table 3). STL035166, however, provided a dramatically different dose response curve in the presence of 1 mM DTT, with an extrapolated 1050 of ˜250 μM (Table 3). Because thiol-reactive molecules may be promiscuous and therefore undesirable for drug development, subsequent orthogonal assays were limited to the characterization of STK740822 and its structural analogs.

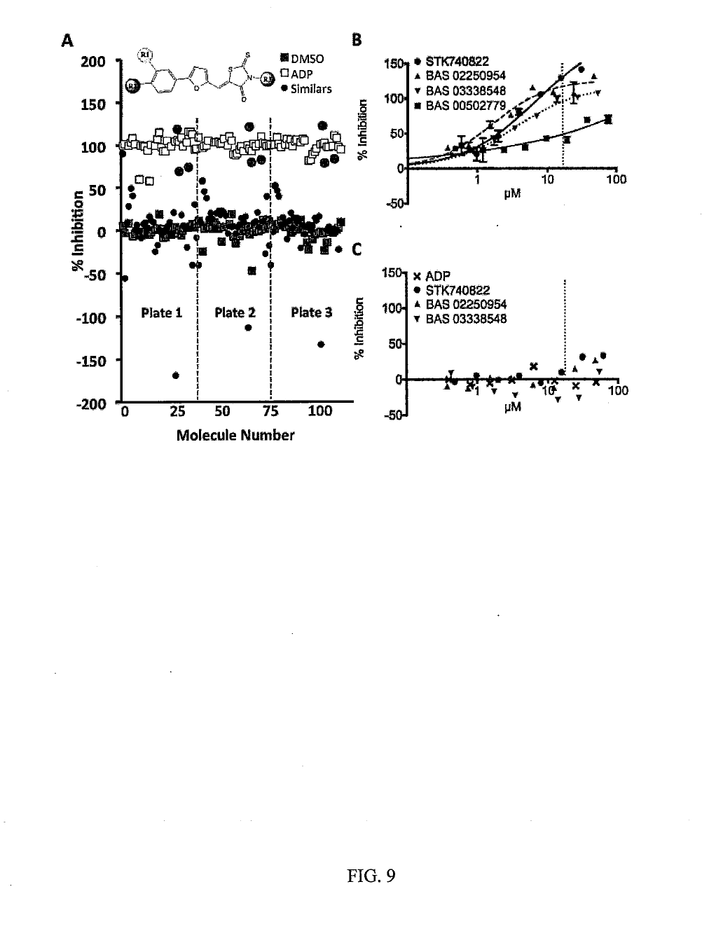

To identify analogs of STK740822 and improve the likelihood of discovering a novel AMPK modulator, a small-scale, automated similarity screen of molecules containing sub-structural scaffolds present in the parent molecule STK740822 was conducted (FIG. 9A). Three molecules (BAS 02250954, BAS 03338548, and BAS 00502779) reproducibly inhibited the protein-bound signal of MANT-ADP (FIG. 9A). To confirm these hits, the binding IC50s in the presence of full-length AMPK (FIG. 9B) was investigated again. BAS 02250954 and BAS 0333858 dose-dependently inhibited MANT-ADP's protein-bound signal with IC50s that were less than 5 μM. At high concentrations, however, these molecules inhibited MANT-ADP's fluorescent signal by more than 100%, suggesting that these molecules may decrease MANT-ADP's signal by directly interacting with the probe.

To determine if these molecules were directly interacting with the probe, binding assays were repeated with 0 μM AMPK (FIG. 9C). At concentrations approaching 100 μM, these molecules interfered slightly with MANT-ADP's signal. Extremely high concentrations of these molecules, however, would not be tested in protein-based or cell-based assays. Analog testing was continued in subsequent orthogonal assays with the intention of highlighting molecules that regulate AMPK activity at low doses.

To rationally synthesize additional analogs, an R group decomposition table was generated that ranked positive and negative hits from the similarity screen and a previously published high-throughput screen. Molecules were ranked according to binding IC50s. The R group decomposition analysis revealed that non-thiol-reactive positive hits had a common structural scaffold consisting of rhodanine and phenyl-furan moieties (FIG. 9A inset). To generate new hits, additional analogs containing this scaffold were synthesized. Synthesized analogs inhibited binding of MANT-ADP in the presence of full-length AMPK and the regulatory fragment (Tables 4 and 5).

To validate the methods for chemical synthesis, STK740822 was synthesized and tested in a binding assay with full-length AMPK. The synthesized control produced a similar binding IC50 (9 μM) and Hill Slope (1.2) compared to those of the commercially available molecule (Table 4). The panel of analogs yielded similar binding curves in the presence of 0-1 mM DTT (Table 4). Both 0 and 1 mM DTT conditions were tested in parallel on the same plate. Likewise, molecules yielded similar binding curves in the presence of 0-0.01% Triton X-100 (tested in parallel on the same plate) (Table 5). Thus, these analogs are most likely neither thiol-reactive nor small molecule aggregators.

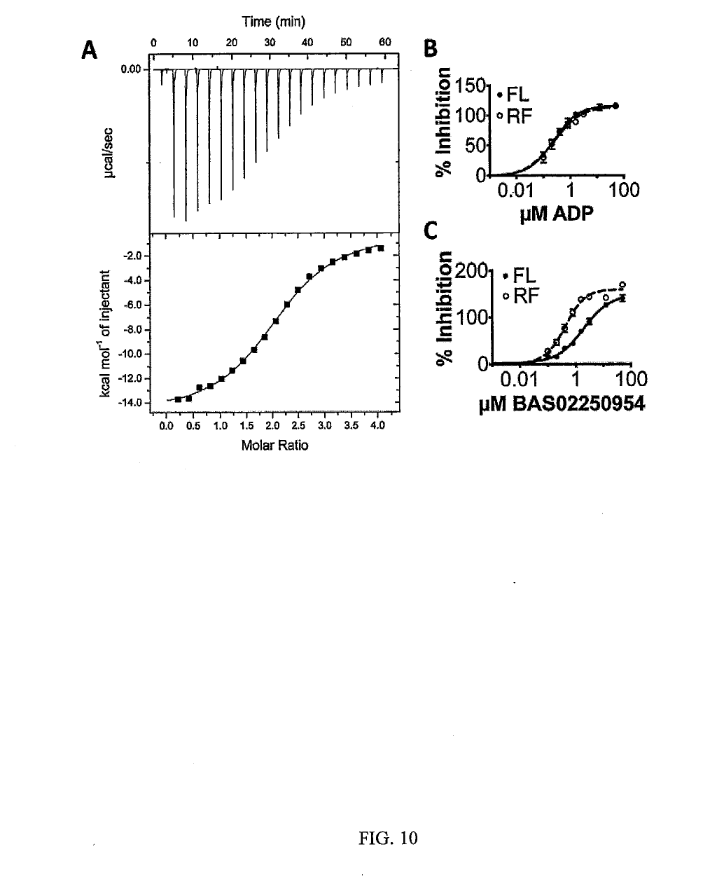

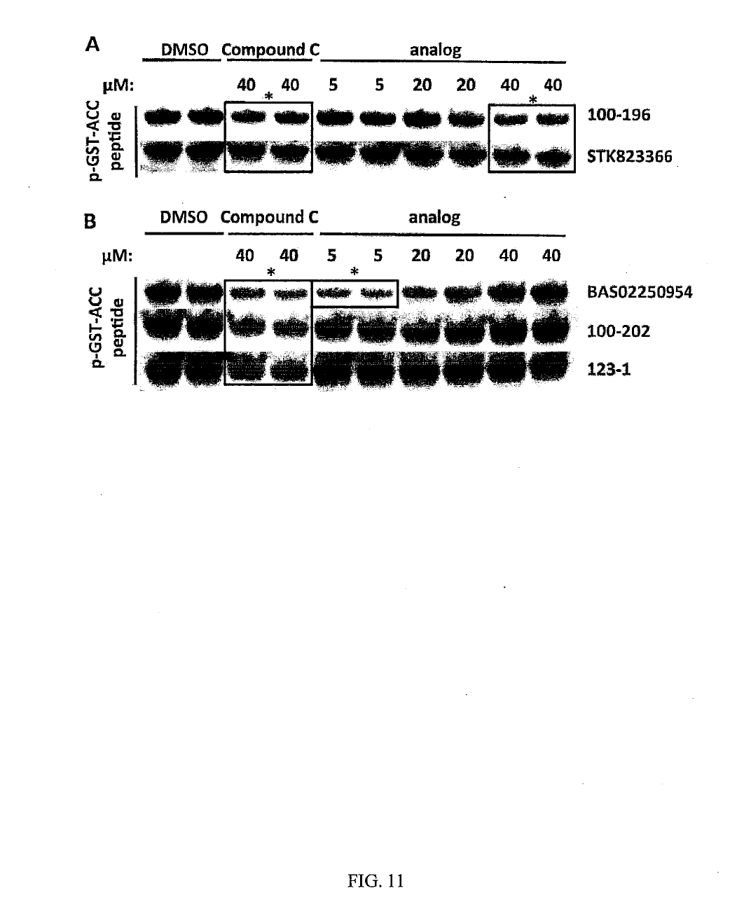

Many analogs exhibited much lower binding IC50s in the presence of the regulatory fragment (Tables 4 and 5). The positive control ADP, which binds two sites on AMPK-γ, has the same binding IC50 for both full-length AMPK and the regulatory fragment in the MANT-ADP assay (FIG. 10A-B). Two analogs (100-196, and STK823366) dose-dependently inhibited the activity of purified p-AMPK (FIG. 11A). At 40 μM, these 2 analogs significantly decreased the yield of p-GST-ACC peptide (p≦0.05, compared to DMSO control) and achieved a level of inhibition similar to that of 40 μM Compound C, a promiscuous kinase inhibitor. Interestingly, the analog BAS 02250954 significantly inhibited substrate phosphorylation at low doses (p≦0.05, compared to DMSO control), but permitted substrate phosphorylation at higher doses (FIG. 11B). When repeated, with an additional concentration at 1 μM, a complete U-shaped dose response with high levels of p-GST-ACC peptide produced in the presence of both 1 and 40 μM BAS 02250954 was observed (data not shown). Two other analogs (100-202, 123-1) appeared to dose-dependently regulate AMPK activity, but not significantly. The remainder of the analogs tested had no dose-dependent effects on AMPK activity at 5, 20, or 40 μM (data not shown).

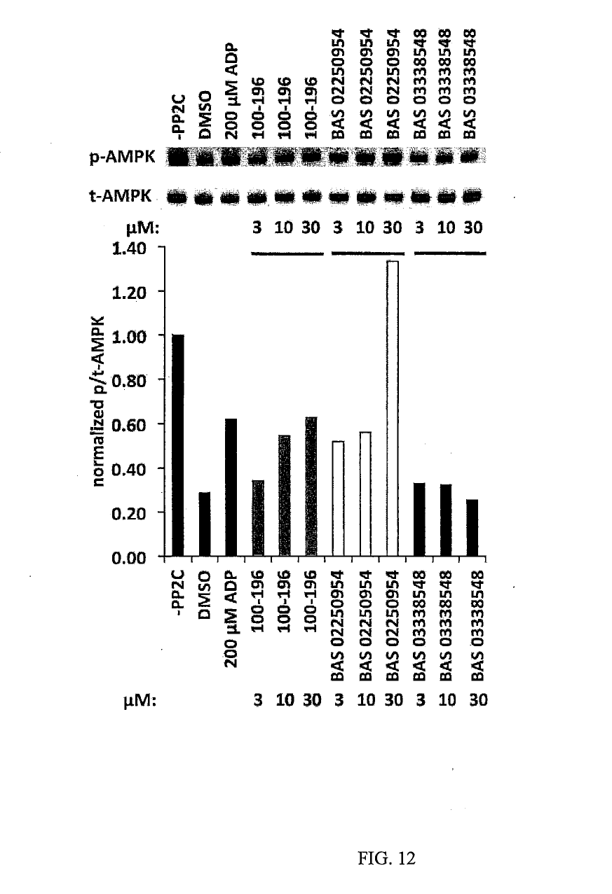

The U-shaped dose response curve for BAS 02250954 suggested that the analog may exert multiple regulatory effects that oppose each other. To determine if BAS 02250954 could protect p-AMPK from dephosphorylation, the analog was tested in the presence of both p-AMPK and PP2C and then quantified p-AMPK and t-AMPK levels via western analyses (FIG. 12). After calculating the ratio of the signal intensities for p-AMPK and total AMPK, the ratios were normalized to that of the no-PP2C control. Both BAS 02250954 and its closest structural analog, 100-196, protected p-AMPK from dephosphorylation (FIG. 12).

Furthermore, the level of protection provided by these two analogs was greater than or similar to that provided by 200 μM ADP. In addition, BAS 02250954 protected p-AMPK at doses that were also shown to restore p-AMPK activity in the in vitro kinase assays. In contrast, the more distant structural analog BAS 03338548 failed to protect p-AMPK from dephosphorylation (FIG. 12). None of the remaining analogs from Table 4 protected p-AMPK when tested at 3, 10, and 30 μM (data not shown). The in vitro data collected thus far suggested that the dose-dependent modulators identified in FIG. 11 may regulate AMPK via multiple regulatory mechanisms and perhaps via multiple binding sites.

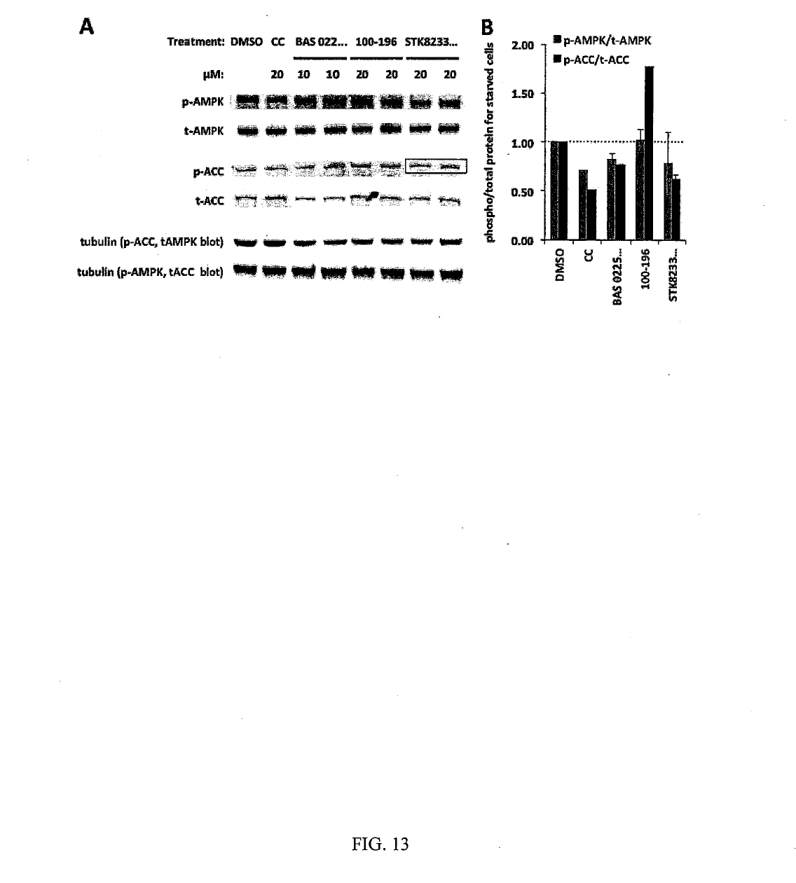

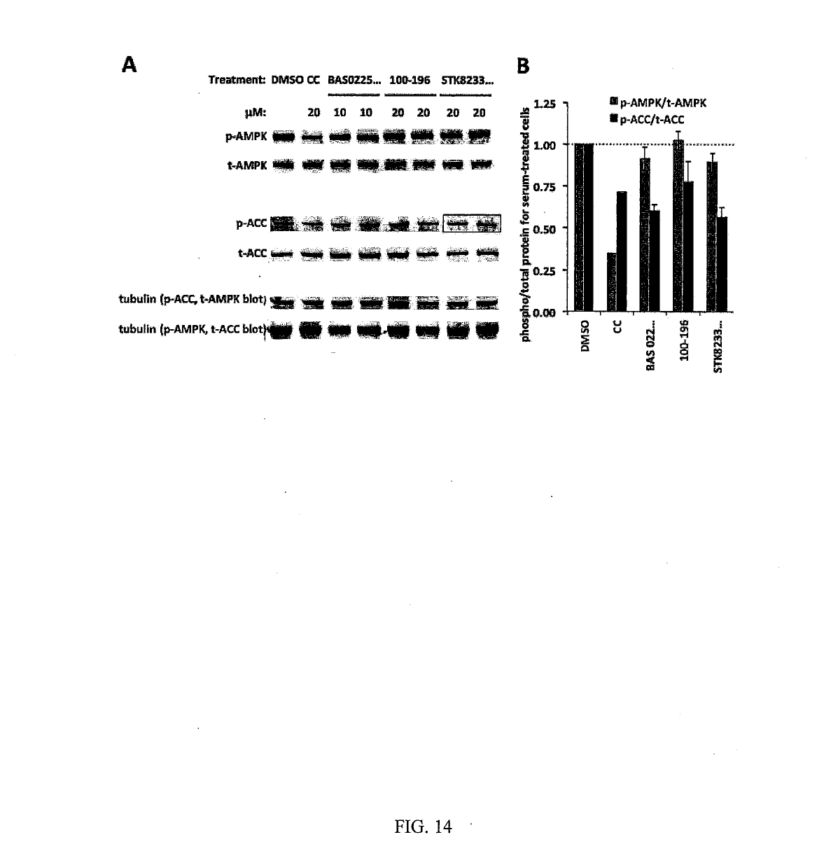

To determine if analogs regulate AMPK activity in cells, HEK cells cultured in media conditions that induce high, moderate, or low p-AMPK levels in the absence of drugs were tested. Serum-starved HEK cells have high endogenous AMPK activity and are ideal for identifying novel inhibitors. In contrast, HEK cells continuously cultured in the presence of 10% serum have moderate AMPK activity, showing modest decreases in p-ACC levels upon treatment with the non-specific AMPK inhibitor Compound C. STK823366 decreased phosphorylation of the AMPK substrate ACC in serum-starved cells and serum-treated cells (FIGS. 13-14). The positive control, Compound C, achieved the best inhibition in serum-starved cells, in contrast to serum-treated cells (FIGS. 13-14). Similar to STK823366, BAS 02250954 decreased substrate phosphorylation in serum-treated cells (FIG. 14). In the absence of serum, however, treatment with BAS 02250954 caused many cells to detach from tissue culture plates.

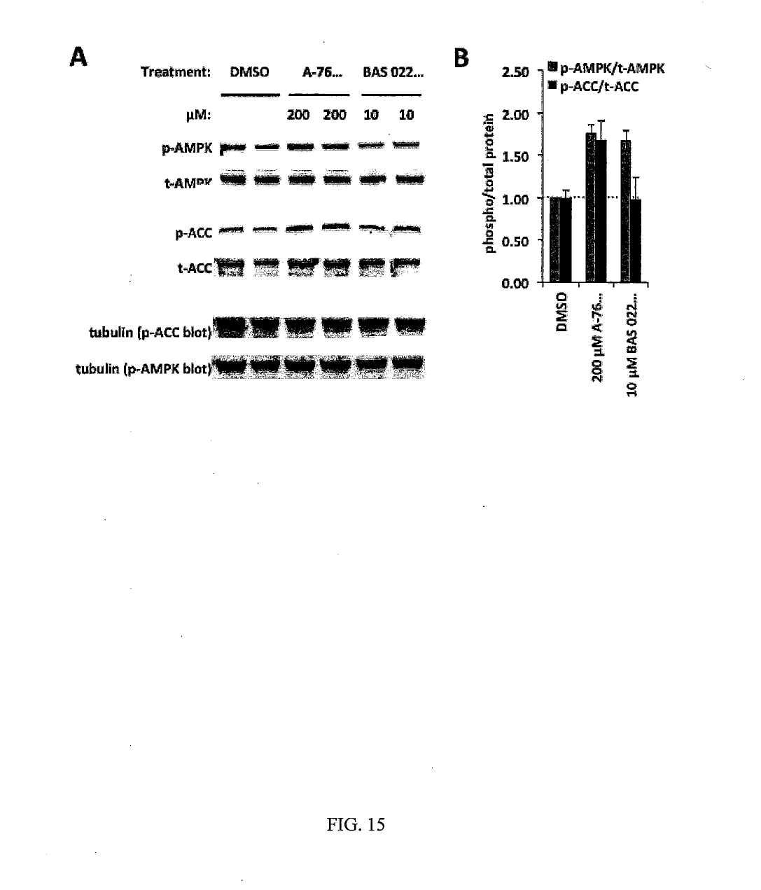

BAS 02250954 was tested on cells that were subjected to a cycle of serum starvation followed by restoration of 10% serum (FIG. 15). Cells that have been pre-conditioned in this manner have extremely low p-AMPK levels and are ideal for the identification of novel activators. Because BAS 02250954 inhibited purified p-AMPK activity but protected purified p-AMPK from dephosphorylation, we expected this analog to de-couple AMPK phosphorylation from activation in pre-conditioned HEK cells (FIGS. 11, 12, 15). Pre-conditioned HEK cells treated with BAS 02250954 for 1 hour in the presence of serum had high levels of p-AMPK with no change in substrate phosphorylation. In contrast, the AMP-mimetic A-769662 increased both p-AMPK and p-ACC levels.

Whereas BAS 02250954 and STK823366 gave consistent results in both protein-based and cell-based assays, analog 100-196 unexpectedly increased levels of p-ACC in serum-starved HEK cells (FIG. 13). The failure of 100-196 to consistently inhibit substrate phosphorylation in both protein-based and cell-based assays further supports the characterization of STK823366 in future cell-based studies and the use of STK823366 as a tool compound for protein-based assays.

### Conclusion

Analogs of STK823366, a molecule that displaces a fluorescent ADP analog from binding to the regulatory region of AMPK, can inhibit the activity of both purified His-AMPK and endogenous AMPK in intact HEK cells.

### Example 5

### Compound Synthesis

In the following synthesis examples, all chromatography was performed on a Teledyne-ISCO Combiflash R_{f} system unless otherwise noted. LC/MS was performed using a Waters Sunfire OBD C13 3×100 mm, 5 μM column with a flow rate of 0.5 mL/min. The solvent system used was either 10 to 95% or 70 to 95% acetonitrile/water/0.1% formic acid over a 7 min. elution time. The NMR analysis was performed on a Varian 500 mHz NMR.

### Example A

### 2-{(5Z)-5-{[5-(4-Chlorophenyl)furan-2-yl]methylidene}-4-oxo-2-sulfanylidene-1,3-thiazolidin-3-yl}acetic acid

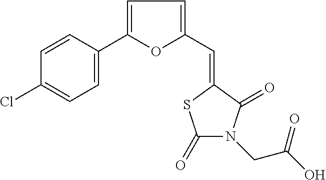 [SMILES: O=C(O)CN1C(=O)S/C(=C\c2ccc(-c3ccc(Cl)cc3)o2)C1=O]

This compound was prepared in a similar fashion as described in Bioorg. Med. Chem. 17, (2009) 2576-2584 as follows: A mixture of 5-(4-chlorophenyl)furan-2-carbaldehyde (0.2 g, 1.0 mmol) [ACROS Chemicals], rhodanine-3-acetic acid (0.19 g, 1.0 mmol) [TCI] and anhydrous sodium acetate (0.25 g, 3.0 mmol) in acetic acid (3 mL) was heated at 120° C. for 5 h. The reaction mixture was cooled to ambient temperature and the resulting solid was filtered and washed repeatedly with diethyl ether. After air drying, 0.34 g (89.5% yield) of 2-{(5Z)-5-{[5-(4-chlorophenyl)furan-2-yl]methylidene}-4-oxo-2-sulfanylidene-1,3-thiazolidin-3-yl}acetic acid was obtained as a reddish orange solid.

MS: m/z 378.0 (MH⁻).

¹H NMR (500 MHz, DMSO-d₆): δ 4.637 (s, 2H), 7.405 (s, 2H), 7.656 (d, 2H), 7.741 (s, 1H), 7.885 (d, 2H).

### Example B

### 2-{(5Z)-5-{[5-(4-Methylphenyl)furan-2-yl]methylidene}-4-oxo-2-sulfanylidene-1,3-thiazolidin-3-yl}acetic acid (STK740822)

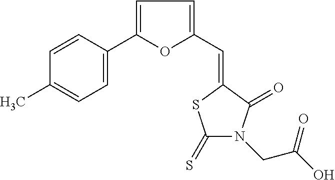 [SMILES: Cc1ccc(-c2ccc(/C=C3\SC(=S)N(CC(=O)O)C3=O)o2)cc1]

### Step 1: 5-(4-methylphenyl)furan-2-carbaldehyde

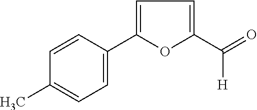

5-(4-Methylphenyl)furan-2-carbaldehyde: This compound was prepared in a similar fashion as described in WO2011/135303A2, Method B, page 87 as follows: To a solution of p-tolylboronic acid (0.82 g, 6.0 mmol) [ACROS Chemicals] and 5-bromo-2-furaldehyde (1.0 g, 6.0 mmol) [ACROS Chemicals] in 40 mL of a 4:1 mixture of toluene and ethanol at ambient temperature was added Palladium II acetate (67 mg, 0.3 mmol), triphenylphosphine (0.31 g, 1.2 mmol) and saturated aqueous sodium bicarbonate solution (6.7 mL). The mixture was heated at 80° C. for 22 h. After cooling to ambient temperature, the reaction mixture was washed with water, then brine and dried over sodium sulfate. The solution was filtered, silica gel was added to the filtrate and the mixture was concentrated in vacuo. The residue was transferred to a pre-column and purified by chromatography using initially hexanes (5 min.) as eluent. The eluent was modified to 10% ethyl acetate/hexanes over a 11 min. period and held at 10% ethyl acetate/hexanes for the remainder of the purification. The fractions containing the pure major component were combined and concentrated in vacuo to give 0.93 g (83% yield) of 5-(4-methyl phenyl)furan-2-carbaldehyde as an orange oil. MS: m/z 187.1 (MH⁺).

Step 2: 2-{(5Z)-5-{[5-(4-Methylphenyl)furan-2-yl]methylidene}-4-oxo-2-sulfanylidene-1,3-thiazolidin-3-yl}acetic acid: This compound was prepared in a similar fashion as described in Example 1 substituting 5-(4-methylphenyl)furan-2-carbaldehyde for 5-(4-chlorophenyl)furan-2-carbaldehyde and heating the reaction mixture for 6 h to give 2-{(5Z)-5-{[5-(4-methylphenyl)furan-2-yl]methylidene}-4-oxo-2-sulfanylidene-1,3-thiazolidin-3-yl}acetic acid in a 95.6% yield as a red solid.

MS: m/z 358 (MH⁻) and m/z 360 (MH⁺).

¹H NMR (500 MHz, DMSO-d₆): δ 2.379 (s, 3H), 4.668 (s, 2H), 7.301 (d, 1H), 7.399 (m, 3H), 7.740 (s, 1H), 7.780 (d, 2H).

### Example C

### 2-{(5Z)-5-{[5-(4-Methoxyphenyl)furan-2-yl]methylidene}-4-oxo-2-sulfanylidene-1,3-thiazolidin-3-yl}acetic acid

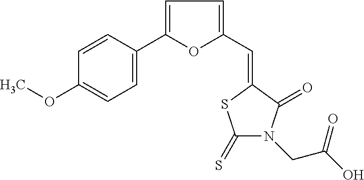 [SMILES: COc1ccc(-c2ccc(/C=C3\SC(=S)N(CC(=O)O)C3=O)o2)cc1]

### Step 1, 5-(4-Methoxyphenyl)furan-2-carbaldehyde

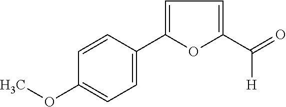

To a solution of 4-methoxyphenylboronic acid (0.91 g, 6.0 mmol) [ACROS Chemicals] and 5-bromo-2-furaldehyde (1.0 g, 6.0 mmol) in 40 mL of a 4:1 mixture of toluene and ethanol at ambient temperature was added Palladium II acetate (67 mg, 0.3 mmol), triphenylphosphine (0.31 g, 1.2 mmol) and saturated aqueous sodium bicarbonate solution (6.7 mL). The mixture was heated at 80° C. for 21.5 h. After cooling to ambient temperature, the reaction mixture was poured into a mixture of ethyl acetate and water. The aqueous layer was extracted with ethyl acetate and the combined organic phase was washed with water, then brine and dried over sodium sulfate. The solution was filtered, silica gel was added to the filtrate and the mixture was concentrated in vacuo. The residue was transferred to a pre-column and purified by chromatography using initially hexanes (5 min.) as eluent. The eluent was modified to 10% ethyl acetate/hexanes over a 10 min. period and held at 10% ethyl acetate/hexanes for the remainder of the purification. The fractions containing the pure major component were combined and concentrated in vacuo to give 0.96 g (79% yield) of 5-(4-methoxyphenyl)furan-2-carbaldehyde as an orange viscous oil. See Bioorg. Med. Chem. 12 (2004) 4585-4600.

MS: m/z 203.1 (MH⁺).

¹H NMR (500 MHz CDCl₃): δ 2.859 (s, 3H), 6.713 (d, 1H), 6.964 (d, 2H), 7.300 (d, 1H), 7.766 (d, 2H), 9.603 (s, 1H).

Step 2: 2-{(5Z)-5-{[5-(4-methoxyphenyl)furan-2-yl]methylidene}-4-oxo-2-sulfanylidene-1,3-thiazolidin-3-yl}acetic acid: This compound was prepared in a similar fashion as described in Example 1 substituting 5-(4-methoxyphenyl)furan-2-carbaldehyde for 5-(4-chlorophenyl)furan-2-carbaldehyde and heating the reaction mixture for 6 h to give a 86.8% yield of 2-{(5Z)-5-{[5-(4-methoxyphenyl)furan-2-yl]methylidene}-4-oxo-2-sulfanylidene-1,3-thiazolidin-3-yl}acetic acid as a red solid. See European Journal of Medicinal Chemistry 61 (2013) 104-115.

MS: m/z 376 (MH⁺).

¹H NMR (500 MHz, DMSO-d₆): δ 2.827 (s, 3H), 4.513 (s, 2H), 7.148 (d, 2H), 7.195 (d, 1H), 7.353 (d, 1H), 7.665 (s, 1H), 7.811 (d, 2H).

### Example D

### 2-{(5Z)-5-{[5-(3,4-Dichlorophenyl)furan-2-yl]methylidene}-4-oxo-2-sulfanylidene-1,3-thiazolidin-3-yl}acetic acid

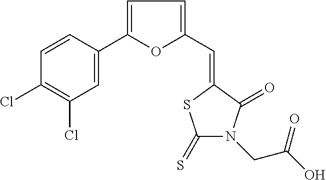 [SMILES: O=C(O)CN1C(=O)/C(=C/c2ccc(-c3ccc(Cl)c(Cl)c3)o2)SC1=S]

### Step 1, 5-(3,4-Dichlorophenyl)furan-2-carbaldehyde

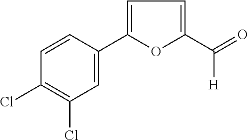 [SMILES: O=Cc1ccc(-c2ccc(Cl)c(Cl)c2)o1]

To a solution of 3,4-dichlorophenyl boronic acid (1.1 g, 6.0 mmol)[ACROS Chemicals] and 5-bromo-2-furaldehyde (1.0 g, 6.0 mmol) in 40 mL of a 4:1 mixture of toluene and ethanol at ambient temperature was added Palladium II acetate (67 mg, 0.3 mmol), triphenylphosphine (0.31 g, 1.2 mmol) and saturated aqueous sodium bicarbonate solution (6.7 mL). The mixture was heated at 80° C. for 22 h. After cooling to ambient temperature, the reaction mixture was partitioned between toluene and water. The aqueous layer was extracted with toluene and the combined organic phase was washed with water, then brine and dried over sodium sulfate. The solution was filtered, silica gel was added to the filtrate and the mixture was concentrated in vacuo. The residue was transferred to a pre-column and purified by chromatography using initially hexanes (5 min.) as eluent. The eluent was modified to 10% ethyl acetate/hexanes over an 11 min. period and held at 10% ethyl acetate/hexanes for the remainder of the purification. Several of the fractions containing the pure major component produced crystals. The solid (A) was filtered and air dried to give 0.18 g of 5-(3,4-dichlorophenyl)furan-2-carbaldehyde (See WO 01/39773A1) as a white solid. The combined filtrate from (A) with the remaining fractions containing the pure major component was concentrated in vacuo to give 0.65 g of (B) [5-(3,4-dichlorophenyl)furan-2-carbaldehyde] as a pale yellow solid. (A) and (B) exhibited the same R_{f} on silica gel TLC using 10% ethyl acetate/hexanes (R_{f}=0.29). Total yield=0.83 g (57% yield)

MS: of (A) and (B) m/z 241.0 (MH⁺).

¹H NMR (500 MHz DMSO-d₆) of (A): δ 7.464 (d, 1H), 7.676 (d, 1H), 7.788 (d, 1H), 7.855 (dd, 1H), 8.145 (6, 1H), 9.647 (s, 1H).

Step 2: 2-{(5Z)-5-{[5- (3,4-Dichlorophenyl)furan-2-yl]methylidene}-4-oxo-2-sulfanylidene-1,3-thiazolidin-3-yl}acetic acid: This compound was prepared in a similar fashion as described in Example 1 substituting 5-(3,4-dichlorophenyl)furan-2-carbaldehyde for 5-(4-chlorophenyl)furan-2-carbaldehyde and heating the reaction mixture for 5.5 h. The filtered solid was washed with water, then diethyl ether to give a 80.2% yield of 2-{(5Z)-5-{[5-(3,4-dichlorophenyl)furan-2-yl]methylidene}-4-oxo-2-sulfanylidene-1,3-thiazolidin-3-yl}acetic acid as an orange solid. See WO20 0010573A1.

MS: m/z 411.9 (MH⁻).

¹H NMR (500 MHz, DMSO-d₆): δ 4.739 (s, 2H), 7.418 (d, 1H), 7.517 (d, 1H), 7.772 (s, 1H), 7.809 (d, 1H), 7.864 (d, 1H), 8.115 (s, 1H), 13.423 (s, 1H).

### Example E

### 2-{(5Z)-5-{[5-(3-Methylphenyl)furan-2-yl]methylidene}-4-oxo-2-sulfanylidene-1,3-thiazolidin-3-yl}acetic acid

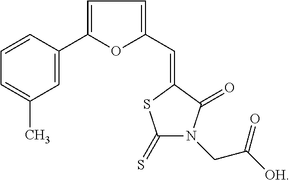 [SMILES: Cc1cccc(-c2ccc(/C=C3\SC(=S)N(CC(=O)O)C3=O)o2)c1]

### Step 1, 5-(3-methylphenyl)furan-2-carbaldehyde

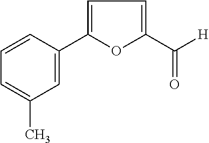

To a solution of 3-methylphenyl boronic acid (0.82 g, 6.0 mmol) [ACROS Chemicals] and 5-bromo-2-furaldehyde (1.0 g, 6.0 mmol) in 40 mL of a 4:1 mixture of toluene and ethanol at ambient temperature was added Palladium II acetate (67 mg, 0.3 mmol), triphenylphosphine (0.31 g, 1.2 mmol) and saturated aqueous sodium bicarbonate solution (6.7 mL). The mixture was heated at 80° C. for 18 h. After cooling to ambient temperature, the reaction mixture was washed twice with water, then brine and dried over sodium sulfate. The solution was filtered, silica gel was added to the filtrate and the mixture was concentrated in vacuo. The residue was transferred to a pre-column and purified by chromatography using initially hexanes (3 min.) as eluent. The eluent was modified to 10% ethyl acetate/hexanes over a 6 min. period and held at 10% ethyl acetate/hexanes for the remainder of the purification. The fractions containing the pure desired product were combined and concentrated in vacuo to give 0.91 g (81% yield) of 5-(3-methylphenyl)furan-2-carbaldehyde as a gold oil. See ChemMedChem 2009, 4, 809-819.

MS: m/z 187.1 (MH⁺).

¹H NMR (500 MHz DMSO-d₆): δ 2.364 (s, 3H), 7.253 (d, 1H), 7.252 (dm, 1H), 7.381 (t, 1H), 7.361 (d, 1H), 7.655 (dm, 1H), 7.688 (m, 1H), 9.590 (s, 1H).

Step 2: 2-{(5Z)-5-{[5-(3-methylphenyl)furan-2-yl]methylidene}-4-oxo-2-sulfanylidene-1,3-thiazolidin-3-yl}acetic acid: This compound was prepared in a similar fashion as described in Example 1 substituting 5-(3-methylphenyl)furan-2-carbaldehyde for 5-(4-chlorophenyl)furan-2-carbaldehyde. The filtered solid was washed repeatedly with water, then diethyl ether to give a 82.3% yield of 2-{(5Z)-5-{[5-(3-methyl phenyl)furan-2-yl]methylidene}-4-oxo-2-sulfanylidene-1,3-thiazolidin-3-yl}acetic acid as a reddish orange solid.

MS: m/z 358 (MH⁻).

¹H NMR (500 MHz, DMSO-d₆): δ 2.497 (s, 3H), 4.723 (s, 2H), 7.268(d, 1H), 7.330 (d, 1H), 7.407 (d, 1H), 7.464 (t, 1H), 7.665 (d, 1H) 7.695 (s, 1H), 7.757 (s, 1H), 13.408 (s, 1H).

### Example F

### 2-{(5Z)-5-{[5-(2-Methylphenyl)furan-2-yl]methylidene}-4-oxo-2-sulfanylidene-1,3-thiazolidin-3-yl}acetic acid

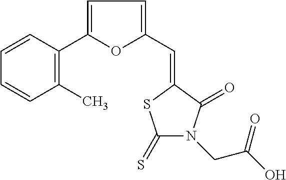 [SMILES: Cc1ccccc1-c1ccc(/C=C2\SC(=S)N(CC(=O)O)C2=O)o1]

### Step 1, 5-(2-methylphenyl)furan-2-carbaldehyde

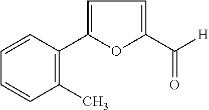

To a solution of 2-methylphenyl boronic acid (1.0 g, 7.0 mmol) [ACROS Chemicals] and 5-bromo-2-furaldehyde (1.3 g, 7.0 mmol) in 40 mL of a 4:1 mixture of toluene and ethanol at ambient temperature was added Palladium II acetate (79 mg, 0.35 mmol), triphenylphosphine (0.37 g, 1.4 mmol) and saturated aqueous sodium bicarbonate solution (7.8 mL). The mixture was heated at 80° C. for 18 h. After cooling to ambient temperature, the reaction mixture was washed with water, then brine and dried over sodium sulfate. The solution was filtered, silica gel was added to the filtrate and the mixture was concentrated in vacuo. The residue was transferred to a pre-column and purified by chromatography using initially hexanes (3 min.) as eluent. The eluent was modified to 10% ethyl acetate/hexanes over a 6 min. period and held at 10% ethyl acetate/hexanes for the remainder of the purification. The fractions containing the pure desired product were combined and concentrated in vacuo to give 1.13 g (86.9% yield) of 5-(2-methylphenyl)furan-2-carbaldehyde as a gold oil. See WO2009041705 (A2).

MS: m/z 187.1 (MH⁺).

¹H NMR (500 MHz DMSO-d₆): δ 2.493 (s, 3H), 7.054 (d, 1H), 7.337 (m, 2H), 7.345 (d, 1H), 7.649 (d, 1H), 7.742 (dm, 1H), 9.616 (s, 1H).

Step 2: 2-{(5Z)-5-{[5-(2-methylphenyl)furan-2-yl]methylidene}-4-oxo-2-sulfanylidene-1,3-thiazolidin-3-yl}acetic acid: This compound was prepared in a similar fashion as described in Example 1 substituting 5-(2-methylphenyl)furan-2-carbaldehyde for 5-(4-chlorophenyl)furan-2-carbaldehyde. The filtered solid was washed repeatedly with water, then diethyl ether to give a 54.2% yield of 2-{(5Z)-5-{[5-(2-methylphenyl)furan-2-yl]methylidene}-4-oxo-2-sulfanylidene-1,3-thiazolidin-3-yl}acetic acid as an orange solid.

MS: m/z 358 (MH⁻).

¹H NMR (500 MHz, DMSO-d₆): δ 2.533 (s, 3H), 4.728 (s, 2H), 7.113(d, 1H), 7.380 (m, 2H), 7.429 (m, 2H), 7.782 (s, 1H), 7.801 (d, 1H), 13.404 (s, 1H).

### Example G

### 2-{(5Z)-5-{[5-(3,4-difluorophenyl)furan-2-yl]methylidene}-4-oxo-2-sulfanylidene-1,3-thiazolidin-3-yl}acetic acid

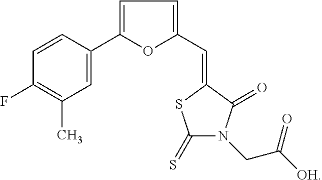 [SMILES: Cc1cc(-c2ccc(/C=C3\SC(=S)N(CC(=O)O)C3=O)o2)ccc1F]

### Step 1: 5-(3,4-difluorophenyl)furan-2-carbaldehyde

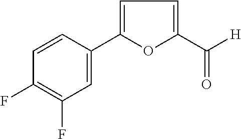 [SMILES: O=Cc1ccc(-c2ccc(F)c(F)c2)o1]

To a solution of 3,4-difluorophenyl boronic acid (1.0 g, 6.0 mmol) and 5-bromo-2-furaldehyde (1.1 g, 6.0 mmol) in 40 mL of a 4:1 mixture of toluene and ethanol at ambient temperature was added Palladium II acetate (67 mg, 0.3 mmol), triphenylphosphine (0.31 g, 1.2 mmol) and saturated aqueous sodium bicarbonate solution (6.7 mL). The mixture was heated at 80° C. for 22 h. After cooling to ambient temperature, the reaction mixture was partitioned between ethyl acetate and water. The phases were separated and the aqueous phase was extracted t with ethyl acetate. The combined organic phase was washed with water, then brine and dried over sodium sulfate. The solution was filtered, silica gel was added to the filtrate and the mixture was concentrated in vacuo. The residue was transferred to a pre-column and purified by chromatography using initially hexanes (4 min.) as eluent. The eluent was modified to 10% ethyl acetate/hexanes over a 10 min. period and held at 10% ethyl acetate/hexanes for the remainder of the purification. The fractions containing the pure desired product were combined and concentrated in vacuo to give 1.0 g (80.5% yield) of 5-(3,4-difluorophenyl)furan-2-carbaldehyde as an off-white solid.

MS: m/z 209 (MH⁺).

¹H NMR (500 MHz CDCl₃): δ 6.803 (d, 1H), 7.247 (m, 1H), 7.314 (d, 1H), 7.562 (m, 1H), 7.636 (m, 1H), 9.666 (s, 1H).

Step 2: 2-{(5Z)-5-{[5-(3,4-difluorophenyl)furan-2-yl]methylidene}-4-oxo-2-sulfanylidene-1,3-thiazolidin-3-yl}acetic acid: This compound was prepared in a similar fashion as described in Example 1 substituting 5-(3,4-difluorophenyl)furan-2-carbaldehyde for 5-(4-chlorophenyl)furan-2-carbaldehyde. The filtered solid was washed repeatedly with water, then diethyl ether to give a 80.3% yield of 2-{(5Z)-5-{[5-(3,4-difluorophenyl)furan-2-yl]methylidene}-4-oxo-2-sulfanylidene-1,3-thiazolidin-3-yl}acetic acid as a yellow solid.

MS: m/z 379.9 (MH⁻).

¹H NMR (500 MHz, DMSO-d₆): δ 4.562 (s, 2H), 7.387(d, 1H), 7.394 (d, 1H), 7.686 (m, 2H), 7.707 (s, 1H), 7.925 (d, 1H).

### Example H

### 2-[(5Z)-5-{[5-(3,4-Dichlorophenyl)furan-2-yl]methylidene}-4-oxo-2-sulfanylidene-1,3-thiazolidin-3-yl]ethane-1-sulfonic acid (BAS02250954)

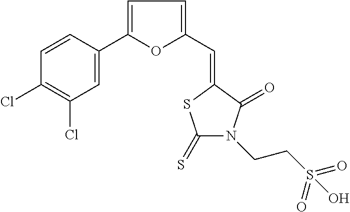 [SMILES: O=C1/C(=C/c2ccc(-c3ccc(Cl)c(Cl)c3)o2)SC(=S)N1CCS(=O)(=O)O]

### Example I

### 2-[(5Z)-5-{[5-(4-fluorophenyl)furan-2-yl]methylidene}-4-oxo-2-sulfanylidene-1,3-thiazolidin-3-yl]ethane-1-sulfonic acid (STK823366)

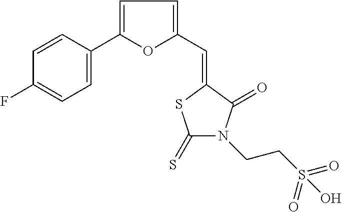 [SMILES: O=C1/C(=C/c2ccc(-c3ccc(F)cc3)o2)SC(=S)N1CCS(=O)(=O)O]

### Example J

### Propyl 2-chloro-5-(5-{[(5Z)-4-oxo-2-sulfanylidene-1,3-thiazolidin-5-ylidene]methyl}furan-2-yl)benzoate (BAS03338548)

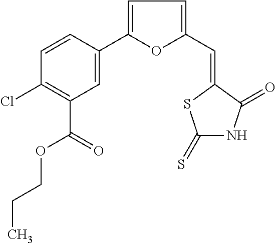 [SMILES: CCCOC(=O)c1cc(-c2ccc(/C=C3\SC(=S)NC3=O)o2)ccc1Cl]

### Example 6

### AMPK Activators for Treatment of Diabetes, Obesity and/or Metabolic Syndrome

Compounds will be tested in the C57-diet induced obesity (DIO) model for type-II diabetes, obesity and metabolic syndrome. Compounds will be administered at 10 mg/kg body weight via daily intraperitoneal injection. Endpoints for efficacy in diabetes, obesity and metabolic syndrome include, but are not limited to, improved glucose tolerance, decreased HbA1C Percentage below 6.5%, reduced body weight, decreased lipid accumulation in the liver and improved insulin resistance.

### Example 7

### AMPK Inhibitors for Treatment of Cancer

Compounds will be tested in a human tumor xenograft mouse model, where human tumor cells are transplanted into immunocompromised mice that do not reject human cells. The athymic nude mice will be used and several tumor types will be induced by transplantation including, but not limited to, breast cancer, colon cancer and pancreatic cancer. Compounds will be injected via intraperitoneal injection for 6-weeks. Endpoints for efficacy in this cancer model include, but are not limited to, reduced tumor burden/volume/load and reduced distant metastases.

1. An in vitro method for identifying a compound that modulates adenosine monophosphate-activated protein kinase (AMPK) for the manufacture of a diagnostic or therapeutic agent, comprising:
(a) contacting a sample comprising AMPK with a luminescent agent known to bind AMPK;
(b) contacting the sample from (a) with a compound of interest; and
(c) comparing the luminescence in the sample prior to contacting the sample with the compound of interest to the luminescence in the sample after contacting the sample with the compound of interest, wherein a decrease in luminescence after contacting the sample with the compound of interest indicates that the compound of interest is a modulator of AMPK.

2. The method of claim 1, wherein luminescence is detected using fluorimetry, fluorescence binding, fluorescence polarization, fluorescence resonance energy transfer (FRET) or time-resolved fluorescence resonance energy transfer (TR-FRET).

3-12. (canceled)

13. The method of claim 1, wherein the sample comprises a low concentration of AMPK and ADP or an analog thereof.

14. The method of claim 1 further comprising conducting a cell-based assay, an in vitro kinase assay, an in vitro phosphatase assay, or a combination thereof.

15-24. (canceled)

25. A method for identifying a compound that modulates adenosine monophosphate-activated protein kinase (AMPK), comprising:
(a) contacting a sample comprising AMPK with a luminescent agent known to bind AMPK;
(b) contacting the sample from (a) with a compound of interest; and
(c) comparing the luminescence in the sample prior to contacting the sample with the compound of interest to the luminescence in the sample after contacting the sample with the compound of interest, wherein:
(i) a decrease in luminescence after contacting the sample with the compound of interest indicates that the compound of interest is a modulator of AMPK, or
(ii) an increase in luminescence after contacting the sample with the compound of interest indicates that the compound of interest is a modulator of AMPK when the luminescent agent is environmentally sensitive.

26. A method of modulating the activity of adenosine monophosphate-activated protein kinase (AMPK), comprising contacting a sample comprising AMPK with a compound selected from the group consisting of:

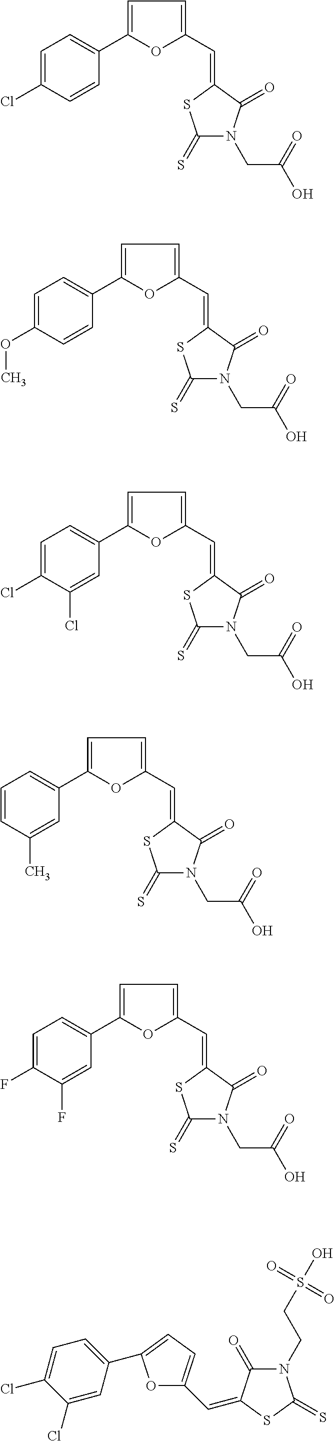 [SMILES: O=C(O)CN1C(=O)/C(=C/c2ccc(-c3ccc(Cl)cc3)o2)SC1=S ; COc1ccc(-c2ccc(/C=C3\SC(=S)N(CC(=O)O)C3=O)o2)cc1 ; O=C(O)CN1C(=O)/C(=C/c2ccc(-c3ccc(Cl)c(Cl)c3)o2)SC1=S ; Cc1cccc(-c2ccc(/C=C3\SC(=S)N(CC(=O)O)C3=O)o2)c1 ; O=C(O)CN1C(=O)/C(=C/c2ccc(-c3ccc(F)c(F)c3)o2)SC1=S ; O=C1/C(=C\c2ccc(-c3ccc(Cl)c(Cl)c3)o2)SC(=S)N1CCS(=O)(=O)O]

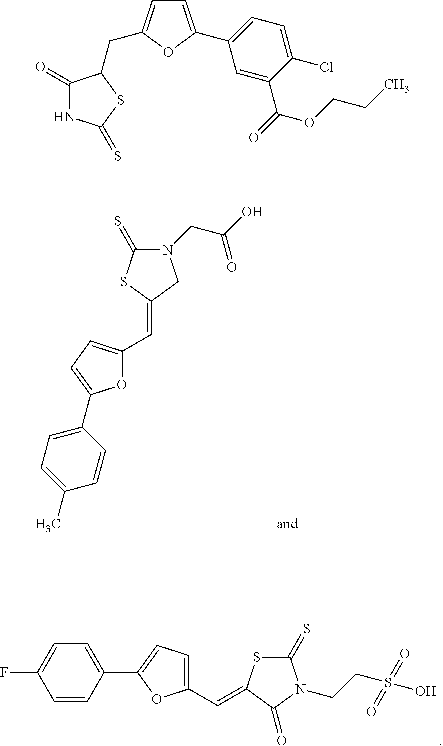 [SMILES: CCCOC(=O)c1cc(-c2ccc(CC3SC(=S)NC3=O)o2)ccc1Cl ; Cc1ccc(-c2ccc(/C=C3/CN(CC(=O)O)C(=S)S3)o2)cc1 ; O=C1/C(=C/c2ccc(-c3ccc(F)cc3)o2)SC(=S)N1CCS(=O)(=O)O]

27-32. (canceled)

33. A method of treating diabetes, obesity, metabolic syndrome or cancer comprising administering to a subject an effective amount of a compound selected from the group consisting of: 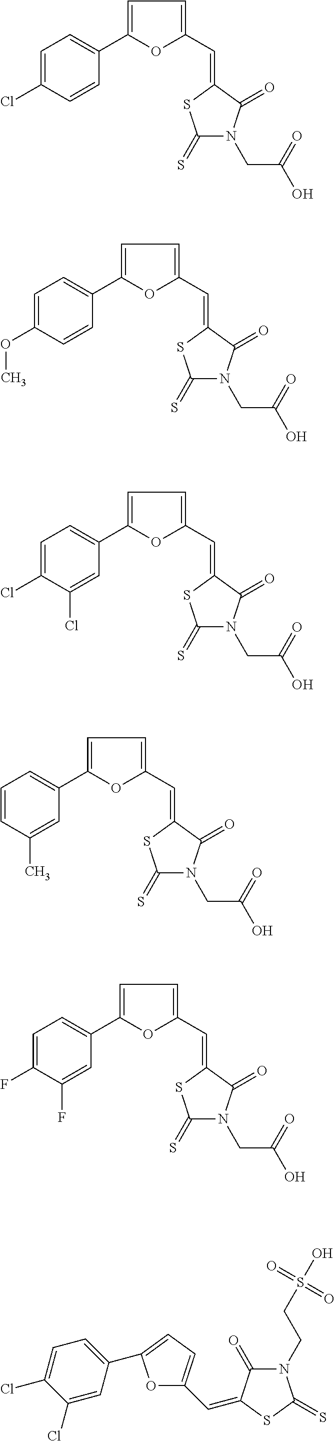 [SMILES: O=C(O)CN1C(=O)/C(=C/c2ccc(-c3ccc(Cl)cc3)o2)SC1=S ; COc1ccc(-c2ccc(/C=C3\SC(=S)N(CC(=O)O)C3=O)o2)cc1 ; O=C(O)CN1C(=O)/C(=C/c2ccc(-c3ccc(Cl)c(Cl)c3)o2)SC1=S ; Cc1cccc(-c2ccc(/C=C3\SC(=S)N(CC(=O)O)C3=O)o2)c1 ; O=C(O)CN1C(=O)/C(=C/c2ccc(-c3ccc(F)c(F)c3)o2)SC1=S ; O=C1/C(=C\c2ccc(-c3ccc(Cl)c(Cl)c3)o2)SC(=S)N1CCS(=O)(=O)O] 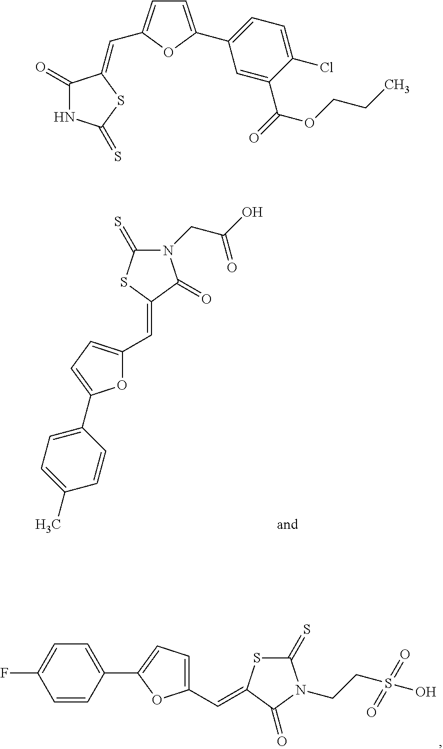 [SMILES: CCCOC(=O)c1cc(-c2ccc(/C=C3\SC(=S)NC3=O)o2)ccc1Cl ; Cc1ccc(-c2ccc(/C=C3\SC(=S)N(CC(=O)O)C3=O)o2)cc1 ; O=C1/C(=C/c2ccc(-c3ccc(F)cc3)o2)SC(=S)N1CCS(=O)(=O)O]
or a pharmaceutically acceptable salt thereof.

34. The method of claim 33, wherein the compound is selected from the group consisting of:

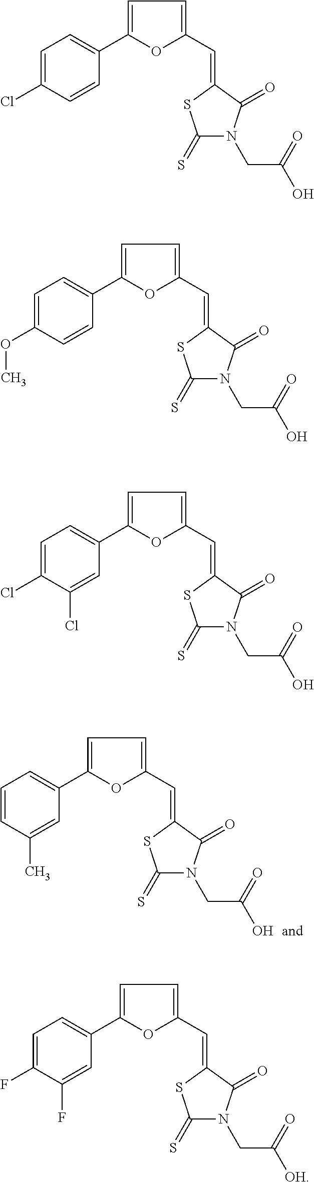 [SMILES: O=C(O)CN1C(=O)/C(=C/c2ccc(-c3ccc(Cl)cc3)o2)SC1=S ; COc1ccc(-c2ccc(/C=C3\SC(=S)N(CC(=O)O)C3=O)o2)cc1 ; O=C(O)CN1C(=O)/C(=C/c2ccc(-c3ccc(Cl)c(Cl)c3)o2)SC1=S ; Cc1cccc(-c2ccc(/C=C3\SC(=S)N(CC(=O)O)C3=O)o2)c1 ; O=C(O)CN1C(=O)/C(=C/c2ccc(-c3ccc(F)c(F)c3)o2)SC1=S]

35. A compound having the following structure: 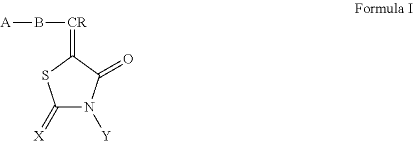
wherein:
A is a moiety selected from the group consisting of: aryl and benzyl; wherein each of said aryl or benzyl groups can optionally have from 0 to 3 substituents selected from the group consisting of: C₁-C₆ alkyl, C₃-C₇ cycloalkyl, benzyl, aryl, —(CO₂₋alkyl), alkoxy, aryloxy, amino, alkylamino, dialkylamino, amido, nitro and halogen;
B is a heterocyclic moiety selected from the group consisting of: substituted or unsubstituted furan, thiophene, azole, imidazole, pyrazole, oxazole, thiazole and isoxazole;
R is selected from the group consisting of: H, C₁-C₆ alkyl, C₃-C₇ cycloalkyl, benzyl and aryl wherein said benzyl and aryl substituents can optionally be unsubstituted or substituted with one or more groups selected from halogen; alkyl and alkoxy;
X═O or S;
Y is selected from the group consisting of: H, —(CH₂)ₙ—CO₂H, —(CH₂)ₙ—SO₃H, ArCO₂H and ArSO₃H, wherein each CH₂ group can optionally be substituted with one or two alkyl groups and each aryl group can optionally be substituted with one or more groups selected from: halogen; alkyl and alkoxy; and
n=1-6; wherein the stereochemistry of the double bond between the thiazolidenyl moiety and the carbon bearing R and B can be either E or Z; 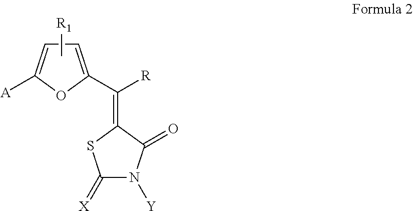
wherein A, R, X, and Y are defined as in Formula 1 and R₁ is selected from the group consisting of: H, C₁-C₆ alkyl, C₃-C₇ cycloalkyl, benzyl, aryl, alkoxy, aryloxy and halogen; 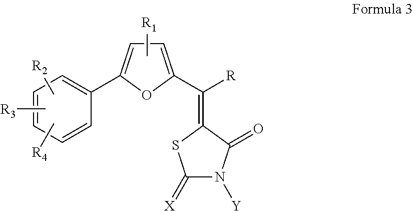
wherein R₁, R, X, and Y are defined as in Formula 2 and groups R₂₋₄ are independently selected from the group consisting of: H, C₁-C₆ alkyl, C₃-C₇ cycloalkyl, benzyl, aryl, —(CO₂₋alkyl), alkoxy, aryloxy and halogen; 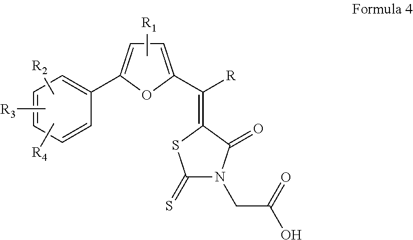
wherein R₁-R₄ are defined as in Formula 3; 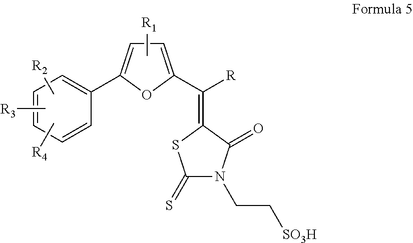
wherein R₁-R₄ are defined as in Formula 3; and 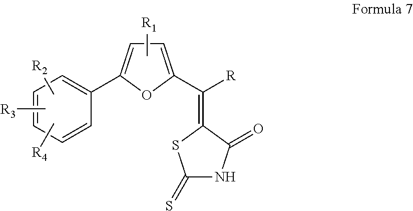
wherein R₁-R₄ are defined as in Formula 3, with the proviso that the compound is not

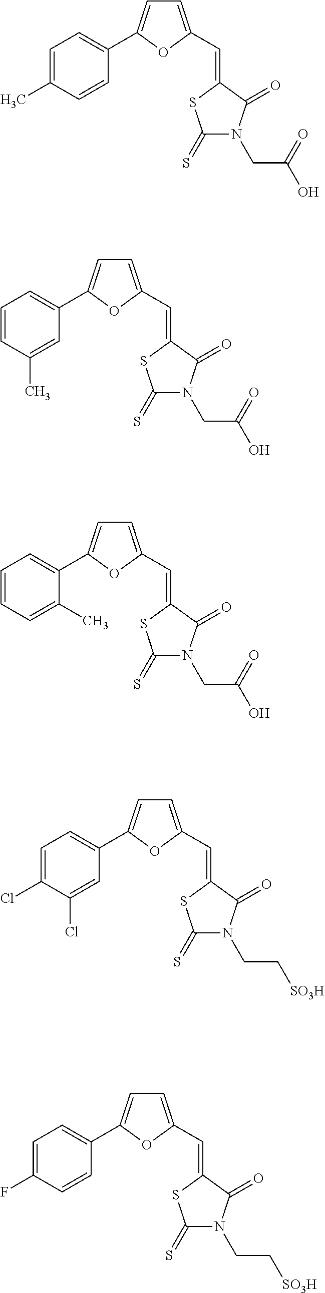 [SMILES: Cc1ccc(-c2ccc(/C=C3\SC(=S)N(CC(=O)O)C3=O)o2)cc1 ; Cc1cccc(-c2ccc(/C=C3\SC(=S)N(CC(=O)O)C3=O)o2)c1 ; Cc1ccccc1-c1ccc(/C=C2\SC(=S)N(CC(=O)O)C2=O)o1 ; O=C1/C(=C/c2ccc(-c3ccc(Cl)c(Cl)c3)o2)SC(=S)N1CCS(=O)(=O)O ; O=C1/C(=C/c2ccc(-c3ccc(F)cc3)o2)SC(=S)N1CCS(=O)(=O)O]

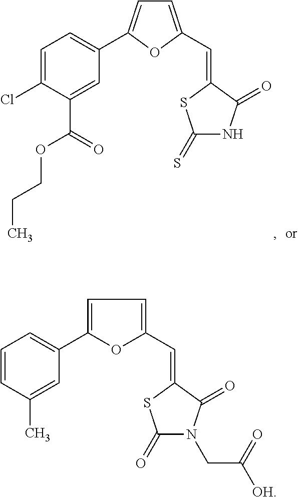 [SMILES: CCCOC(=O)c1cc(-c2ccc(/C=C3\SC(=S)NC3=O)o2)ccc1Cl ; Cc1cccc(-c2ccc(/C=C3\SC(=O)N(CC(=O)O)C3=O)o2)c1]

36. A compound having the following structure and pharmaceutically acceptable salts thereof, or a pharmaceutical composition of the same comprising a pharmaceutically acceptable carrier: 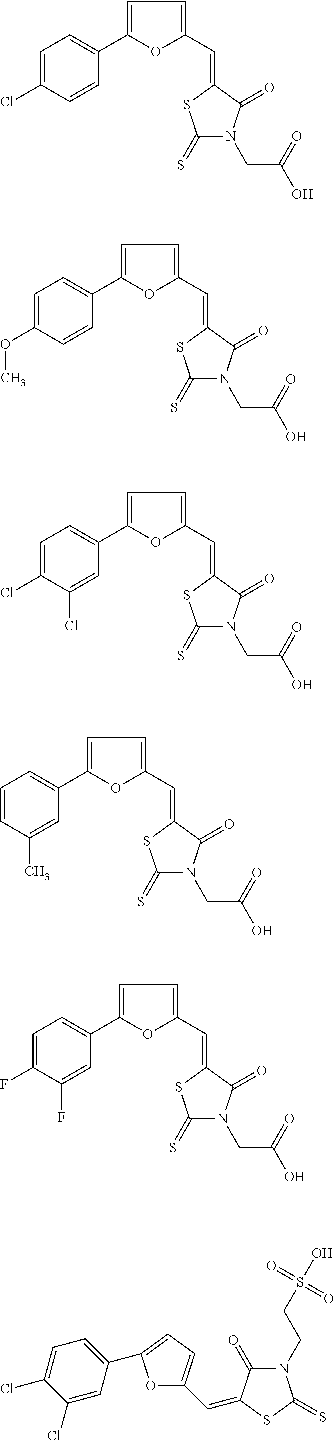 [SMILES: O=C(O)CN1C(=O)/C(=C/c2ccc(-c3ccc(Cl)cc3)o2)SC1=S ; COc1ccc(-c2ccc(/C=C3\SC(=S)N(CC(=O)O)C3=O)o2)cc1 ; O=C(O)CN1C(=O)/C(=C/c2ccc(-c3ccc(Cl)c(Cl)c3)o2)SC1=S ; Cc1cccc(-c2ccc(/C=C3\SC(=S)N(CC(=O)O)C3=O)o2)c1 ; O=C(O)CN1C(=O)/C(=C/c2ccc(-c3ccc(F)c(F)c3)o2)SC1=S ; O=C1/C(=C\c2ccc(-c3ccc(Cl)c(Cl)c3)o2)SC(=S)N1CCS(=O)(=O)O] 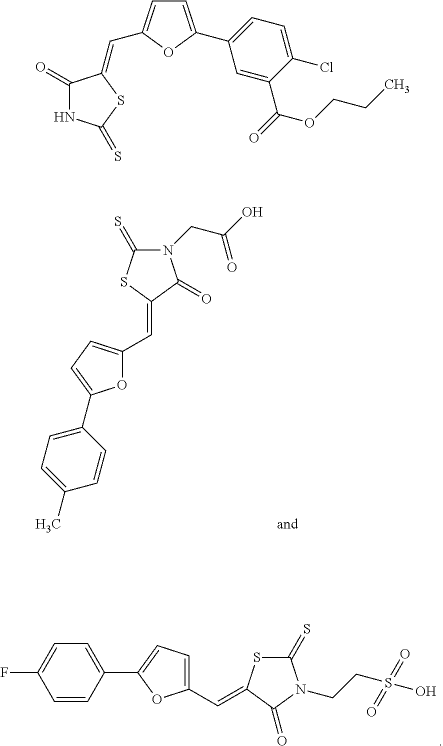 [SMILES: CCCOC(=O)c1cc(-c2ccc(/C=C3\SC(=S)NC3=O)o2)ccc1Cl ; Cc1ccc(-c2ccc(/C=C3\SC(=S)N(CC(=O)O)C3=O)o2)cc1 ; O=C1/C(=C/c2ccc(-c3ccc(F)cc3)o2)SC(=S)N1CCS(=O)(=O)O]


SEQUENCE LISTING (25 sequences)

SEQ ID NO: 1 - protein, 330 aa, Rattus norvegicus
MESVAAESAPAPENEHSQETPESNSSVYTTFMKSHRCYDLIPTSSKLVVFDTSLQVKKAFFALVTNGVRAAPLWDSKKQSFVGMLTITDFINILHRYYKSALVQIYELEEHKIETWREVYLQDSFKPLVCISPNASLFDAVSSLIRNKIHRLPVIDPESGNTLYILTHKRILKFLKLFITEFPKPEFMSKSLEELQIGTYANIAMVRTTTPVYVALGIFVQHRVSALPVVDEKGRVVDIYSKFDVINLAAEKTYNNLDVSVTKALQHRSHYFEGVLKCYLHETLEAIINRLVEAEVHRLVVVDEHDVVKGIVSLSDILQALVLTGGEKKP

SEQ ID NO: 2 - protein, 270 aa, Rattus norvegicus
MGNTSSERAALERQAGHKTPRRDSSGGTKDGDRPKILMDSPEDADIFHTEEMKAPEKEEFLAWQHDLEVNEKAPAQARPTVFRWTGGGKEVYLSGSFNNWSKLPLTRSQNNFVAILDLPEGEHQYKFFVDGQWTHDPSEPIVTSQLGTVNNIIQVKKTDFEVFDALMVDSQKCSDVSELSSSPPGPYHQEPYISKPEERFKAPPILPPHLLQVILNKDTGISCDPALLPEPNHVMLNHLYALSIKDGVMVLSATHRYKKKYVTTLLYKPI

SEQ ID NO: 3 - protein, 548 aa, Rattus norvegicus
MAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRATIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTFSKMSLQLYQVDSRTYLLDFRSIDDEITEAKSGTATPQRSGSISNYRSCQRSDSDAEAQGKPSEVSLTSSVTSLDSSPVDVAPRPGSHTIEFFEMCANLIKILAQ

SEQ ID NO: 4 - protein, 153 aa, Rattus norvegicus
WHLGIRSQSRPNDIMAEVCRAIKQLDYEWKVVNPYYLRVRRKNPVTSTFSKMSLQLYQVDSRTYLLDFRSIDDEITEAKSGTATPQRSGSISNYRSCQRSDSDAEAQGKPSEVSLTSSVTSLDSSPVDVAPRPGSHTIEFFEMCANLIKILAQ

SEQ ID NO: 5 - protein, 269 aa, Rattus norvegicus
VKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRATIKDIREHEWFKQDLPKYLFPE

SEQ ID NO: 6 - protein, 552 aa, Rattus norvegicus
MAEKQKHDGRVKIGHYVLGDTLGVGTFGKVKIGEHQLTGHKVAVKILNRQKIRSLDVVGKIKREIQNLKLFRHPHIIKLYQVISTPTDFFMVMEYVSGGELFDYICKHGRVEEVEARRLFQQILSAVDYCHRHMVVHRDLKPENVLLDAQMNAKIADFGLSNMMSDGEFLRTSCGSPNYAAPEVISGRLYAGPEVDIWSCGVILYALLCGTLPFDDEHVPTLFKKIRGGVFYIPEYLNRSIATLLMHMLQVDPLKRATIKDIREHEWFKQDLPSYLFPEDPSYDANVIDDEAVKEVCEKFECTESEVMNSLYSGDPQDQLAVAYHLIIDNRRIMNQASEFYLASSPPTGSFMDDSAMHIPPGLKPHPERMPPLIADSPKARCPLDALNTTKPKSLAVKKAKWHLGIRSQSKPYDIMAEVYRAMKQLDFEWKVVNAYHLRVRRKNPVTGNYVKMSLQLYLVDNRSYLLDFKSIDDEVVEQRSGSSTPQRSCSAAGLHRPRSSVDSSTAENHSLSGSLTGSLTGSTLSSASPRLGSHTMDFFEMCASLITALAR

SEQ ID NO: 7 - protein, 268 aa, Rattus norvegicus
VKIGHYVLGDTLGVGTFGKVKIGEHQLTGHKVAVKILNRQKIRSLDVVGKIKREIQNLKLFRHPHIIKLYQVISTPTDFFMVMEYVSGGELFDYICKHGRVEEVEARRLFQQILSAVDYCHRHMVVHRDLKPENVLLDAQMNAKIADFGLSNMMSDGEFLRTSCGSPNYAAPEVISGRLYAGPEVDIWSCGVILYALLCGTLPFDDEHVPTLFKKIRGGVFYIPEYLNRSIATLLMHMLQVDPLKRATIKDIREHEWFKQDLPSYLFP

SEQ ID NO: 8 - protein, 151 aa, Rattus norvegicus
WHLGIRSQSKPYDIMAEVYRAMKQLDFEWKVVNAYHLRVRRKNPVTGNYVKMSLQLYLVDNRSYLLDFKSIDDEVVEQRSGSSTPQRSCSAAGLHRPRSSVDSSTAENHSLSGSLTGSLTGSTLSSASPRLGSHTMDFFEMCASLITALAR

SEQ ID NO: 9 - protein, 271 aa, Rattus norvegicus
MGNTTSERVSGERHGAKAARAEGGGHGPGKEHKIMVGSTDDPSVFSLPDSKLPGDKEFVPWQQDLDDSVKPTQQARPTVIRWSEGGKEVFISGSFNNWSTKIPLIKSHNDFVAILDLPEGEHQYKFFVDGQWVHDPSEPVVTSQLGTINNLIHVKKSDFEVFDALKLDSMESSETSCRDLSSSPPGPYGQEMYVFRSEERFKSPPILPPHLLQVILNKDTNISCDPALLPEPNHVMLNHLYALSTKDSVMVLSATHRYKKKYVTTLLYKPI

SEQ ID NO: 10 - protein, 326 aa, Rattus norvegicus
MLEKLEFQEEEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPNTPIIKALNIFVERRISALPVVDESGKVVDIYSKFDVINLAAEKTYNNLDITVTQALQHRSQYFEGVVKCSKLETLETIVDRIVRAEVHRLVVVNEADSIVGIISLSDILQALILTPAGAKQKETETE

SEQ ID NO: 11 - protein, 331 aa, Homo sapiens
METVISSDSSPAVENEHPQETPESNNSVYTSFMKSHRCYDLIPTSSKLVVFDTSLQVKKAFFALVTNGVRAAPLWDSKKQSFVGMLTITDFINILHRYYKSALVQIYELEEHKIETWREVYLQDSFKPLVCISPNASLFDAVSSLIRNKIHRLPVIDPESGNTLYILTHKRILKFLKLFITEFPKPEFMSKSLEELQIGTYANIAMVRTTTPVYVALGIFVQHRVSALPVVDEKGRVVDIYSKFDVINLAAEKTYNNLDVSVTKALQHRSHYFEGVLKCYLHETLETIINRLVEAEVHRLVVVDENDVVKGIVSLSDILQALVLTGGEKKP

SEQ ID NO: 12 - protein, 569 aa, Homo sapiens
MGSAVMDTKKKKDVSSPGGSGGKKNASQKRRSLRVHIPDLSSFAMPLLDGDLEGSGKHSSRKVDSPFGPGSPSKGFFSRGPQPRPSSPMSAPVRPKTSPGSPKTVFPFSYQESPPRSPRRMSFSGIFRSSSKESSPNSNPATSPGGIRFFSRSRKTSGLSSSPSTPTQVTKQHTFPLESYKHEPERLENRIYASSSPPDTGQRFCPSSFQSPTRPPLASPTHYAPSKAAALAAALGPAEAGMLEKLEFEDEAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 13 - protein, 328 aa, Homo sapiens
MLEKLEFEDEAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 14 - protein, 525 aa, Homo sapiens
MPLLDGDLEGSGKHSSRKVDSPFGPGSPSKGFFSRGPQPRPSSPMSAPVRPKTSPGSPKTVFPFSYQESPPRSPRRMSFSGIFRSSSKESSPNSNPATSPGGIRFFSRSRKTSGLSSSPSTPTQVTKQHTFPLESYKHEPERLENRIYASSSPPDTGQRFCPSSFQSPTRPPLASPTHYAPSKAAALAAALGPAEAGMLEKLEFEDEAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 15 - protein, 489 aa, Homo sapiens
MEPGLEHALRRTPSWSSLGGSEHQEMSFLEQENSSSWPSPAVTSSSERIRGKRRAKALRWTRQKSVEEGEPPGQGEGPRSRPAAESTGLEATFPKTTPLAQADPAGVGTPPTGWDCLPSDCTASAAGSSTDDVELATEFPATEAWECELEGLLEERPALCLSPQAPFPKLGWDDELRKPGAQIYMRFMQEHTCYDAMATSSKLVIFDTMLEIKKAFFALVANGVRAAPLWDSKKQSFVGMLTITDFILVLHRYYRSPLVQIYEIEQHKIETWREIYLQGCFKPLVSISPNDSLFEAVYTLIKNRIHRLPVLDPVSGNVLHILTHKRLLKFLHIFGSLLPRPSFLYRTIQDLGIGTFRDLAVVLETAPILTALDIFVDRRVSALPVVNECGQVVGLYSRFDVIHLAAQQTYNHLDMSVGEALRQRTLCLEGVLSCQPHESLGEVIDRIAREQVHRLVLVDETQHLLGVVSLSDILQALVLSPAGIDALGA

SEQ ID NO: 16 - protein, 270 aa, Homo sapiens
MGNTSSERAALERHGGHKTPRRDSSGGTKDGDRPKILMDSPEDADLFHSEEIKAPEKEEFLAWQHDLEVNDKAPAQARPTVFRWTGGGKEVYLSGSFNNWSKLPLTRSHNNFVAILDLPEGEHQYKFFVDGQWTHDPSEPIVTSQLGTVNNIIQVKKTDFEVFDALMVDSQKCSDVSELSSSPPGPYHQEPYVCKPEERFRAPPILPPHLLQVILNKDTGISCDPALLPEPNHVMLNHLYALSIKDGVMVLSATHRYKKKYVTTLLYKPI

SEQ ID NO: 17 - protein, 272 aa, Homo sapiens
MGNTTSDRVSGERHGAKAARSEGAGGHAPGKEHKIMVGSTDDPSVFSLPDSKLPGDKEFVSWQQDLEDSVKPTQQARPTVIRWSEGGKEVFISGSFNNWSTKIPLIKSHNDFVAILDLPEGEHQYKFFVDGQWVHDPSEPVVTSQLGTINNLIHVKKSDFEVFDALKLDSMESSETSCRDLSSSPPGPYGQEMYAFRSEERFKSPPILPPHLLQVILNKDTNISCDPALLPEPNHVMLNHLYALSIKDSVMVLSATHRYKKKYVTTLLYKPI

SEQ ID NO: 18 - protein, 86 aa, Homo sapiens
GPYGQEMYAFRSEERFKSPPILPPHLLQVILNKDTNISCDPALLPEPNHVMLNHLYALSIKDSVMVLSATHRYKKKYVTTLLYKPI

SEQ ID NO: 19 - protein, 559 aa, Homo sapiens
MRRLSSWRKMATAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRATIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 20 - protein, 268 aa, Homo sapiens
VKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRATIKDIREHEWFKQDLPKYLFP

SEQ ID NO: 21 - protein, 153 aa, Homo sapiens
WHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 22 - protein, 552 aa, Homo sapiens
MAEKQKHDGRVKIGHYVLGDTLGVGTFGKVKIGEHQLTGHKVAVKILNRQKIRSLDVVGKIKREIQNLKLFRHPHIIKLYQVISTPTDFFMVMEYVSGGELFDYICKHGRVEEMEARRLFQQILSAVDYCHRHMVVHRDLKPENVLLDAHMNAKIADFGLSNMMSDGEFLRTSCGSPNYAAPEVISGRLYAGPEVDIWSCGVILYALLCGTLPFDDEHVPTLFKKIRGGVFYIPEYLNRSVATLLMHMLQVDPLKRATIKDIREHEWFKQDLPSYLFPEDPSYDANVIDDEAVKEVCEKFECTESEVMNSLYSGDPQDQLAVAYHLIIDNRRIMNQASEFYLASSPPSGSFMDDSAMHIPPGLKPHPERMPPLIADSPKARCPLDALNTTKPKSLAVKKAKWHLGIRSQSKPYDIMAEVYRAMKQLDFEWKVVNAYHLRVRRKNPVTGNYVKMSLQLYLVDNRSYLLDFKSIDDEVVEQRSGSSTPQRSCSAAGLHRPRSSFDSTTAESHSLSGSLTGSLTGSTLSSVSPRLGSHTMDFFEMCASLITTLAR

SEQ ID NO: 23 - protein, 268 aa, Homo sapiens
VKIGHYVLGDTLGVGTFGKVKIGEHQLTGHKVAVKILNRQKIRSLDVVGKIKREIQNLKLFRHPHIIKLYQVISTPTDFFMVMEYVSGGELFDYICKHGRVEEMEARRLFQQILSAVDYCHRHMVVHRDLKPENVLLDAHMNAKIADFGLSNMMSDGEFLRTSCGSPNYAAPEVISGRLYAGPEVDIWSCGVILYALLCGTLPFDDEHVPTLFKKIRGGVFYIPEYLNRSVATLLMHMLQVDPLKRATIKDIREHEWFKQDLPSYLFP

SEQ ID NO: 24 - protein, 151 aa, Homo sapiens
WHLGIRSQSKPYDIMAEVYRAMKQLDFEWKVVNAYHLRVRRKNPVTGNYVKMSLQLYLVDNRSYLLDFKSIDDEVVEQRSGSSTPQRSCSAAGLHRPRSSFDSTTAESHSLSGSLTGSLTGSTLSSVSPRLGSHTMDFFEMCASLITTLAR

SEQ ID NO: 25 - protein, 28 aa, Artificial
  GST-ACC peptide sequence
GGGGGLAFHMRSSMSGLHLVKQGRDRKK